SEQUENCE LISTING (101 sequences)

SEQ ID NO: 1 - DNA, 1596 bp, Homo sapiens
atggctcccagcagcccccggcccgcgctgcccgcactcctggtcctgctcggggctctgttcccaggacctggcaatgcccagacatctgtgtccccctcaaaagtcatcctgccccggggaggctccgtgctggtgacatgcagcacctcctgtgaccagcccaagttgttgggcatagagaccccgttgcctaaaaaggagttgctcctgcctgggaacaaccggaaggtgtatgaactgagcaatgtgcaagaagatagccaaccaatgtgctattcaaactgccctgatgggcagtcaacagctaaaaccttcctcaccgtgtactggactccagaacgggtggaactggcacccctcccctcttggcagccagtgggcaagaaccttaccctacgctgccaggtggagggtggggcaccccgggccaacctcaccgtggtgctgctccgtggggagaaggagctgaaacgggagccagctgtgggggagcccgctgaggtcacgaccacggtgctggtgaggagagatcaccatggagccaatttctcgtgccgcactgaactggacctgcggccccaagggctggagctgtttgagaacacctcggccccctaccagctccagacctttgtcctgccagcgactcccccacaacttgtcagcccccgggtcctagaggtggacacgcaggggaccgtggtctgttccctggacgggctgttcccagtctcggaggcccaggtccacctggcactgggggaccagaggttgaaccccacagtcacctatggcaacgactccttctcggccaaggcctcagtcagtgtgaccgcagaggacgagggcacccagcggctgacgtgtgcagtaatactggggaaccagagccaggagacactgcagacagtgaccatctacagctttccggcgcccaacgtgattctgacgaagccagaggtctcagaagggaccgaggtgacagtgaagtgtgaggcccaccctagagccaaggtgacgctgaatggggttccagcccagccactgggcccgagggcccagctcctgctgaaggccaccccagaggacaacgggcgcagcttctcctgctctgcaaccctggaggtggccggccagcttatacacaagaaccagacccgggagcttcgtgtcctgtatggcccccgactggacgagagggattgtccgggaaactggacgtggccagaaaattcccagcagactccaatgtgccaggcttgggggaacccattgcccgagctcaagtgtctaaaggatggcactttcccactgcccatcggggaatcagtgactgtcactcgagatcttgagggcacctacctctgtcgggccaggagcactcaaggggaggtcacccgcaaggtgaccgtgaatgtgctctccccccggtatgagattgtcatcatcactgtggtagcagccgcagtcataatgggcactgcaggcctcagcacgtacctctataaccgccagcggaagatcaagaaatacagactacaacaggcccaaaaagggacccccatgaaaccgaacacacaagccacgcctccc

SEQ ID NO: 2 - protein, 532 aa, Homo sapiens
MAPSSPRPALPALLVLLGALFPGPGNAQTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTRKVTVNVLSPRYEIVIITVVAAAVIMGTAGLSTYLYNRQRKIKKYRLQQAQKGTPMKPNTQATPP

SEQ ID NO: 3 - DNA, 3003 bp, Homo sapiens
gctataaggatcacgcgccccagtcgacgctgagctcctctgctactcagagttgcaacctcagcctcgctatggctcccagcagcccccggcccgcgctgcccgcactcctggtcctgctcggggctctgttcccaggacctggcaatgcccagacatctgtgtccccctcaaaagtcatcctgccccggggaggctccgtgctggtgacatgcagcacctcctgtgaccagcccaagttgttgggcatagagaccccgttgcctaaaaaggagttgctcctgcctgggaacaaccggaaggtgtatgaactgagcaatgtgcaagaagatagccaaccaatgtgctattcaaactgccctgatgggcagtcaacagctaaaaccttcctcaccgtgtactggactccagaacgggtggaactggcacccctcccctcttggcagccagtgggcaagaaccttaccctacgctgccaggtggagggtggggcaccccgggccaacctcaccgtggtgctgctccgtggggagaaggagctgaaacgggagccagctgtgggggagcccgctgaggtcacgaccacggtgctggtgaggagagatcaccatggagccaatttctcgtgccgcactgaactggacctgcggccccaagggctggagctgtttgagaacacctcggccccctaccagctccagacctttgtcctgccagcgactcccccacaacttgtcagcccccgggtcctagaggtggacacgcaggggaccgtggtctgttccctggacgggctgttcccagtctcggaggcccaggtccacctggcactgggggaccagaggttgaaccccacagtcacctatggcaacgactccttctcggccaaggcctcagtcagtgtgaccgcagaggacgagggcacccagcggctgacgtgtgcagtaatactggggaaccagagccaggagacactgcagacagtgaccatctacagctttccggcgcccaacgtgattctgacgaagccagaggtctcagaagggaccgaggtgacagtgaagtgtgaggcccaccctagagccaaggtgacgctgaatggggttccagcccagccactgggcccgagggcccagctcctgctgaaggccaccccagaggacaacgggcgcagcttctcctgctctgcaaccctggaggtggccggccagcttatacacaagaaccagacccgggagcttcgtgtcctgtatggcccccgactggacgagagggattgtccgggaaactggacgtggccagaaaattcccagcagactccaatgtgccaggcttgggggaacccattgcccgagctcaagtgtctaaaggatggcactttcccactgcccatcggggaatcagtgactgtcactcgagatcttgagggcacctacctctgtcgggccaggagcactcaaggggaggtcacccgcaaggtgaccgtgaatgtgctctccccccggtatgagattgtcatcatcactgtggtagcagccgcagtcataatgggcactgcaggcctcagcacgtacctctataaccgccagcggaagatcaagaaatacagactacaacaggcccaaaaagggacccccatgaaaccgaacacacaagccacgcctccctgaacctatcccgggacagggcctcttcctcggccttcccatattggtggcagtggtgccacactgaacagagtggaagacatatgccatgcagctacacctaccggccctgggacgccggaggacagggcattgtcctcagtcagatacaacagcatttggggccatggtacctgcacacctaaaacactaggccacgcatctgatctgtagtcacatgactaagccaagaggaaggagcaagactcaagacatgattgatggatgttaaagtctagcctgatgagaggggaagtggtgggggagacatagccccaccatgaggacatacaactgggaaatactgaaacttgctgcctattgggtatgctgaggccccacagacttacagaagaagtggccctccatagacatgtgtagcatcaaaacacaaaggcccacacttcctgacggatgccagcttgggcactgctgtctactgaccccaacccttgatgatatgtatttattcatttgttattttaccagctatttattgagtgtcttttatgtaggctaaatgaacataggtctctggcctcacggagctcccagtccatgtcacattcaaggtcaccaggtacagttgtacaggttgtacactgcaggagagtgcctggcaaaaagatcaaatggggctgggacttctcattggccaacctgcctttccccagaaggagtgatttttctatcggcacaaaagcactatatggactggtaatggttcacaggttcagagattacccagtgaggccttattcctcccttccccccaaaactgacacctttgttagccacctccccacccacatacatttctgccagtgttcacaatgacactcagcggtcatgtctggacatgagtgcccagggaatatgcccaagctatgccttgtcctcttgtcctgtttgcatttcactgggagcttgcactattgcagctccagtttcctgcagtgatcagggtcctgcaagcagtggggaagggggccaaggtattggaggactccctcccagctttggaagcctcatccgcgtgtgtgtgtgtgtgtgtatgtgtagacaagctctcgctctgtcacccaggctggagtgcagtggtgcaatcatggttcactgcagtcttgaccttttgggctcaagtgatcctcccacctcagcctcctgagtagctgggaccataggctcacaacaccacacctggcaaatttgattttttttttttttttcagagacggggtctcgcaacattgcccagacttcctttgtgttagttaataaagctttctcaactgcc

SEQ ID NO: 4 - protein, 6 aa, Homo sapiens
SEKDEL

SEQ ID NO: 5 - protein, 7 aa, Homo sapiens
RSEKDEL

SEQ ID NO: 6 - DNA, 52 bp, Artificial Sequence
  Description of Artificial Sequence: Cloning primer
tctgttcccaggaactagtttggcacagacatctgtgtccccctcaaaagtc

SEQ ID NO: 7 - DNA, 38 bp, Artificial Sequence
  Description of Artificial Sequence: Cloning primer
cataccggggactagtcacattcacggtcacctcgcgg

SEQ ID NO: 8 - protein, 799 aa, Homo sapiens
QTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTREVTVNVTSGSSASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSRVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRLAGKPTHINVSVVMAEADGTCYRSEKDEL

SEQ ID NO: 9 - DNA, 6313 bp, Artificial Sequence
  Description of Artificial Sequence: Expression-type plasmid pSSPICAMHuA2
gaactcgagcagctgaagcttgcatgcctgcaggtcgacggtatcgataaggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccgcgagctggtcaatcccattgcttttgaagcagctcaacattgatctctttctcgagggagatttttcaaatcagtgcgcaagacgtgacgtaagtatccgagtcagtttttatttttctactaatttggtcgtttatttcggcgtgtaggacatggcaaccgggcctgaatttcgcgggtattctgtttctattccaactttttcttgatccgcagccattaacgacttttgaatagatacgctgacacgccaagcctcgctagtcaaaagtgtaccaaacaacgctttacagcaagaacggaatgcgcgtgacgctcgcggtgacgccatttcgccttttcagaaatggataaatagccttgcttcctattatatcttcccaaattaccaatacattacactagcatctgaatttcataaccaatctcgatacaccaaatcgactctagaggatctatcgattcccgggtaccatgggatctaaaccttttttgtctcttctttcattgtcattgcttttgtttacatctactagtttggcacagacatctgtgtccccctcaaaagtcatcctgccccggggaggctccgtgctggtgacatgcagcacctcctgtgaccagcccaagttgttgggcatagagaccccgttgcctaaaaaggagttgctcctgcctgggaacaaccggaaggtgtatgaactgagcaatgtgcaagaagatagccaaccaatgtgctattcaaactgccctgatgggcagtcaacagctaaaaccttcctcaccgtgtactggactccagaacgggtggaactggcacccctcccctcttggcagccagtgggcaagaaccttaccctacgctgccaggtggagggtggggcaccccgggccaacctcaccgtggtgctgctccgtggggagaaggagctgaaacgggagccagctgtgggggagcccgctgaggtcacgaccacggtgctggtgaggagagatcaccatggagccaatttctcgtgccgcactgaactggacctgcggccccaagggctggagctgtttgagaacacctcggccccctaccagctccagacctttgtcctgccagcgactcccccacaacttgtcagcccccgggtcctagaggtggacacgcaggggaccgtggtctgttccctggacgggctgttcccagtctcggaggcccaggtccacctggcactgggggaccagaggttgaaccccacagtcacctatggcaacgactccttctcggccaaggcctcagtcagtgtgaccgcagaggacgagggcacccagcggctgacgtgtgcagtaatactggggaaccagagccaggagacactgcagacagtgaccatctacagctttccggcgcccaacgtgattctgacgaagccagaggtctcagaagggaccgaggtgacagtgaagtgtgaggcccaccctagagccaaggtgacgctgaatggggttccagcccagccactgggcccgagggcccagctcctgctgaaggccaccccagaggacaacgggcgcagcttctcctgctctgcaaccctggaggtggccggccagcttatacacaagaaccagacccgggagcttcgtgtcctgtatggcccccgactggacgagagggattgtccgggaaactggacgtggccagaaaattcccagcagactccaatgtgccaggcttgggggaacccattgcccgagctcaagtgtctaaaggatggcactttcccactgcccatcggggaatcagtgactgtcactcgagatcttgagggcacctacctctgtcgggccaggagcactcaaggggaggtcacccgcgaggtgaccgtgaatgtgactagtgggagctcagcatccccgaccagccccaaggtcttcccgctgagcctcgacagcaccccccaagatgggaacgtggtcgtcgcatgcctggtccagggcttcttcccccaggagccactcagtgtgacctggagcgaaagcggacagaacgtgaccgccagaaacttcccacctagccaggatgcctccggggacctgtacaccacgagcagccagctgaccctgccggccacacagtgcccagacggcaagtccgtgacatgccacgtgaagcactacacgaattccagccaggatgtgactgtgccctgccgagttcccccacctcccccatgctgccacccccgactgtcgctgcaccgaccggccctcgaggacctgctcttaggttcagaagcgaacctcacgtgcacactgaccggcctgagagatgcctctggtgccaccttcacctggacgccctcaagtgggaagagcgctgttcaaggaccacctgagcgtgacctctgtggctgctacagcgtgtccagagtacttcctggctgtgcccagccatggaaccatggggagaccttcacctgcactgctgcccaccccgagttgaagaccccactaaccgccaacatcacaaaatccggaaacacattccggcccgaggtccacctgctgccgccgccgtcggaggagctggccctgaacgagctggtgacgctgacgtgcctggcacgtggcttcagccccaaggatgtgctggttcgctggctgcaggggtcacaggagctgccccgcgagaagtacctgacttgggcatcccggcaggagcccagccagggcaccaccacctatgctgtgaccagcatactgcgcgtggcagccgaggactggaagaagggggagaccttctcctgcatggtgggccacgaggccctgccgctggccttcacacagaagaccatcgaccgcttggcgggtaaacccacccatatcaatgtgtctgttgtcatggcggaggcggacggcacctgctacagatctgaaaaggatgaactttagaattcgatatcaagctaattcccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggggatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactata

SEQ ID NO: 10 - protein, 22 aa, Phaseolus vulgaris
MSKPFLSLLSLSLLLFTSTCLA

SEQ ID NO: 11 - DNA, 508 bp, Artificial Sequence
  Description of Artificial Sequence: Protein coding region of the plasmid pSHuJ
aggatctatcgattcccgggtaccatggagaaccatttgcttttctggggagtcctggcggtttttattaaggctgttcatgtgaaagcccaagaagatgaaaggattgttcttgttgacaacaaatgtaagtgtgcccggattacttccaggatcatccgttcttccgaagatcctaatgaggacattgtggagagaaacatccgaattattgttcctctgaacaacagggagaatatctctgatcccacctcaccattgagaaccagatttgtgtaccatttgtctgacctctgtaaaaaatgtgatcctacagaagtggagctggataatcagatagttactgctacccagagcaatatctgtgatgaagacagtgctacagagacctgctacacttatgacagaaacaagtgctacacagctgtggtcccactcgtatatggtggtgagaccaaaatggtggaaacagccttaaccccagatgcctgctatcctgactgaattc

SEQ ID NO: 12 - DNA, 1845 bp, Artificial Sequence
  Description of Artificial Sequence: Protein coding region of plasmid pSHuSC
gtcgattcccgggtaccatggtgctcttcgtgctcacctgcctgctggcggtcttcccagccatctccacgaagagtcccatatttggtcccgaggaggtgaatagtgtggaaggtaactcagtgtccatcacgtgctactacccacccacctctgtcaaccggcacacccggaagtactggtgccggcagggagctagaggtggctgcataaccctcatctcctcggagggctacgtctccagcaaatatgcaggcagggctaacctcaccaacttcccggagaacggcacatttgtggtgaacattgcccagctgagccaggatgactccgggcgctacaagtgtggcctgggcatcaatagccgaggcctgtcctttgatgtcagcctggaggtcagccagggtcctgggctcctaaatgacactaaagtctacacagtggacctgggcagaacggtgaccatcaactgccctttcaagactgagaatgctcaaaagaggaagtccttgtacaagcagataggcctgtaccctgtgctggtcatcgactccagtggttatgtgaatcccaactatacaggaagaatacgccttgatattcagggtactggccagttactgttcagcgttgtcatcaaccaactcaggctcagcgatgctgggcagtatctctgccaggctggggatgattccaatagtaataagaagaatgctgacctccaagtgctaaagcccgagcccgagctggtttatgaagacctgaggggctcagtgaccttccactgtgccctgggccctgaggtggcaaacgtggccaaatttctgtgccgacagagcagtggggaaaactgtgacgtggtcgtcaacaccctggggaagagggccccagcctttgagggcaggatcctgctcaacccccaggacaaggatggctcattcagtgtggtgatcacaggcctgaggaaggaggatgcagggcgctacctgtgtggagcccattcggatggtcagctgcaggaaggctcgcctatccaggcctggcaactcttcgtcaatgaggagtccacgattccccgcagccccactgtggtgaagggggtggcaggaagctctgtggccgtgctctgcccctacaaccgtaaggaaagcaaaagcatcaagtactggtgtctctgggaaggggcccagaatggccgctgccccctgctggtggacagcgaggggtgggttaaggcccagtacgagggccgcctctccctgctggaggagccaggcaacggcaccttcactgtcatcctcaaccagctcaccagccgggacgccggcttctactggtgtctgaccaacggcgatactctctggaggaccaccgtggagatcaagattatcgaaggagaaccaaacctcaaggttcccgggaatgtcacggctgtgctgggagagactctcaaggtcccctgtcactttccatgcaaattctcctcgtacgagaaatactggtgcaagtggaataacacgggctgccaggccctgcccagccaagacgaaggccccagcaaggccttcgtgaactgtgacgagaacagccggcttgtctccctgaccctgaacctggtgaccagggctgatgagggctggtactggtgtggagtgaagcagggccacttctatggagagactgcagccgtctatgtggcagttgaagagaggaaggcagcggggtcccgcgatgtcagcctagcgaaggcagacgctgctcctgatgagaaggtgctagactctggttttcgggagattgagaacaaagccattcaggatcccaggctttttgcagagtgaattc

SEQ ID NO: 13 - DNA, 4465 bp, Artificial Sequence
  Description of Artificial Sequence: Expression-type plasmid pBMSP-1
  modified_base (2150): a, c, t or g
  modified_base (2214)..(2215): a, c, t or g
ctggccggcgccagatctggggaacctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgattgaaggagccactcagccgatctgaattaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgagctccaccgcggtggcggccgctctagaactagtggatcccccgggctgcaggaattcgatcagatctgatcaagcttatcgataccgtcgacctcgagggggggcccggtacccctagagtcgatttggtgtatcgagattggttatgaaattcagatgctagtgtaatgtattggtaatttgggaagatataataggaagcaaggctatttatccatttctgaaaaggcgaaatggcgtcaccgcgagcgtcacgcgcattccgttcttgctgtaaagcgttgtttggtacacttttgactagcgaggcttggcgtgtcagcgtatctattcaaaagtcgttaatggctgcggatcaagaaaaagttggaatagaaacagaatacccgcgaaattcaggcccggttgccatgtcctacacgccgaaataaacgaccaaattagtagaaaaataaaaactgactcggatacttacgtcacgtcttgcgcactgatttgaaaaatctccctcgatcgagaaagagatcaatgttgagctgcttcaaaagcaatgggattgaccagctcgcggatcctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatccgcgagcttatcgcgataccgtcgaatataataattatatttgtaagaatattattataataatataaaatatatatatataaattataatatattaattattgttaattattaataatatatatattaatcatttagatatataattctatagccttagactcctcatcaatagaagactacgtataaaaataatcagataacatctaaaacatgtagataaataatagttgtttcatatccaacatgatgtccagagcttcacgctgccgcaagcactcagggcgcaagggctgctaaaggaagcggaacacgtagaaagccagtccgcagaancggtgctgaccccggatgaatgtcagctactgggctatctggacaagggaaaacgcaagcgcannagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccagatctggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctgaggatcccccgatgagctaagctagctatatcatcaatttatgtattacacataatatcgcactcagtctttcatctacggcaatgtaccagctgatataatcagttattgaaatatttctgaatttaaacttgcatcaataaatttatgtttttgcttggactataatacctgacttgttattttatcaataaatatttaaactatatttctttcaagatgggaattaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcag

SEQ ID NO: 14 - DNA, 8074 bp, Artificial Sequence
  Description of Artificial Sequence: Expression-type plasmid pBMSP-1spJSC
  modified_base (2315): a, c, t or g
ctgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattaattcccatcttgaaagaaatatagtttaaatatttattgataaaataacaagtcaggtattatagtccaagcaaaaacataaatttattgatgcaagtttaaattcagaaatatttcaataactgattatatcagctggtacattgccgtagatgaaagactgagtgcgatattatgtgtaatacataaattgatgatatagctagcttagctcatcgggggatcctcagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatctggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgnttctgcggactggctttctacgtgttccgcttcctttagcagcccttgcgccctgagtgcttgcggcagcgtgaagctctggacatcatgttggatatgaaacaactattatttatctacatgttttagatgttatctgattatttttatacgtagtcttctattgatgaggagtctaaggctatagaattatatatctaaatgattaatatatatattattaataattaacaataattaatatattataatttatatatatatattttatattattataataatattcttacaaatataattattatattcgacggtatcgcgataagctcgcggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccgcgagctggtcaatcccattgcttttgaagcagctcaacattgatctctttctcgatcgagggagatttttcaaatcagtgcgcaagacgtgacgtaagtatccgagtcagtttttatttttctactaatttggtcgtttatttcggcgtgtaggacatggcaaccgggcctgaatttcgcgggtattctgtttctattccaactttttcttgatccgcagccattaacgacttttgaatagatacgctgacacgccaagcctcgctagtcaaaagtgtaccaaacaacgctttacagcaagaacggaatgcgcgtgacgctcgcggtgacgccatttcgccttttcagaaatggataaatagccttgcttcctattatatcttcccaaattaccaatacattacactagcatctgaatttcataaccaatctcgatacaccaaatcgactctaggggtaccatggtgctcttcgtgctcacctgcctgctggcggtcttcccagccatctccacgaagagtcccatatttggtcccgaggaggtgaatagtgtggaaggtaactcagtgtccatcacgtgctactacccacccacctctgtcaaccggcacacccggaagtactggtgccggcagggagctagaggtggctgcataaccctcatctcctcggagggctacgtctccagcaaatatgcaggcagggctaacctcaccaacttcccggagaacggcacatttgtggtgaacattgcccagctgagccaggatgactccgggcgctacaagtgtggcctgggcatcaatagccgaggcctgtcctttgatgtcagcctggaggtcagccagggtcctgggctcctaaatgacactaaagtctacacagtggacctgggcagaacggtgaccatcaactgccctttcaagactgagaatgctcaaaagaggaagtccttgtacaagcagataggcctgtaccctgtgctggtcatcgactccagtggttatgtgaatcccaactatacaggaagaatacgccttgatattcagggtactggccagttactgttcagcgttgtcatcaaccaactcaggctcagcgatgctgggcagtatctctgccaggctggggatgattccaatagtaataagaagaatgctgacctccaagtgctaaagcccgagcccgagctggtttatgaagacctgaggggctcagtgaccttccactgtgccctgggccctgaggtggcaaacgtggccaaatttctgtgccgacagagcagtggggaaaactgtgacgtggtcgtcaacaccctggggaagagggccccagcctttgagggcaggatcctgctcaacccccaggacaaggatggctcattcagtgtggtgatcacaggcctgaggaaggaggatgcagggcgctacctgtgtggagcccattcggatggtcagctgcaggaaggctcgcctatccaggcctggcaactcttcgtcaatgaggagtccacgattccccgcagccccactgtggtgaagggggtggcaggaagctctgtggccgtgctctgcccctacaaccgtaaggaaagcaaaagcatcaagtactggtgtctctgggaaggggcccagaatggccgctgccccctgctggtggacagcgaggggtgggttaaggcccagtacgagggccgcctctccctgctggaggagccaggcaacggcaccttcactgtcatcctcaaccagctcaccagccgggacgccggcttctactggtgtctgaccaacggcgatactctctggaggaccaccgtggagatcaagattatcgaaggagaaccaaacctcaaggttcccgggaatgtcacggctgtgctgggagagactctcaaggtcccctgtcactttccatgcaaattctcctcgtacgagaaatactggtgcaagtggaataacacgggctgccaggccctgcccagccaagacgaaggccccagcaaggccttcgtgaactgtgacgagaacagccggcttgtctccctgaccctgaacctggtgaccagggctgatgagggctggtactggtgtggagtgaagcagggccacttctatggagagactgcagccgtctatgtggcagttgaagagaggaaggcagcggggtcccgcgatgtcagcctagcgaaggcagacgctgctcctgatgagaaggtgctagactctggttttcgggagattgagaacaaagccattcaggatcccaggctttttgcagagtgaattcccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggggatccgtcgacggtatcgataaggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccgcgagctggtcaatcccattgcttttgaagcagctcaacattgatctctttctcgagggagatttttcaaatcagtgcgcaagacgtgacgtaagtatccgagtcagtttttatttttctactaatttggtcgtttatttcggcgtgtaggacatggcaaccgggcctgaatttcgcgggtattctgtttctattccaactttttcttgatccgcagccattaacgacttttgaatagatacgctgacacgccaagcctcgctagtcaaaagtgtaccaaacaacgctttacagcaagaacggaatgcgcgtgacgctcgcggtgacgccatttcgccttttcagaaatggataaatagccttgcttcctattatatcttcccaaattaccaatacattacactagcatctgaatttcataaccaatctcgatacaccaaatcgactctagaggatctaaccatgggatctaaaccttttttgtctcttctttcattgtcattgcttttgtttacatctactagtttggcacaagaagatgaaaggattgttcttgttgacaacaaatgtaagtgtgcccggattacttccaggatcatccgttcttccgaagatcctaatgaggacattgtggagagaaacatccgaattattgttcctctgaacaacagggagaatatctctgatcccacctcaccattgagaaccagatttgtgtaccatttgtctgacctctgtaaaaaatgtgatcctacagaagtggagctggataatcagatagttactgctacccagagcaatatctgtgatgaagacagtgctacagagacctgctacacttatgacagaaacaagtgctacacagctgtggtcccactcgtatatggtggtgagaccaaaatggtggaaacagccttaaccccagatgcctgctatcctgactgagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattaattcagatcggctgagtggctccttcaatcgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacaggttccccagatctggcgccggccag

SEQ ID NO: 15 - DNA, 1062 bp, Homo sapiens
gcatccccgaccagccccaaggtcttcccgctgagcctctgcagcacccagccagatgggaacgtggtcatcgcctgcctggtccagggcttcttcccccaggagccactcagtgtgacctggagcgaaagcggacagggcgtgaccgccagaaacttcccacccagccaggatgcctccggggacctgtacaccacgagcagccagctgaccctgccggccacacagtgcctagccggcaagtccgtgacatgccacgtgaagcactacacgaatcccagccaggatgtgactgtgccctgcccagttccctcaactccacctaccccatctccctcaactccacctaccccatctccctcatgctgccacccccgactgtcactgcaccgaccggccctcgaggacctgctcttaggttcagaagcgaacctcacgtgcacactgaccggcctgagagatgcctcaggtgtcaccttcacctggacgccctcaagtgggaagagcgctgttcaaggaccacctgagcgtgacctctgtggctgctacagcgtgtccagtgtcctgccgggctgtgccgagccatggaaccatgggaagaccttcacttgcactgctgcctaccccgagtccaagaccccgctaaccgccaccctctcaaaatccggaaacacattccggcccgaggtccacctgctgccgccgccgtcggaggagctggccctgaacgagctggtgacgctgacgtgcctggcacgcggcttcagccccaaggacgtgctggttcgctggctgcaggggtcacaggagctgccccgcgagaagtacctgacttgggcatcccggcaggagcccagccagggcaccaccaccttcgctgtgaccagcatactgcgcgtggcagccgaggactggaagaagggggacaccttctcctgcatggtgggccacgaggccctgccgctggccttcacacagaagaccatcgaccgcttggcgggtaaacccacccatgtcaatgtgtctgttgtcatggcggaggtggacggcacctgctactga

SEQ ID NO: 16 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 17 - DNA, 1023 bp, Homo sapiens
gcatccccgaccagccccaaggtcttcccgctgagcctcgacagcaccccccaagatgggaacgtggtcgtcgcatgcctggtccagggcttcttcccccaggagccactcagtgtgacctggagcgaaagcggacagaacgtgaccgccagaaacttcccacctagccaggatgcctccggggacctgtacaccacgagcagccagctgaccctgccggccacacagtgcccagacggcaagtccgtgacatgccacgtgaagcactacacgaatcccagccaggatgtgactgtgccctgcccagttcccccacctcccccatgctgccacccccgactgtcgctgcaccgaccggccctcgaggacctgctcttaggttcagaagcgaacctcacgtgcacactgaccggcctgagagatgcctctggtgccaccttcacctggacgccctcaagtgggaagagcgctgttcaaggaccacctgagcgtgacctctgtggctgctacagcgtgtccagtgtcctgcctggctgtgcccagccatggaaccatggggagaccttcacctgcactgctgcccaccccgagttgaagaccccactaaccgccaacatcacaaaatccggaaacacattccggcccgaggtccacctgctgccgccgccgtcggaggagctggccctgaacgagctggtgacgctgacgtgcctggcacgtggcttcagccccaaggatgtgctggttcgctggctgcaggggtcacaggagctgccccgcgagaagtacctgacttgggcatcccggcaggagcccagccagggcaccaccaccttcgctgtgaccagcatactgcgcgtggcagccgaggactggaagaagggggacaccttctcctgcatggtgggccacgaggccctgccgctggccttcacacagaagaccatcgaccgcttggcgggtaaacccacccatgtcaatgtgtctgttgtcatggcggaggtggacggcacctgctactga

SEQ ID NO: 18 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 19 - DNA, 993 bp, Homo sapiens
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 20 - protein, 330 aa, Homo sapiens [sequence omitted]

SEQ ID NO: 21 - DNA, 978 bp, Homo sapiens
gcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 22 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 23 - DNA, 1134 bp, Homo sapiens
gcttccaccaagggcccatcggtcttccccctggcgccctgctccaggagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacacctgcaacgtgaatcacaagcccagcaacaccaaggtggacaagagagttgagctcaaaaccccacttggtgacacaactcacacatgcccacggtgcccagagcccaaatcttgtgacacacctcccccgtgcccacggtgcccagagcccaaatcttgtgacacacctcccccatgcccacggtgcccagagcccaaatcttgtgacacacctcccccgtgcccaaggtgcccagcacctgaactcctgggaggaccgtcagtcttcctcttccccccaaaacccaaggatacccttatgatttcccggacccctgaggtcacgtgcgtggtggtggacgtgagccacgaagaccccgaggtccagttcaagtggtacgtggacggcgtggaggtgcataatgccaagacaaagccgcgggaggagcagtacaacagcacgttccgtgtggtcagcgtcctcaccgtcctgcaccaggactggctgaacggcaaggagtacaagtgcaaggtctccaacaaagccctcccagcccccatcgagaaaaccatctccaaaaccaaaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaatga

SEQ ID NO: 24 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 25 - DNA, 984 bp, Homo sapiens
gcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatga

SEQ ID NO: 26 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - DNA, 300 bp, Homo sapiens
taggctgcctgtgccccccacctgcctgtccacaacccagcctctggtacatccatgccctctgccctaagcctcacctgcacttttccttggatttcagagtctccaaaggcacaggcctcctccgtgcccactgcacaaccccaagcagagggcagcctcgccaaggcaaccacagccccagccaccacccgtaacacaggtgagaagccccttccctgcacactccacccccacccacctgctcattcctcagccgcctcctccaggcagcccttcataactccttgtctgagtctc

SEQ ID NO: 28 - protein, 383 aa, Homo sapiens
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDHGPM

SEQ ID NO: 29 - DNA, 300 bp, Homo sapiens
gtcattagctggatttagccattccacaatgtacacatatttcaaacattgtgttgtatatgataaacatgtataatttttgtcaattaaaaatttttaggaagaggaggagaagagaagaagaaggagaaggagaaagaggaacaagaagagagagagacaaagacaccaggttttttctgacccctgggctatcaaaacacctattgcccaataactagttggccgttggtgccctaaactattgaagcgattgctgttatgtggatgggccccggacacttagaaactcgtgacccc

SEQ ID NO: 30 - protein, 429 aa, Homo sapiens
PTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVSYLAMTPLIPQSKDENSDDYTTFDDVGSLWTTLSTFVALFILTLLYSGIVTFIKVK

SEQ ID NO: 31 - DNA, 500 bp, Homo sapiens
gaagctggggagaggagagcacagtggttaagtcagtccctgcagcccaactgctcccgaaggtccggccacagctgctctcgtttgctctcccctgcagagtgtccgagccacacccagcctcttggcgtctacctgctaacccctgcagtgcaggacctgtggctccgggacaaagccaccttcacctgcttcgtggtgggcagtgacctgaaggatgctcacctgacctgggaggtggctgggaaggtccccacagggggcgtggaggaagggctgctggagcggcacagcaacggctcccagagccagcacagccgtctgaccctgcccaggtccttgtggaacgcggggacctccgtcacctgcacactgaaccatcccagcctcccaccccagaggttgatggcgctgagagaacccggtgagcctggctcccaggtggggagacgagggtgcccacagcctgctgacccctacgcccgccccagggccatgac

SEQ ID NO: 32 - protein, 383 aa, Homo sapiens
PTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVSYVTDHGPMK

SEQ ID NO: 33 - DNA, 500 bp, Homo sapiens
ccacaggaaaggagaagggaggcaccacaccctggccggccccacttctctcccagtgcccccgtggccagagcctgacagcccccccacctccccgcagctgcgcaggcacccgtcaagctttctctgaacctgctggcctcgtctgaccctcccgaggcggcctcgtggctcctgtgtgaggtgtctggcttctcgccccccaacatcctcctgatgtggctggaggaccagcgtgaggtgaacacttctgggtttgcccccgcacgcccccctccacagcccaggagcaccacgttctgggcctggagtgtgctgcgtgtcccagccccgcccagccctcagccagccacctacacgtgtgtggtcagccacgaggactcccggactctgctcaacgccagccggagcctagaagtcagctgtgagtcacccccaggccagggttgggacggggactctgaggggggccataaggagctggaatccatactaggcag

SEQ ID NO: 34 - skipped

SEQ ID NO: 35 - protein, 26 aa, Homo sapiens
PTKAPDVFPIISGCRHPKDNSPVVLA

SEQ ID NO: 36 - DNA, 100 bp, Homo sapiens
gacacgccgattttttgttattagatgtaacagaccatggccccatgaaatgatcccggaccagatccgtccgcacccgccactcagcagctctggccga

SEQ ID NO: 37 - skipped

SEQ ID NO: 38 - DNA, 200 bp, Homo sapiens
cgctcggcccccgttcctccccagacctggccatgacccccctgatccctcagagcaaggatgagaacagcgatgactacacgacctttgatgatgtgggcagcctgtggaccaccctgtccacgtttgtggccctcttcatcctcaccctcctctacagcggcattgtcactttcatcaaggtcaggggagcggccagg

SEQ ID NO: 39 - skipped

SEQ ID NO: 40 - DNA, 100 bp, Homo sapiens
tcaggcttctagcccctgtctgaccccaggggctgtctttcaggtgaagtagccccagaagagcaggacgccctgtacctgcagagaagggaagcagcct

SEQ ID NO: 41 - skipped

SEQ ID NO: 42 - DNA, 495 bp, Homo sapiens
tttccctgcctcccgtcaccctgccgccagggcctctgccctgccctgccccttgtcctcaggtttccagcctcagactcccactgtgtctgtcttccagcacccaccaaggctccggatgtgttccccatcatatcagggtgcagacacccaaaggataacagccctgtggtcctggcatgcttgataactgggtaccacccaacgtccgtgactgtcacctggtacatggggacacagagccagccccagagaaccttccctgagatacaaagacgggacagctactacatgacaagcagccagctctccacccccctccagcagtggcgccaaggcgagtacaaatgcgtggtccagcacaccgccagcaagagtaagaaggagatcttccgctggccaggtaggtcgcaccggagatcacccagaagggccccccaggacccccagcaccttccactcagggcctgaccacaaagacagaagcaagggctg

SEQ ID NO: 43 - skipped

SEQ ID NO: 44 - DNA, 1920 bp, Homo sapiens
ggatccctgccacggggtccccagctcccccatccaggccccccaggctgatgggcgctggcctgaggctggcactgactaggttctgtcctcacagcctccacacagagcccatccgtcttccccttgacccgctgctgcaaaaacattccctccaatgccacctccgtgactctgggctgcctggccacgggctacttcccggagccggtgatggtgacctgggacacaggctccctcaacgggacaactatgaccttaccagccaccaccctcacgctctctggtcactatgccaccatcagcttgctgaccgtctcgggtgcgtgggccaagcagatgttcacctgccgtgtggcacacactccatcgtccacagactgggtcgacaacaaaaccttcagcggtaagagagggccaagctcagagaccacagttcccaggagtgccaggctgagggctggcagagtgggcaggggttgagggggtgggtgggctcaaacgtgggaacacccagcatgcctggggacccgggccaggacgtgggggcaagaggagggcacacagagctcagagaggccaacaaccctcatgaccaccagctctcccccagtctgctccagggacttcaccccgcccaccgtgaagatcttacagtcgtcctgcgacggcggcgggcacttccccccgaccatccagctcctgtgcctcgtctctgggtacaccccagggactatcaacatcacctggctggaggacgggcaggtcatggacgtggacttgtccaccgcctctaccacgcaggagggtgagctggcctccacacaaagcgagctcaccctcagccagaagcactggctgtcagaccgcacctacacctgccaggtcacctatcaaggtcacacctttgaggacagcaccaagaagtgtgcaggtacgttcccacctgccctggtggccgccacggaggccagagaagaggggcgggtgggcctcacacagccctccggtgtaccacagattccaacccgagaggggtgagcgcctacctaagccggcccagcccgttcgacctgttcatccgcaagtcgcccacgatcacctgtctggtggtggacctggcacccagcaaggggaccgtgaacctgacctggtcccgggccagtgggaagcctgtgaaccactccaccagaaaggaggagaagcagcgcaatggcacgttaaccgtcacgtccaccctgccggtgggcacccgagactggatcgagggggagacctaccagtgcagggtgacccacccccacctgcccagggccctcatgcggtccacgaccaagaccagcggtgagccatgggcaggccggggtcgtgggggaagggagggagcgagtgagcggggcccgggctgaccccacgtctggccacaggcccgcgtgctgccccggaagtctatgcgtttgcgacgccggagtggccggggagccgggacaagcgcaccctcgcctgcctgatccagaacttcatgcctgaggacatctcggtgcagtggctgcacaacgaggtgcagctcccggacgcccggcacagcacgacgcagccccgcaagaccaagggctccggcttcttcgtcttcagccgcctggaggtgaccagggccgaatgggagcagaaagatgagttcatctgccgtgcagtccatgaggcagcgagcccctcacagaccgtccagcgagcggtgtctgtaaatcccggtaaatgacgtactcctgcctccctccctcccagggctccatccagctgtgcagtggggaggactggccagaccttctgtccactgttgcaatgaccccaggaagctacccccaataaactgtgcctgctcagagccccagtacacccattcttgggagcgggcagggc

SEQ ID NO: 45 - protein, 574 aa, Homo sapiens [sequence omitted]

SEQ ID NO: 46 - DNA, 2213 bp, Homo sapiens
gctctagaactagtggatcccccgggctgcaggaattctctaaagaagcccctgggagcacagctcatcaccatggactggacctggaggttcctctttgtggtggcagcagctacaggtgtccagtcccaggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgaaaaccgggatcctggggccgtatagcagtggctggtacccgaactcggactactactactacggtatggacgtctggggccaagggaccacggtcaccgtctcctcagggagtgcatccgccccaacccttttccccctcgtctcctgtgagaattccccgtcggatacgagcagcgtggccgttggctgcctcgcacaggacttccttcccgactccatcactttctcctggaaatacaagaacaactctgacatcagcagcacccggggcttcccatcagtcctgagagggggcaagtacgcagccacctcacaggtgctgctgccttccaaggacgtcatgcagggcacagacgaacacgtggtgtgcaaagtccagcaccccaacggcaacaaagaaaagaacgtgcctcttccagtgattgctgagctgcctcccaaagtgagcgtcttcgtcccaccccgcgacggcttcttcggcaacccccgcagcaagtccaagctcatctgccaggccacgggtttcagtccccggcagattcaggtgtcctggctgcgcgaggggaagcaggtggggtctggcgtcaccacggaccaggtgcaggctgaggccaaagagtctgggcccacgacctacaaggtgaccagcacactgaccatcaaagagagcgactggctcagccagagcatgttcacctgccgcgtggatcacaggggcctgaccttccagcagaatgcgtcctccatgtgtgtccccgatcaagacacagccatccgggtcttcgccatccccccatcctttgccagcatcttcctcaccaagtccaccaagttgacctgcctggtcacagacctgaccacctatgacagcgtgaccatctcctggacccgccagaatggcgaagctgtgaaaacccacaccaacatctccgagagccaccccaatgccactttcagcgccgtgggtgaggccagcatctgcgaggatgactggaattccggggagaggttcacgtgcaccgtgacccacacagacctgccctcgccactgaagcagaccatctcccggcccaagggggtggccctgcacaggcccgatgtctacttgctgccaccagcccgggagcagctgaacctgcgggagtcggccaccatcacgtgcctggtgacgggcttctctcccgcggacgtcttcgtgcagtggatgcagagggggcagcccttgtccccggagaagtatgtgaccagcgccccaatgcctgagccccaggccccaggccggtacttcgcccacagcatcctgaccgtgtccgaagaggaatggaacacgggggagacctacacctgcgtggtggcccatgaggccctgcccaacagggtcaccgagaggaccgtggacaagtccaccgagggggaggtgagcgccgacgaggagggctttgagaacctgtgggccaccgcctccaccttcatcgtcctcttcctcctgagcctcttctacagtaccaccgtcaccttgttcaaggtgaaatgatcccaacagaagaacatcggagaccagagagaggaactcaaaggggcgctgcctccgggtctggggtcctggcctgcgtggcctgttggcacgtgtttctcttcccgcccggcctccagttgtgtgctctcacacaggcttccttctcgaccggcaggggctggctggcttgcaggccacgaggtgggctctaccccacactgctttgctgtgtatacgcttgttgccctgaaataaatatgcacattttatccatg

SEQ ID NO: 47 - protein, 627 aa, Homo sapiens [sequence omitted]

SEQ ID NO: 48 - protein, 822 aa, Artificial Sequence [sequence omitted]

SEQ ID NO: 49 - skipped

SEQ ID NO: 50 - protein, 159 aa, Homo sapiens
MENHLLFWGVLAVFIKAVHVKAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 51 - protein, 602 aa, Homo sapiens
MVLFVLTCLLAVFPAISTKSPIFGPEEVNSVEGNSVSITCYYPPTSVNRTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFCALGPEVANVAKFLCRQSSGENCDVVVNTLGKRAPAFEGRILLNPQDKDGSFSVVITGLRKEDAGRYLCGASDGQLQEGSPIQAWQLFVNEESTIPRSPTVVKGVAGSSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCFPCKFSSYEKYWCKWNNTGCQALPSQDEGPSKAFVNCDENSRLVSLTLNLVTRADEGWYWCGVKQGFYGETAAVYVAVEERKAAGSRDVSLAKADAAPDEKVLDSGFREIENKAIQDPRLFAE

SEQ ID NO: 52 - DNA, 2533 bp, Homo sapiens
ggtccaactgcaggcctgtggtgcaggagctgtgtgaccatggggctgtcaccaggcctctctgtgctgggttcctccagtatagaggagaggcagtatagaggagagggccgcgtcctcacagtgcattctgtgttccagcatccccgaccagccccaaggtcttcccgctgagcctctgcagcacccagccagatgggaacgtggtcatcgcctgcctggtccagggcttcttcccccaggagccactcagtgtgacctggagcgaaagcggacagggcgtgaccgccagaaacttcccacccagccaggatgcctccggggacctgtacaccacgagcagccagctgaccctgccggccacacagtgcctagccggcaagtccgtgacatgccacgtgaagcactacacgaatcccagccaggatgtgactgtgccctgcccaggtcagagggcaggctggggagtggggcggggccaccccgtcgtgccctgacactgcgcctgcacccgtgttccccacagggagccgccccttcactcacaccagagtggaccccgggccgagccccaggaggtggtggtggacaggccaggaggggcgaggcgggggcatggggaagtatgtgctgaccagctcaggccatctctccactccagttccctcaactccacctaccccatctccctcaactccacctaccccatctccctcatgctgccacccccgactgtcactgcaccgaccggccctcgaggacctgctcttaggttcagaagcgaacctcacgtgcacactgaccggcctgagagatgcctcaggtgtcaccttcacctggacgccctcaagtgggaagagcgctgttcaaggaccacctgagcgtgacctctgtggctgctacagcgtgtccagtgtcctgccgggctgtgccgagccatggaaccatgggaagaccttcacttgcactgctgcctaccccgagtccaagaccccgctaaccgccaccctctcaaaatccggtgggtccagaccctgctcggggccctgctcagtgctctggtttgcaaagcatattcctggcctgcctcctccctcccaatcctgggctccagtgctcatgccaagtacacagggaaactgaggcaggctgaggggccaggacacagcccggggtgcccaccagagcagaggggctctctcatcccctgcccagccccctgacctggctctctaccctccaggaaacacattccggcccgaggtccacctgctgccgccgccgtcggaggagctggccctgaacgagctggtgacgctgacgtgcctggcacgcggcttcagccccaaggacgtgctggttcgctggctgcaggggtcacaggagctgccccgcgagaagtacctgacttgggcatcccggcaggagcccagccagggcaccaccaccttcgctgtgaccagcatactgcgcgtggcagccgaggactggaagaagggggacaccttctcctgcatggtgggccacgaggccctgccgctggccttcacacagaagaccatcgaccgcttggcgggtaaacccacccatgtcaatgtgtctgttgtcatggcggaggtggacggcacctgctactgagccgcccgcctgtccccacccctgaataaactccatgctcccccaagcagccccacgcttccatccggcgcctgtctgtccatcctcagggtctcagcacttgggaaagggccagggcatggacagggaagaataccccctgccctgagcctcggggggcccctggcacccccatgagactttccaccctggtgtgagtgtgagttgtgagtgtgagagtgtgtggtgcaggaggcctcgctggtgtgagatcttaggtctgccaaggcaggcacagcccaggatgggttctgagagacgcacatgccccggacagttctgagtgagcagtggcatggccgtttgtccctgagagagccgcctctggctgtagctgggagggaatagggagggtaaaaggagcaggctagccaagaaaggcgcaggtagtggcaggagcggcgagggagtgaggggctggactccagggccccactgggaggacaagctccaggagggccccaccaccctagtgggtgggcctcaggacgtcccactgacgcatgcaggaaggggcacctccccttaaccacactgctctgtacggggcacgtgggcacaggtgcacactcacactcacatatatgcctgagccctgcaggagcggaacgttcacagcccagacccagttccagaaaagccaggggagtcccctcccaagcccccaagctcagcctgctcccctaggcccctctggcttccctgtgtttccactgtgcacagatcaggcaccaactccacagacccctcccaggcagcccctgctccctgcctggccaagtctcccatcccttcctaagcccaactaggacccaaagcatagacagggaggggccacgtggggtggcatcagaag

SEQ ID NO: 53 - DNA, 2516 bp, Homo sapiens
ggtccaaccgcaggcccatggtgcaggagctgtgtaacctatggggctgtcaccaggcctctctgtgctgggttcctccagtgtagaggagaggcaggtacagcctgtcctcctggggacatggcatgagggccgcgtcctcacagcgcattctgtgttccagcatccccgaccagccccaaggtcttcccgctgagcctcgacagcaccccccaagatgggaacgtggtcgtcgcatgcctggtccagggcttcttcccccaggagccactcagtgtgacctggagcgaaagcggacagaacgtgaccgccagaaacttcccacctagccaggatgcctccggggacctgtacaccacgagcagccagctgaccctgccggccacacagtgcccagacggcaagtccgtgacatgccacgtgaagcactacacgaatcccagccaggatgtgactgtgccctgcccaggtcagagggcaggctggggagtggggcggggccaccccgtcctgccctgacactgcgcctgcacccgtgttccccacagggagccgccccttcactcacaccagagtggaccccgggccgagccccaggaggtggtggtggacaggccaggaggggcgaggcgggggcacggggaagggcgttctgaccagctcaggccatctctccactccagttcccccacctcccccatgctgccacccccgactgtcgctgcaccgaccggccctcgaggacctgctcttaggttcagaagcgaacctcacgtgcacactgaccggcctgagagatgcctctggtgccaccttcacctggacgccctcaagtgggaagagcgctgttcaaggaccacctgagcgtgacctctgtggctgctacagcgtgtccagtgtcctgcctggctgtgcccagccatggaaccatggggagaccttcacctgcactgctgcccaccccgagttgaagaccccactaaccgccaacatcacaaaatccggtgggtccagaccctgctcggggccctgctcagtgctctggtttgcaaagcatattcccggcctgcctcctccctcccaatcctgggctccagtgctcatgccaagtacacagggaaactgaggcaggctgaggggccaggacacagcccagggtgcccaccagagcagaggggctctctcatcccctgcccagccccctgacctggctctctaccctccaggaaacacattccggcccgaggtccacctgctgccgccgccgtcggaggagctggccctgaacgagctggtgacgctgacgtgcctggcacgtggcttcagccccaaggatgtgctggttcgctggctgcaggggtcacaggagctgccccgcgagaagtacctgacttgggcatcccggcaggagcccagccagggcaccaccaccttcgctgtgaccagcatactgcgcgtggcagccgaggactggaagaagggggacaccttctcctgcatggtgggccacgaggccctgccgctggccttcacacagaagaccatcgaccgcttggcgggtaaacccacccatgtcaatgtgtctgttgtcatggcggaggtggacggcacctgctactgagccgcccgcctgtccccacccctgaataaactccatgctcccccaagcagccccacgcttccatccggcgcctgtctgtccatcctcagggtctcagcacttgggaaagggccagggcatggacagggaagaataccccctgccctgagcctcggggggcccctggcacccccatgagactttccaccctggtgtgagtgtgagttgtgagtgtgagagtgtgtggtgcaggaggcctcgctggtgtgagatcttaggtctgccaaggcaggcacagcccaggatgggttctgagagacgcacatgccccggacagttctgagtgagcagtggcatggccgtttgtccctgagagagccgcctctggctgtagctgggagggaatagggagggtaaaaggagcaggctagccaagaaaggcgcaggtagtggcaggagcggcgagggagtgaggggctggactccagggccccactgggaggacaagctccaggagggccccaccaccctagtgggtgggcctcaggacgtcccactgacgcatgcaggaaggggcacctccccttaaccacactgctctgtacggggcacgtgggcacacatgcacactcacactcacatatacgcctgagccctgcaggagtggaacgttcacagcccagacccagttccagaaaagccaggggagtcccctcccaagcccccaagctcagcctgctcccccaggcccctctggcttccctgtgtttccactgtgcacagatcaggcaccaactccacagacccctcccaggcagcccctgctccctgcctggccaagtctcccatcccttcctaagcccaactaggacccaaagcatagacagggaggggccgcgtggggtggcatcagaag

SEQ ID NO: 54 - DNA, 2009 bp, Homo sapiens
agctttctggggcaggccaggcctgaccttggctttggggcagggagggggctaaggtgaggcaggtggcgccagcaggtgcacacccaatgcccatgagcccagacactggacgctgaacctcgcggacagttaagaacccaggggcctctgcgcctgggcccagctctgtcccacaccgcggtcacatggcaccacctctcttgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttggtgagaggccagcacagggagggagggtgtctgctggaagcaggctcagcgctcctgcctggacgcatcccggctatgcagccccagtccagggcagcaaggcaggccccgtctgcctcttcacccggagcctctgcccgccccactcatgctcagggagagggtcttctggctttttcccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtcctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccggcaagccccgctccccgggctctcgcggtcgcacgaggatgcttggcacgtaccccctgtacatacttcccgggcgcccagcatggaaataaagcacccagcgctgccctgggcccctgcgagactgtgatggttctttccacgggtcaggccgagtctgaggcctgagtggcatgagggaggcagagcgggtc

SEQ ID NO: 55 - DNA, 2009 bp, Homo sapiens
agctttctggggcgagccgggcctgactttggctttggggcagggagtgggctaaggtgaggcaggtggcgccagccaggtgcacacccaatgcccgtgagcccagacactggaccctgcctggaccctcgtggatagacaagaaccgaggggcctctgcgcctgggcccagctctgtcccacaccgcggtcacatggcaccacctctcttgcagcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttggtgagaggccagctcagggagggagggtgtctgctggaagccaggctcagccctcctgcctggacgcaccccggctgtgcagccccagcccagggcagcaaggcaggccccatctgtctcctcacccggaggcctctgcccgccccactcatgctcagggagagggtcttctggctttttccaccaggctccaggcaggcacaggctgggtgcccctaccccaggcccttcacacacaggggcaggtgcttggctcagacctgccaaaagccatatccgggaggaccctgcccctgacctaagccgaccccaaaggccaaactgtccactccctcagctcggacaccttctctcctcccagatccgagtaactcccaatcttctctctgcagagcgcaaatgttgtgtcgagtgcccaccgtgcccaggtaagccagcccaggcctcgccctccagctcaaggcgggacaggtgccctagagtagcctgcatccagggacaggccccagctgggtgctgacacgtccacctccatctcttcctcagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaaggtgggacccgcggggtatgagggccacatggacagaggccggctcggcccaccctctgccctgggagtgaccgctgtgccaacctctgtccctacagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgccacggccggcaagcccccgctccccaggctctcggggtcgcgtgaggatgcttggcacgtaccccgtgtacatacttcccaggcacccagcatggaaataaagcacccagcgctgccctgggcccctgcgagactgtgatggttctttccgtgggtcaggccgagtctgaggcctgagtggcatgagggaggcagagtgggtc

SEQ ID NO: 56 - DNA, 2590 bp, Homo sapiens
agctttctggggcaggccaggcctgactttggctgggggcagggagggggctaaggtgacgcaggtggcgccagccaggcgcacacccaatgcccgtgagcccagacactggaccctgcctggaccctcgtggatagacaagaaccgaggggcctctgcgccctgggcccagctctgtcccacaccgcagtcacatggcgccatctctcttgcagcttccaccaagggcccatcggtcttccccctggcgccctgctccaggagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacacctgcaacgtgaatcacaagcccagcaacaccaaggtggacaagagagttggtgagaggccagcgcagggagggagggtgtctgctggaagccaggctcagccctcctgcctggacgcatcccggctgtgcagtcccagcccagggcagcaaggcaggccccgtctgactcctcacccggagcctctgcccgccccactcatgctcagggagagggtcttctggctttttccaccaggctccgggcaggcacaggctggatgcccctaccccaggcccttcacacacaggggcaggtgctgcgctcagagctgccaaaagccatatccaggaggaccctgcccctgacctaagcccaccccaaaggccaaactctctactcactcagctcagacaccttctctcttcccagatctgagtaactcccaatcttctctctgcagagctcaaaaccccacttggtgacacaactcacacatgcccacggtgcccaggtaagccagcccaggactcgccctccagctcaaggcgggacaagagccctagagtggcctgagtccagggacaggccccagcagggtgctgacgcatccacctccatcccagatccccgtaactcccaatcttctctctgcagagcccaaatcttgtgacacacctcccccgtgcccacggtgcccaggtaagccagcccaggcctcaccctccagctcaaggcaggacaagagccctagagtggcctgagtccagggacaggccccagcagggtgctgacgcgtccacctccatcccagatccccgtaactcccaatcttctctctgcagagcccaaatcttgtgacacacctcccccatgcccacggtgcccaggtaagccagcccaggcctcgccctccagctcaaggcgggacaagagccctagagtggcctgagtccagggacaggccccagcagggtgctgacgcatccacctccatcccagatccccgtaactcccaatcttctctctgcagagcccaaatcttgtgacacacctcccccgtgcccaaggtgcccaggtaagccagcccaggcctcgccctccagctcaaggcaggacaggtgccctagagtggcctgcatccagggacaggtcccagtcgggtgctgacacatctgcctccatctcttcctcagcacctgaactcctgggaggaccgtcagtcttcctcttccccccaaaacccaaggatacccttatgatttcccggacccctgaggtcacgtgcgtggtggtggacgtgagccacgaagaccccgaggtccagttcaagtggtacgtggacggcgtggaggtgcataatgccaagacaaagccgcgggaggagcagtacaacagcacgttccgtgtggtcagcgtcctcaccgtcctgcaccaggactggctgaacggcaaggagtacaagtgcaaggtctccaacaaagccctcccagcccccatcgagaaaaccatctccaaaaccaaaggtgggacccgcggggtatgagggccacatggacagaggccagcttgacccaccctctgccctgggagtgaccgctgtgccaacctctgtccctacaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaatgagtgcgacagccggcaagcccccgctccccgggctctcggggtcgcgcgaggatgcttggcacgtaccccgtgtacatacttcccgggcacccagcatggaaataaagcacccagcgctgccctgggcccctgtgagactgtgatggttctttccacgggtcaggccgagtctgaggcctgagtgacatgagggaggcagagcgggtc

SEQ ID NO: 57 - DNA, 2028 bp, Homo sapiens
agctttctggggcaggccgggcctgactttggctgggggcagggagggggctaaggtgacgcaggtggcgccagccaggtgcacacccaatgcccatgagcccagacactggaccctgcatggaccatcgcggatagacaagaaccgaggggcctctgcgccctgggcccagctctgtcccacaccgcggtcacatggcaccacctctcttgcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttggtgagaggccagcacagggagggagggtgtctgctggaagccaggctcagccctcctgcctggacgcaccccggctgtgcagccccagcccagggcagcaaggcatgccccatctgtctcctcacccggaggcctctgaccaccccactcatgctcagggagagggtcttctggatttttccaccaggctcccggcaccacaggctggatgcccctaccccaggccctgcgcatacagggcaggtgctgcgctcagacctgccaagagccatatccgggaggaccctgcccctgacctaagcccaccccaaaggccaaactctccactccctcagctcagacaccttctctcctcccagatctgagtaactcccaatcttctctctgcagagtccaaatatggtcccccatgcccatcatgcccaggtaagccaacccaggcctcgccctccagctcaaggcgggacaggtgccctagagtagcctgcatccagggacaggccccagccgggtgctgacgcatccacctccatctcttcctcagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaaggtgggacccacggggtgcgagggccacacggacagaggccagctcggcccaccctctgccctgggagtgaccgctgtgccaacctctgtccctacagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatgagtgccagggccggcaagcccccgctccccgggctctcggggtcgcgcgaggatgcttggcacgtaccccgtctacatacttcccaggcacccagcatggaaataaagcacccaccactgccctgggcccctgtgagactgtgatggttctttccacgggtcaggccgagtctgaggcctgagtgacatgagggaggcagagcgggtcccactgtccccacactgg

SEQ ID NO: 58 - DNA, 106 bp, Homo sapiens
tgccaccccaggactctgtcttccagcacccaccaaggctccggatgtgttccccatcatatcagggtgcagacacccaaaggataacagccctgtggtcctggca

SEQ ID NO: 59 - DNA, 1725 bp, Homo sapiens
atggactggacctggatcctcttcttggtggcagcagccacgcgagtccactcccagacgcagttggtgcagtctggggctgaggtgaggaagcctggggcatcagtgagggtctcctgcaaggcttctggatacaccttcatcgactcctatatccactggatacgacaggcccctgggcacgggcttgagtgggtgggatggatcaaccctaacagtggtggcacaaactatgctccgagatttcagggcagggtcaccatgaccagagacgcgtccttcagtacagcctacatggacctgagaagtctgagatctgacgactcggccgtgttttactgtgcgaaaagtgaccctttttggagtgattattataactttgactactcgtacactttggacgtctggggccaagggaccacggtcaccgtctcctcagcctccacacagagcccatccgtcttccccttgacccgctgctgcaaaaacattccctccaatgccacctccgtgactctgggctgcctggccacgggctacttcccggagccggtgatggtgacctgggacacaggctccctcaacgggacaactatgaccttaccagccaccaccctcacgctctctggtcactatgccaccatcagcttgctgaccgtctcgggtgcgtgggccaagcagatgttcacctgccgtgtggcacacactccatcgtccacagactgggtcgacaacaaaaccttcagcgtctgctccagggacttcaccccgcccaccgtgaagatcttacagtcgtcctgcgacggcggcgggcacttccccccgaccatccagctcctgtgcctcgtctctgggtacaccccagggactatcaacatcacctggctggaggacgggcaggtcatggacgtggacttgtccaccgcctctaccacgcaggagggtgagctggcctccacacaaagcgagctcaccctcagccagaagcactggctgtcagaccgcacctacacctgccaggtcacctatcaaggtcacacctttgaggacagcaccaagaagtgtgcagattccaacccgagaggggtgagcgcctacctaagccggcccagcccgttcgacctgttcatccgcaagtcgcccacgatcacctgtctggtggtggacctggcacccagcaaggggaccgtgaacctgacctggtcccgggccagtgggaagcctgtgaaccactccaccagaaaggaggagaagcagcgcaatggcacgttaaccgtcacgtccaccctgccggtgggcacccgagactggatcgagggggagacctaccagtgcagggtgacccacccccacctgcccagggccctcatgcggtccacgaccaagaccagcggcccgcgtgctgccccggaagtctatgcgtttgcgacgccggagtggccggggagccgggacaagcgcaccctcgcctgcctgatccagaacttcatgcctgaggacatctcggtgcagtggctgcacaacgaggtgcagctcccggacgcccggcacagcacgacgcagccccgcaagaccaagggctccggcttcttcgtcttcagccgcctggaggtgaccagggccgaatgggagcagaaagatgagttcatctgccgtgcagtccatgaggcagcgagcccctcacagaccgtccagcgagcggtgtctgtaaatcccggtaaatga

SEQ ID NO: 60 - protein, 428 aa, Homo sapiens
ASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 61 - DNA, 1884 bp, Homo sapiens
atggactggacctggaggttcctctttgtggtggcagcagctacaggtgtccagtcccaggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgaaaaccgggatcctggggccgtatagcagtggctggtacccgaactcggactactactactacggtatggacgtctggggccaagggaccacggtcaccgtctcctcagggagtgcatccgccccaacccttttccccctcgtctcctgtgagaattccccgtcggatacgagcagcgtggccgttggctgcctcgcacaggacttccttcccgactccatcactttctcctggaaatacaagaacaactctgacatcagcagcacccggggcttcccatcagtcctgagagggggcaagtacgcagccacctcacaggtgctgctgccttccaaggacgtcatgcagggcacagacgaacacgtggtgtgcaaagtccagcaccccaacggcaacaaagaaaagaacgtgcctcttccagtgattgctgagctgcctcccaaagtgagcgtcttcgtcccaccccgcgacggcttcttcggcaacccccgcagcaagtccaagctcatctgccaggccacgggtttcagtccccggcagattcaggtgtcctggctgcgcgaggggaagcaggtggggtctggcgtcaccacggaccaggtgcaggctgaggccaaagagtctgggcccacgacctacaaggtgaccagcacactgaccatcaaagagagcgactggctcagccagagcatgttcacctgccgcgtggatcacaggggcctgaccttccagcagaatgcgtcctccatgtgtgtccccgatcaagacacagccatccgggtcttcgccatccccccatcctttgccagcatcttcctcaccaagtccaccaagttgacctgcctggtcacagacctgaccacctatgacagcgtgaccatctcctggacccgccagaatggcgaagctgtgaaaacccacaccaacatctccgagagccaccccaatgccactttcagcgccgtgggtgaggccagcatctgcgaggatgactggaattccggggagaggttcacgtgcaccgtgacccacacagacctgccctcgccactgaagcagaccatctcccggcccaagggggtggccctgcacaggcccgatgtctacttgctgccaccagcccgggagcagctgaacctgcgggagtcggccaccatcacgtgcctggtgacgggcttctctcccgcggacgtcttcgtgcagtggatgcagagggggcagcccttgtccccggagaagtatgtgaccagcgccccaatgcctgagccccaggccccaggccggtacttcgcccacagcatcctgaccgtgtccgaagaggaatggaacacgggggagacctacacctgcgtggtggcccatgaggccctgcccaacagggtcaccgagaggaccgtggacaagtccaccgagggggaggtgagcgccgacgaggagggctttgagaacctgtgggccaccgcctccaccttcatcgtcctcttcctcctgagcctcttctacagtaccaccgtcaccttgttcaaggtgaaatga

SEQ ID NO: 62 - protein, 454 aa, Homo sapiens
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRSKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 63 - protein, 532 aa, Homo sapiens
MAPSSPRPALPALLVLLGALFPGPGNAQTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTREVTVNVLSPRYEIVIITVVAAAVIMGTAGLSTYLYNRQRKIKKYRLQQAQKGTPMKPNTQATPP

SEQ ID NO: 64 - protein, 275 aa, Homo sapiens
MSSFGYRTLTVALFTLICCPGSDEKVFEVHVRPKKLAVEPKGSLEVNCSTTCNQPEVGGLETSLDKILLDEQAQWKHYLVSNISHDTVLQCHFTCSGKQESMNSNVSVYQPPRQVILTLQPTLVAVGKSFTIECRVPTVEPLDSLTLFLFRGNETLHYETFGKAAPAPQEATATFNSTADREDGHRNFSCLAVLDLMSRGGNIFHKHSAPKMLEIYEPVSDSQMVIIVTVVSVLLSLFVTSVLLCFIFGQHLRQQRMGTYGVRAAWRRLPQAFRP

SEQ ID NO: 65 - protein, 547 aa, Homo sapiens
MATMVPSVLWPRACWTLLVCCLLTPGVQGQEFLLRVEPQNPVLSAGGSLFVNCSTDCPSSEKIALETSLSKELVASGMGWAAFNLSNVTGNSRILCSVYCNGSQITGSSNITVYGLPERVELAPLPPWQPVGQNFTLRCQVEGGSPRTSLTVVLLRWEEELSRQPAVEEPAEVTATVLASRDDHGAPFSCRTELDMQPQGLGLFVNTSAPRQLRTFVLPVTPPRLVAPRFLEVETSWPVDCTLDGLFPASEAQVYLALGDQMLNATVMNHGDTLTATATATARADQEGAREIVCNVTLGGERREARENLTVFSFLGPIVNLSEPTAHEGSTVTVSCMAGARVQVTLDGVPAAAPGQPAQLQLNATESDDGRSFFCSATLEVDGEFLHRNSSVQLRVLYGPKIDRATCPQHLKWKDKTRHVLQCQARGNPYPELRCLKEGSSREVPVGIPFFVNVTHNGTYQCQASSSRGKYTLVVVMDIEAGSSHFVPVFVAVLLTLGVVTIVLALMYVFREHQRSGSYHVREESTYLPLTSMQPTEAMGEEPSRAE

SEQ ID NO: 66 - protein, 577 aa, Homo sapiens
GVPEELFEVSIWPSQALVEFGQSLVVNCSTTCPDPGPSGIETFLKKTQVGKGPQWKEFLLEDVTENSILQCFFSCAGIQKDTSLGITVYQPPEQVILELQPAWVAVDEAFTVKCHVPSVAPLESLTLALLQGNQELHRKNFTSLAVASQRAEVIISVRAQKENDRCNSSCHAELDLSLQGGRLFQGSSPIRIVRIFEFSQSPHIWVSSLLEAGMAETVSCEVARVFPAKEVMFHMFLEDQELSSFLSWEGDTAWANATIRTMEAGDQELSCFASLGAMEQKTRKLVHSYNKWPGSSFFIRVLCCKHRVTGWFGCRHPCCPLLGMLSSEHESSSFSGFPPPILELKESYPLAGTDINVTCSGHVLTSPSPTLRLQGAPDLPAGEPAWLLLTAREEDDGNFSCEASLVVQGQRLMKTTVIQLHILCKPQLEESSCPGKQTWLEGMEHTLACVPKGNPAPALVCTWNGVVFDLEVPQKATNHTGTYRYTATNQLGSVSKDIAVIVQGLDEGISSTLFVIITVALGVGVITIALYLSYRPCKVDRRKLLYRQKEEDKEEESQFAVQEEKSTTHIIDSYLIE

SEQ ID NO: 67 - protein, 924 aa, Homo sapiens
MPGPSPGLRRALLGLWAALGLGLFGLSAVSQEPFWADLQPRVAFVERGGSLWLNCSTNCPRPERGGLETSLRRNGTQRGLRWLARQLVDIREPETQPVCFFRCARRTLQARGLIRTFQRPDRVELMPLPPWQPVGENFTLSCRVPGAGPRASLTLTLLRGAQELIRRSFAGEPPRARGAVLTATVLARREDHGANFSCRAELDLRPHGLGLFENSSAPRELRTFSLSPDAPRLAAPRLLEVGSERPVSCTLDGLFPASEARVYLALGDQNLSPDVTLEGDAFVATATATASAEQEGARQLICNVTLGGENRETRENVTIYSFPAPLLTLSEPSVSEGQMVTVTCAAGTQALVTLEGVPAAVPGQPAQLQLNATENDDRRSFFCDATLDVDGETLIKNRSAELRVLYAPRLDDSDCPRSWTWPEGPEQTLRCEARGNPEPSVHCARSDGGAVLALGLLGPVTRALSGTYRCKAANDQGEAVKDVTLTVEYAPALDSVGCPERITWLEGTEASLSCVAHGVPPPDVICVRSGELGAVIEGLLRVAREHAGTYRCEATNPRGSAAKNVAVTVEYGPRFEEPSCPSNWTWVEGSGRLFSCEVDGKPQPSVKCVGSGGATEGVLLPLAPPDPSPRAPRIPRVLAPGIYVCNATNRHGSVAKTVVVSAESPPEMDESTCPSHQTWLEGAEASALACAARGRPSPGVRCSREGIPWPEQQRVSREDAGTYHCVATNAHGTDSRTVTVGVEYRPVVAELAASPPGGVRPGGNFTLTCRAEAWPPAQISWRAPPGALNIGLSSNNSTLSVAGAMGSHGGEYECARTNAHGRHARRITVRVAGPWLWVAVGGAAGGAALLAAGAGLAFYVQSTACKKGEYNVQEAESSGEAVCLNGAGGGAGGAAGAEGGPEAAGGAAESPAEGEVFAIQLTSA

SEQ ID NO: 68 - protein, 406 aa, Homo sapiens
MDFGLALLLAGLLGLLLGQSLQVKPLQVEPPEPVVAVALGASRQLTCRLACADRGASVQWRGLDTSLGAVQSDTGRSVLTVRNASLSAAGTRVCVGSCGGRTFQHTVQLLVYAFPDQLTVSPAALVPGDPEVACTAHKVTPVDPNALSFSLLVGGQELEGAQALGPEVQEEEEEPQGDEDVLFRVTERWRLPPLGTPVPPALYCQATMRLPGLELSHRQAIPVLHSPTSPEPPDTTSPEPPNTTSPESPDTTSPESPDTTSQEPPDTTSQEPPDTTSQEPPDTTSPEPPDKTSPEPAPQQGSTHTPRSPGSTRTRRPEISQAGPTQGEVIPTGSSKPAGDQLPAALWTSSAVLGLLLLALPTYHLWKRCRHLAEDDTHPPASLRLLPQVSAWAGLRGTGQVGISPS

SEQ ID NO: 69 - protein, 739 aa, Homo sapiens
MPGKMVVILGASNILWIMFAASQAFKIETTPESRYLAQIGDSVSLTCSTTGCESPFFSWRTQIDSPLNGKVTNEGTTSTLTMNPVSFGNEHSYLCTATCESRKLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISPKNTVISVNPSTKLQEGGSVTMTCSSEGLPAPEIFWSKKLDNGNLQHLSGNATLTLIAMRMEDSGIYVCEGVNLIGKNRKEVELIVQEKPFTVEISPGPRIAAQIGDSVMLTCSVMGCESPSFSWRTQIDSPLSGKVRSEGTNSTLTLSPVSFENEHSYLCTVTCGHKKLEKGIQVELYSFPRDPEIEMSGGLVNGSSVTVSCKVPSVYPLDRLEIELLKGETILENIEFLEDTDMKSLENKSLEMTFIPTIEDTGKALVCQAKLHIDDMEFEPKQRQSTQTLYVNVAPRDTTVLVSPSSILEEGSSVNMTCLSQGFPAPKILWSRQLPNGELQPLSENATLTLISTKMEDSGVYLCEGINQAGRSRKEVELIIQVTPKDIKLTAFPSESVKEGDTVIISCTCGNVPETWIILKKKAETGDTVLKSIDGAYTIRKAQLKDAGVYECESKNKVGSQLRSLTLDVQGRENNKDYFSPELLVLYFASSLIIPAIGMIIYFARKANMKGSYSLVEAQKSKV

SEQ ID NO: 70 - protein, 537 aa, Mus musculus
MASTRAKPTLPLLLALVTVVIPGPGDAQVSIHPREAFLPQGGSVQVNCSSSCKEDLSLGLETQWLKDELESGPNWKLFELSEIGEDSSPLCFENCGTVQSSASATITVYSFPESVELRPLPAWQQVGKDLTLRCHVDGGAPRTQLSAVLLRGEEILSRQPVGGHPKDPKEITFTVLASRGDHGANFSCRTELDLRPQGLALFSNVSEARSLRTFDLPATIPKLDTPDLLEVGTQQKLFCSLEGLFPASEARIYLELGGQMPTQESTNSSDSVSATALVEVTEEFDRTLPLRCVLELADQILETQRTLTVYNFSAPVLTLSQLEVSEGSQVTVKCEAHSGSKVVLLSGVEPRPPTPQVQFTLNASSEDHKRSFFCSAALEVAGKFLFKNQTLELHVLYGPRLDETDCLGNWTWQEGSQQTLKCQAWGNPSPKMTCRRKADGALLPIGVVKSVKQEMNGTYVCHAFSSHGNVTRNVYLTVLYHSQNNWTIIILVPVLLVIVGLVMAASYVYNRQRKIRIYKLQKAQEEAIKLKGQAPPP

SEQ ID NO: 71 - protein, 537 aa, Mus musculus
MASTRAKPTLPLLLALVTVVIPGPGDAQVSIHPREAFLPQGGSVQVNCSSSCKEDLSLGLETQWLKDELESGPNWKLFELSEIGEDSSPLCFENCGTVQSSASATITVYSFPESVELRPLPAWQQVGKDLTLRCHVDGGAPRTQLSAVLLRGEEILSRQPVGGHPKDPKEITFTVLASRGDHGANFSCRTELDLRPQGLALFSNVSEARSLRTFDLPATIPKLDTPDLLEVGTQQKLFCSLEALFPASEARIYLELGGQMPTQESTNSSDSVSATALVEVTEEFDRTLPLRCVLELADQILETQRTLTVYNFSAPVLTLSQLEVSEGSQVTVKCEAHSGSKVVLLSGVEPRPPTPQVQFTLNASSEDHKRSFFCSAALEVAGKFLFKNQTLELHVLYGPRLDETDCLGNWTWQEGSQQTLKCQAWGNPSPKMTCRRKADGALLPIGVVKSVKQEMNGTYVCHAFSSHGNVTRNVYLTVLYHSQNNWTIIILVPVLLVIVGLVMAASYVYNRQRKIRIYKLQKAQEEAIKLKGQAPPP

SEQ ID NO: 72 - protein, 527 aa, Cricetulus griseus
MAPTRARPTPPLLLALVAVVIPGPGSAQVSIHPKEAFLPRGASMQVNCSSSCSENLSLGLETQWPKVELDHGHNWKLFELSDIGDDSKPLCFENCGPIQSSASATIVLYSFPERVELDRLPTWQPVGKNLTLRCLVDGGTPRSQLSVKLLRGGEVLHQEPVGVDSRNPKEVTVTVLASRDDHGANFSCRTELDLRPQGLALFPNVSVIRQLWTFDLPVTEPKLDTPDLLEVGTVQKVMCSLGGLFPAAEARITLELGGHTLTSKSTNHRDLVSATALVTAEMEGTQQLRCVLELADQILKAERTLSIYNFSAPVLTLSQQEVSEGSQVTVKCEAQGGAQVRLSGAPPGQVQFTLNASSEDHERIFTCSAALRVAGQELLKNQTLKLHVLYGPRLDENDCPGNWTWPEGSQQNLSCQAFGNPPPKLTCSRKTDGALLPIGEVKTVTWAMNGTYVCHAVSSHGNITREVFLKVLPKSPIWPIIIIVVILATVVFVGVLTIYIYNRQRKIRIYKLQRAQEEAMKLKVPPH

SEQ ID NO: 73 - protein, 544 aa, Bos taurus
MIASGPPPRVYWTSLIFLLLACCLLPTGAQGQTYQVRVEPKDPVVPFGEPLVVNCTLDCPGPGLISLETALSKEPHSRGLGWAAFRLTNVTGDMEILCSGICNKSQVVGFSNITVFGFPKRVELAPLPLWQPVGEELNLSCLVSGGAPRAHLSVVLLRGEEELGRQPLGKEEPAKVTFMVQPRREDHGTNFSCRSELDLRSQGLELFQNTSAPRKLQTYAMPKTAPRLVFPRFWEMETSWPVNCSLNGLFPASEAHIQLALGNQMLNATVVSHADTLTATATAKTEQEGTQEIVCNVTLGVENRETRESLVAYRFQGPNLNLSESNATEGTPVTVTCAAGPQVQVMLDGVPAAVPGQPAQLQLKATEMDDRRTFFCNATLKVHGVTLHRNRSIQLRVLYGPTIDRAKCPQRLMWKEKTMHILQCQARGNPNPQLQCLREGSKFKVPVGIPFLVLLNYSGTYSCQAASSRGTDKMLVMMDVQGRNPVTINIVLGVLAILGLVTLAAASVYVFWVQRQHDIYHLTPRSTRWRLTSTQPVTVAEELS

SEQ ID NO: 74 - protein, 537 aa, Sus scrofa
MAPGATHPGQLALLALLLPLLGALLPGLGGAEISMWPLNTIIPKGGSMKVNCSVACDGNITSFGLETHWHKTEVDHRDKWKIFELSNVENDGTLLCHAVCQGNQTQVQGNLTVYWFPEYVKLANLSWQREGQHFNLSCQVSGGAPRTNLSAVLFRGEEELFRQSVGMEEPANVTFRMLASRKDHGANFSCRTELNLQPQGLELFWNSSAPLKLQTYVLPATHPHLATPELVEVGTPVSVNCSLDGLFPASEATVHLARGDHRPPLTITHNGDSLLAKTWINGTEKEQGTQYLVCEIMLADEKVVTKKNVTFYSFPPPNLTLSEPEVSEGTTVSIECQAHGEAVVTLNEVPAEPPSQRAQLKLNVSAEDHGRSFSCSAALTVAGHVLYKNQTQVLSVLYGPRLDERDCPGNWTWPEGSHQTLTCQARGNPTPKLICRREGDGALLPTGDLGPVKREITGTYQCQATSSRGVATRVVVVNVIHNQNNMVIIIPVAAVAILGSVGVAAYIYNYQRKIQKYELQKAQENAAMKLSTPASPP

SEQ ID NO: 75 - protein, 912 aa, Oryctolagus cuniculus
MPGPSPGLRALLGFWVALGLGILRLSAVAQEPFWADLQPRVALVERGGSLWLNCSTNCPRPERGGLETSLRRNGPEGLRWRARQLVDIREPETQPVCFFRCAATLQARGLIRTFQRPDRVELVPLPPWQPVGENFTLSCRVPGAGPRGSLTLTLLRGAQELIRRSFAGEPARARGAVLTATVLARREDHGANFSCRAELDLRPQGLALFENSSAPRQLWTYALPLDSPRLLAPRVLEVDSQSLVSCTLDGLFPASEAGVHLALGDKRLNPEVTLEGDAIVATATATAEEEGIKQLVCAVTLGGERRESRENVTVYSFPAPLLTLSEPSAPEGKLVTVTCTAGARALVTLEGVPAAAPGQPAQLQFNASESDDGRSFFCDATLELDGETLSKNGSAELRVLYAPRLDDADCPRSWTWPEGPEQTLRCEARGNPTPAVHCARSDGGAVLALGLLGPVTRALAGTYRCTAANVQGEAVKDVTLTVEYAPALDSVGCPERVTWLEGTEASLSCVAHGVPPPSVSCVRFRQADVIEGLLLVAREHAGTYRCEAINARALAKNVAVTVEYGPSFEERSCPSNWTWVEGSEQLFSCEVEGKPQPSVQCVGSEGASEGLLLPLAPLNPSPSDPSVPRDLAPGIYVCNATNPLGSAVKTVVVSAESPPQMDDSTCPSDQTWLEGAEAAGPACARGRPSPRVRCSREGAPRPARPRVSREDAGTYLCVATNAHGSDSRTVTVGVEYRPVVAELAASPSGGVRPGGNFTLTCRAEAWPPAQISWRAPPGAPNIGLSSNNSTLSVPGAMGSHGGEYECEATNAHGHARRITVRVAGPWLWIAVGGAVGGAVLLAAGAGLAFYVQSTACKKGEYNVQEAESSGEAVCLNGAGGGAGSGAEGGPEAEDSAESPAGGEVFAIQLTSA

SEQ ID NO: 76 - protein, 917 aa, Mus musculus
MPGPSPGLRRALLGLWAALGLGILGISAVALEPFWADLQPRVALVEPGGSLWLNCSTNCPRPERGGLETSLRRNGTQRGLRWLARQLVDIREPETQPVCFFRCARRTLQARGLIRTFQRPDRVELVPLPSWQPVGENFTLSCRVPGAGPRASLTLTLLRGGQELIRRSFVGEPPRARGAMLTARVLARREDHRVNFSCLAELDLRPHGLGLFANSSAPRQLRTFAMPPHSPSLIAPRVLEVDSERPVTCTLDGLFPAPEAGVYLSLGDQRLNPNVTLDGDSLVATATATASAEQEGTKQLMCVVTLGGETRETQENLTVYSFPTPLLTLSEPEAPEGKMVTISCWAGARALVTLEGIPAAVPGQPAELQLNVTKNDDKRGFFCDAALDVDGETLRKNQSSELRVLYAPRLDDLDCPRSWTWPEGPEQTLHCEARGNPEPSVHCARPEGGAVLALGLLGPVTRALAGTYRCTAVNGQGQAVKDVTLTVEYAPALDSVGCPEHITWLEGTEASLSCVAPGVPPPSVSCVRSGKEEVMEGPLRVAREHAGTYRCEAINARGSAAKNVAVTVEYGPSFEELGCPSNWTWVEGSGKLFSCEVDGKPEPRVECVGSEGASEGIVLPLVSSNSGPRNSMTPGNLSPGIYLCNATNRHGSTVKTVVVSAESPPQMDESSCPSHQTWLEGAEATALACSARGRPSPRVHCSREGAARLERLQVSREDAGTYRCVATNAHGTDSRTVTVGVEYRPVVAELAASPPSVRPGGNFTLTCRAEAWPPAQISWRAPPGALNLGLSSNNSTLSVAGAMGSHGGEYECAATNAHGRHARRITVRVAGPWLWVAVGGAAGGAALLAAGAGLAFYVQSTACKKGEYNVQEAESSGEAVCLNGAGGTPGAEGGAETPGTAESPADGEVFAIQLTSS

SEQ ID NO: 77 - protein, 548 aa, Mus musculus
MKMLLLGVWTLLALIPCPGAAEELFQVSVHPNEALVEFGHSLTVNCSTTCPDPGPSGIETFLKKTQLSKGSQWKEFLLEDITEDLVLQCFFSCAGEQKDTVLAITMYQPPEQVILDLQPEWVAVDEAFTVTCHVPSVAPLQSLTLTLLQGDQELHRKDFLSLSLVSQRAEVTATVRAHRDNDRRNFSCRAELDLSPHGGGLFHGSSATKQLRIFEFSQNPQIWVPSLLEVGKAEIVSCEVTRVFPAQEAVFRMFLEDQELSPFSSWREDAAWASATIQAMETGDQELTCLVSLGPVEQKTRKPVYVYSFPPPILEIEDAYPLAGTDVNVTCSGHVLTSPSPTLRLQGSLNHSAPGKPAWLLFTAREEDDGRTLSCEASLEVQGQRLVRTTESQLHVLYKPRFQESRCPGNQIWVEGMHQMLACIPEGNPTPVLVCVWNGMIFDLDVPQKATQNHTGTYCCTATNPLGSVSKDITIIVQGLPEGISSSTIFIIIIFTLGMAVITVALYLNYQPCKGNSRKRMHRPREQSKGEESQFSDIRAEECHAHLC

SEQ ID NO: 78 - protein, 548 aa, Rattus norvegicus
MKMLLLGIWTLLALIPCPGTTEVLFQVSVHPNQALVEFGHSLTINCSTTCPDPGPSGIETFLKKTQLSKGSQWKEFLLEGITENSVLQCFFSCAGVQKDTALDITMYQPPEQVILDLQPEWVAIDEAFTVKCHVPSVAPLQSLTLTLLQGDQELHRKDFLSLSLVSQRAEVTVNVRAQRENDRHNFSCRAELDLSPHGGGLFHGSSATKQLRIFEFSQNPQILVPSLLEVGMAETMSCEVVRVFPAQEAVFRMFLEDQELSPFSSWKGDAAWASATIQAMETGDQELTCLVSVGPVEQKARKPVHVYSFPPPVLEIEDAYPQAGTDVNVTCSGHVLTSPSPTLRLQGSLNLSAPGEPAWLRFTAREEDDGRTLSCEASLVVQGQRLVKTTKIQLHVLYKPRFQESDCPGNQIWVEGMDQMLACIPEGNPIPALVCIWNGMTFDLEVPQKATQNHTGTYSCTATNSLGSVSKDIAVLVQGLHEGISSSTIFIIIIFTLGMAVITIALYLNYQPCKRNGRKRTHRQKEQNKGGERQFSDIQAEECHAHLC

SEQ ID NO: 79 - protein, 396 aa, Mus musculus
MGAPSALPLLLLLACSWAPGGANLSQDDSQPWTSDETVVAGGTVVLKCQVKDHEDSSLQWSNPAQQTLYFGEKRALRDNRIQLVSSTPHELSISISNVALADEGEYTCSIFTMPVRTAKSLVTVLGIPQKPIITGYKSSLREKETATLNCQSSGSKPAAQLTWRKGDQELHGDQTRIQEDPNGKTFTVSSSVSFQVTREDDGANIVCSVNHESLKGADRSTSQRIEVLYTPTAMIRPEPAHPREGQKLLLHCEGRGNPVPQQYVWVKEGSEPPLKMTQESALIFPFLNKSDSGTYGCTATSNMGSYTAYFTLNVNDPSPVPSSSSTYHAIIGGIVAFIVFLLLILLIFLGHYLIRHKGTYLTHEAKGSDDAPDADTAIINAEGGQSGGDDKKEYFI

SEQ ID NO: 80 - protein, 662 aa, Homo sapiens [sequence omitted]

SEQ ID NO: 81 - protein, 505 aa, Pan troglodytes
QTSVSPPKVILPRGGSVQVTCSTSCDQPDLLGIETPLPKKELLLGGNNWKVYELSNVQEDSQPMCYSNCPDGQSTAKTFLTVYWTPERVELAPLPSWQPVGKDLTLRCQVEGGAPRANLTVVLLRGEKELKREPAVGEPAEVTTTVLVERDHHGANFSCRTELDLRPQGLQLFENTSAPHQLQTFVLPATPPQLVSPRVLEVDTQGTVVCSLDGLFPVLEAQVHLALGDQRLNPTVTYGNDSFSAKASVSVTAEDEGTQRLTCAVILGNQSRETLQTVTIYSFPAPNVILTKPEVSEGTEVTVKCEAHPRAKVTLNGVPAQPVGPRVQLLLKATPEDNGRSFSCSATLEVAGQLIHKNQTRELRVLYGPRLDERDCPGNWTWPENSQQTPMCQASGNPLPELKCLKDGTFPLPVGESVTVTRDLEGTYLCRARSTQGEVTRKVTVNVLSPRYEIVIITVVAAAVIMGTAGLSTYLYNRQRKIRKYRLQQAQKGTPMKPNTQATPP

SEQ ID NO: 82 - protein, 447 aa, Mus musculus
  MOD_RES (12): Any amino acid
  MOD_RES (77)..(80): Any amino acid
  MOD_RES (145)..(147): Any amino acid
  MOD_RES (155)..(163): Any amino acid
  MOD_RES (268)..(269): Any amino acid
  MOD_RES (279): Any amino acid
  MOD_RES (282): Any amino acid
EDSQPMCYSNCXDGQSTAKTFLTVYWTPERVELAPLPSWQPVGKNLTLRCQVEGGAPRANLTVVLLRGEKELKREPXXXXPAEVTTTVLVRRDHHGANFSCRTELDLRPQGLELFENTSAPYQLQTFVLPATPPQLVSPRVLEVXXXGTVVCSLXXXXXXXXXQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPXXQLLLKATPEXNGXSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTREVTVNVLSPRYEIVIITVVAAAVIMGTAGLSTYLYNRQRKIKKYRLQQAQKGTPMKPNTQATPP

SEQ ID NO: 83 - protein, 528 aa, Canis familiaris
APALPRLPALLALLGALLPGLGGAQTSVDPAEAIILRGGSVQVNCSTSCNQTSIFGLETLLTKTEVTSGDNWVLFELTDVQEDSKLICFSNCHDETMAPIDLTVYWFPERVELAPLPRWQPVGENLTMTCQVAGGAPRTNLTVVLLRGEEELSRQPAVGEPAEVTFTVAVGREDHLANFSCRTDLDLRHRGLGLFQNSSAPRQLQTFVLPETPPRLATPPIVEVGTQWSVDCTMDGVFPASEAQVHLALAEERLHSTVLYKKDSLLATANVKANPEDEGTQQLWCEVTLGDENRRWQENVTFYSFPAPNLTLSEPEVSEWTTVTVECEAPAGVVVSLSGLPSGLAVPRAQFQLNASAADNRRSFSCSAALEVAGHMLQKNQTRELHVLYGPRLDQRDCPGNWTWEEGFHQTLKCQAWGNPVPELKCHRKGDDALLPIGDLRPVKREVAGTYLCQARSPRGEITREVVINVIYHQNNILIIILVTTIVILGTVSVAAYLYNRQRKIQKYKLQKAQEAAAMKLNTPATPP

SEQ ID NO: 84 - protein, 535 aa, Bos taurus
MALGAAPAAQLALLALLGTLLPGPGGAGISIHPSKAIIPRGDSLTVNCSNSCDQKSTFGLETVLIKEEVGRGDNWKVFQLRDVQEDIELFCYSNCHKEQTIASMNLTVYWFPEHVELAPLPLWQPVGEELNLSCLVSGGAPRAHLSVVLLRGEEELGRQPVGKGEPAKVMFTVQSRREDHGTNFSCRWELDLRSQGLELFQNTSAPRKLQTYVLPSIDPHLEVPPIVEVGSRWPVNCTLDGLFPASDAKVYLVLGDQKLESNITYDGDSVLAKAWMEENEEGTHSLKCSVTLGEVSRRTQENVTVYSFPLPTLTLSPPEVSEWTTVTVECVTRDGAVVKLNGTSAVPPGPRAQLKLNASASDHRSNFSCSAALEIAGQVVHKHQTLELHVLYGPRLDQRDCPGNWTWQEGSEQTLKCEAQGNPIPKLNCSRKGDGASLPIGDLRPVRREVAGTYLCRATSARGRVTREVVLNVLHGQNILDIVIPVVAVTLILGALGTAGYVYNYQRKIQKYELQKARKAQEEAALKLNAQSTPP

SEQ ID NO: 85 - protein, 530 aa, Ovis aries
MAPGAAPAALLALLVLLGTLLPGSGGAEISIHPPKAIIPRGGSLRVNCSISCDRKTTFGLETVLNKEEVSRGPNWKVFELSDVQEEINPLCYSNCHGEQIVASMNLTIYWFPERVELAPLPLWQPVGEELNLSCQVSGGGPRHHLSMVLLRGEEELDRQPVGKEEPAEVTFMVQPRREDHGTSFSCRWELDLRSQGLELFQNTSAPRKLQTYVLPSTDPHLEAPPVVEVGSRWPVKCTLDGLFPASDAEVYVQLGDQKLESNITYNGDSVLAEAWTEENEEGTHSLRCSVSLGEKIRRTRGSVTMYSFPLPTLTLSPPEVSEWTTVTVECVTRDGAVVRLNGVSAEPPGPRAQLKLNVSADDHGSNFSCSAALKIAGQEVHKIQTRELHVLYGPRLDQRDCLGNWTWQEGSEQTLKCAARGNPIPKLNCSRKGDGASLPIGDLRPVTREVAGTYLCWATSARGGVTREVVLNVLYGQNILDIVIPVVAVTLILGTLGTAGYIYNYQRKIQKYELQKAQKEAALKLKSTPP

SEQ ID NO: 86 - protein, 545 aa, Rattus norvegicus
MASTRARPMLPLLLVLVAVVIPGPVGAQVSIHPTEAFLPRGGSVQVNCSSSCEDENLGLGLETNWMKDELSSGHNWKLFKLSDIGEDSRPLCFENCGTTQSSASATITVYSFPERVELDPLPAWQQVGKNLILRCLVEGGAPRTQLSVVLLRGNETLSRQAVDGDPKEITFTVLASRGDHGANFSCFTELDLRPQGLSLFKNVSEVRQLRTFDLPTRVLKLDTPDLLEVGTQQKFLCSLEGLFPASEAQIYLEMGGQMLTLESTNSRDFVSATASVEVTEKLDRTLQLRCVLELADQTLEMEKTLRIYNFSAPILTLSQPEVSEGDQVTVKCEAHGGAQVVLLNSTSPRPPTSQGTSPRPPTSQIQFTLNASPEDHKRRFFCSAALEVDGKSLFKNQTLELHVLYGPHLDKKDCLGNWTWQEGSQQTLTCQPQGNPAPNLTCSRKADGVPLPIGMVKSVKREMNGTYKCRAFSSRGSITRDVHLTVLYHDQNTWVIIVGVLVLIIAGFVIVASIYTYYRQRKIRIYKLQKAQEEALKLKVQAPPP

SEQ ID NO: 87 - protein, 917 aa, Rattus norvegicus
MPGPSPGLRRTLLGLWAALGLGILGISAVALEPFWADLQPRVALVERGGSLWLNCSTNCPRPERGGLETSLRRNGTQRGLRWLARQLVDIREPETQPVCFFRCARRTLQARGLIRTFQRPDRVELVPLPPWQPVGENFTLSCRVPGAGPRASLTLTLLRGGQELIRRSFVGEPPRARGAMLTATVLARREDHRANFSCLAELDLRPHGLGLFANSSAPRQLRTFAMPPLSPSLIAPRFLEVGSERPVTCTLDGLFPAPEAGVYLSLGDQRLHPNVTLDGESLVATATATASEEQEGTKQLMCIVTLGGESRETQENLTVYSFPAPLLTLSEPEAPEGKMVTVSCWAGARALVTLEGIPAAVPGQPAELQLNVTKNDDKRGFFCDAALDVDGETLRKNQSSELRVLYAPRLDDLDCPRSWTWPEGPEQTLHCEARGNPEPSVHCARPDGGAVLALGLLGPVTRALAGTYRCTAINGQGQAVKDVTLTVEYAPALDSVGCPERITWLEGTEASLSCVAHGVPPPSVSCVRSGKEEVMEGPLRVAREHAGTYRCEAINARGSAAKNVAVTVEYGPSFEELGCPSNWTWVEGSGKLFSCEVDGKPEPRVECVGSEGASEGVVLPLVSSNSGSRNSMTPGNLSPGIYLCNATNRHGSTVKTVVVSAESPPQMDESSCPSHQTWLEGAEATALACSARGRPSPRVRCSREGAARLERLQVSREDAGTYLCVATNAHGTDSRTVTVGVEYRPVVAELAASPPSVRPGGNFTLTCRAEAWPPAQISWRAPPGALNLGLSSNNSTLSVAGAMGSHGGEYECAATNAHGRHARRITVRVAGPWLWVAVGGAAGGAALLAAGAGLAFYVQSTACKKGEYNVQEAESSGEAVCLNGAGGTPGAEGGAETPGTAESPADGEVFAIQLTSS

SEQ ID NO: 88 - protein, 151 aa, Homo sapiens
  MOD_RES (12): Any amino acid
  MOD_RES (77)..(81): Any amino acid
  MOD_RES (132): Any amino acid
  MOD_RES (145)..(147): Any amino acid
EDSQPMCYSNCXDGQSTAKTFLTVYWTPERVELAPLPSWQPVGKNLTLRCQVEGGAPRANLTVVLLRGEKELKREPXXXXXAEVTTTVLVRRDHHGANFSCRTELDLRPQGLELFENTSAPYQLQTFVLPAXPPQLVSPRVLEVXXXGTVV

SEQ ID NO: 89 - protein, 1252 aa, Rattus norvegicus
MGAKRVTVRGARTSPIHRMSSLTPLLLMGMLTSGLAESPVPTSAPRGFWALSENLTAVEGTTVKLWCGVRAPGSVVQWAKDGLLLGPNPKMPGFPRYSLEGDRAKGEFHLLIEACDLSDDAEYECQVGRSELGPELVSPKVILSILVSPKVLLLTPEAGSTVTWVAGQEYVVTCVSGDAKPAPDITFIQSGRTILDVSSNVNEGSEEKLCITEAEARVIPQSSDNGQLLVCEGSNPALDTPIKASFTMNILFPPGPPVIDWPGLNEGHVRAGENLELPCTARGGNPPATLQWLKNGKPVSTAWGTEHAQAVAHSVLVMTVRPEDHGARLSCQSYNSVSAGTQERSITLQVTFPPSAITILGSVSQSENKNVTLCCLTKSSRPRVLLRWWLGGRQLLPTDETVMDGLHGGHISMSNLTFLVRREDNGLPLTCEAFSDAFSKETFKKSLTLNVKYPAQKLWIEGPPEGQYIRTGTRVRLVCLAIGGNPDPSLIWFKDSRPVSEPRQPQEPRRVQLGSVEKSGSTFSRELVLIIGPPDNRAKFSCKAGQLSASTQLVVQFPPTNLTILANSSALRPGDALNLTCVSISSNPPVNLSWDKEGERLEDVAAKPQSAPFKGSAASRSVFLRVSSRDHGQRVTCRAHSEALRETVSSFYRFNVLYPPEFLGEQVRAVTVVEQGQVLLPVSVSANPAPEAFNWTFRGYRLSPAGGPRHRILSGGALQLWNVTRADDGFYQLHCQNSEGTAEALLKLDVHYAPTIRALRDPTEVNVGGSVDIVCTVDANPILPEMFSWERLGEEEEDLNLDDMEKVSKGSTGRLRIRQAKLSQAGAYQCIVDNGVAPAARGLVRLVVRFAPQVDQPTPLTKVAAAGDSTSSATLHCRARGVPNIDFTWTKNGVPLDLQDPRYTEHRYHQGVVHSSLLTIANVSAAQDYALFKCTATNALGSDHTNIQLVSISRPDPPLGLKVVSISPHSVGLEWKPGFDGGLPQRFQIRYEALETPGFLHVDVLPTQATTFTLTGLKPSTRYRIWLLASNALGDSGLTDKGIQVSVTTPGPDQAPEDTDHQLPTELPPGPPRLPLLPVLFAVGGLLLLSNASCVGGLLWRRRLRRLAEEISEKTEAGSEDRIRNEYEESQWTGDRDTRSSTVSTAEVDPNYYSMRDFSPQLPPTLEEVLYHQGAEGEDMAFPGHLHDEVERAYGPPGAWGPLYDEVRMDPYDLRWPEVQCEDPRGIYDQVAADMDAVEASSLPFELRGHLV

SEQ ID NO: 90 - protein, 1256 aa, Mus musculus
MGAKEVTVRGPGASPVHRTCRLIPLLLAGMLTTGLAQSPVPTSAPRGFWALSENLTVVEGSTIKLWCGVRAPGSVVQWAKDGLLLGPNPKIPGFPRYSLEGDSAKGEFHLLIEACDLSDDAEYECQVGRSELGPELVSPRVILSVLVPPKVLQLTPEAGSTVTWVAGQEYVVTCVSGGAKPAPDIIFIQGGRTVEDVSSSVNEGSEEKLFFTEAEARVTPQSSDNGQLLVCEGSNPALATPIKASFTMNILFPPGPPVIDWPGLNEGHVRAGENLELPCIARGGNPPATLQWLKNGKPVSIAWGTEHAQAVAHSVLVMTVRPEDHGARLSCQSYNSVSAETQERSITLQVTFPPSAVTILGSTSQSENKNVTLCCLTKSSRPRVLLRWWLGGRQLLPTDETVMDGLHGGHISMSNLTLLVKREDNGLSLTCEAFSDAFSKETFKKSLTLNVKYPAQKLWIEGPPEGQSIRTGTRVRLVCLAIGGNPEPSLTWLKDSRPVNDPRQSQEPRRVQLGSVEKSGSTFSRELVLIIGPPDNLAKFSCKAGQLSASTQLVVQFPPTNLTILANSSALRPGDALNLTCVSISSNPPVNLSLDKEGERLDDVAAKPQSAPFKGSAASRSVFLRVSSRDHGHRVTCRAHSEALRETVSSFYRLNVLYPPEFLGEQVRAVTVVEQGQALLPVSVSANPAPEAFNWTFRGYRLSPAGGPRHRILSGGALQLWNVTRADDGFYQLHCQNSEGTAEALLKLDVHYAPTIRALKDPTEVNVGGSVDIVCTVDANPILPEMFSWERLGEDEEELNLDDMEKMSKGSTGRLRIRQAKLSQAGAYQCIVDNGVAPAARGLVRLVVRFAPQVDHPTPLTKVAAAGDSTSSATLHCRARGVPNIDFTWTKNGVPLDLQDPRYTEHKYHQGVVHSSLLTIANVSAAQDYALFKCTATNALGSDHTNIQLVSISRPDPPLGLKVVSVSPHSVGLEWKPGFDGGLPQRFQIRYEALETPGFLYMDVLPAQATTFTLTGLKPSTRYRIWLLASNALGDSGLTDKGIQVSITTPGLDQAPEDTDQPLPTEQPPGPPRLPLLPVLFAVGGLLLLSNASCVGGLLWRRRLRRLAEEISEKTEAGSEEDRIRNEYEESQWTGDRDTRSSTVSTAEVDPHYYSMRDFSPQLPPTLEEVSYRQAFTGIEDEDMAFPGHLYDEVERVYGPPGVWGPLYDEVQMDPYDLRWPEVKYEDPRGIYDQVAADMDAGEPGSLPFELRGHLV

SEQ ID NO: 91 - DNA, 29 bp, Artificial Sequence
  Description of Artificial Sequence: Primer
gacctgtacttcattttggacaaatcagg

SEQ ID NO: 92 - DNA, 34 bp, Artificial Sequence
  Description of Artificial Sequence: Primer
gagctcaaaattgagtggatgatgccttgcagag

SEQ ID NO: 93 - DNA, 27 bp, Artificial Sequence
  Description of Artificial Sequence: Primer
ggtaccacttctctcaatccaactttc

SEQ ID NO: 94 - DNA, 49 bp, Artificial Sequence
  Description of Artificial Sequence: Primer
gtccaaaatgaagtacaggtcagccaaactagtagaggtgaacaaaagc

SEQ ID NO: 95 - DNA, 28 bp, Artificial Sequence
  Description of Artificial Sequence: Primer
gggggacgcagggaggatgggggtccag

SEQ ID NO: 96 - DNA, 30 bp, Artificial Sequence
  Description of Artificial Sequence: Primer
gagctcccgtcagaacagtgtgtggtggtg

SEQ ID NO: 97 - DNA, 46 bp, Artificial Sequence
  Description of Artificial Sequence: Primer
atcctccctgcgtcccccagccaaactagtagaggtgaacaaaagc

SEQ ID NO: 98 - DNA, 1650 bp, Unknown Organism
  CDS (34)..(1647)
  Description of Unknown Organism: ATR-IgA2 fusion nucleotide
ggtaccacttctctcaatccaactttctaaacaatggcttctaaacctttcttgtctcttctttctttgtctttgcttttgttcacctctactagtttggctgacctgtacttcattttggacaaatcaggaagtgtgctgcaccactggaatgaaatctattactttgtggaacagttggctcacaaattcatcagcccacagttgagaatgtcctttattgttttctccacccgaggaacaaccttaatgaaactgacagaagacagagaacaaatccgtcaaggcctagaagaactccagaaagttctgccaggaggagacacttacatgcatgaaggatttgaaagggccagtgagcagatttattatgaaaacagacaagggtacaggacagccagcgtcatcattgctttgactgatggagaactccatgaagatctctttttctattcagagagggaggctaataggtctcgagatcttggtgcaattgtttactgtgttggtgtgaaagatttcaatgagacacagctggcccggattgcggacagtaaggatcatgtgtttcccgtgaatgacggctttcaggctctgcaaggcatcatccactcaattttgagctctgcttccccaaccagccctaaggtcttccctctcagccttgacagcacccctcaagatggtaatgttgtcgttgcttgccttgtccagggtttcttccctcaggagccactctctgttacctggtctgaatctggacagaatgttaccgccagaaacttcccacctagccaggatgcctccggtgacctctacaccaccagctctcagctcacccttccagccacccagtgcccagatggtaagtccgttacctgccatgttaagcactacaccaactccagccaggatgttactgttccatgccgtgttccaccacctccaccatgctgccacccacgtctctctcttcaccgtcctgcccttgaggacttgctcttgggttctgaagctaacctcacctgcaccctcaccggtctcagagatgcctctggtgccaccttcacctggaccccaagctctggtaagagcgctgttcaaggaccacctgagcgtgacctctgtggatgctactctgttagctctgttcttcctggttgtgcccagccttggaaccacggtgagaccttcacctgcactgctgcccacccagagttgaagaccccacttaccgccaacatcaccaagtccggaaacaccttccgtcccgaggtccacctcttgccaccaccatctgaggagcttgccctcaatgagcttgttaccctcacctgccttgctcgtggattcagcccaaaggatgttcttgttaggtggcttcagggatctcaggagcttccacgtgagaagtacctcacttgggcttcccgtcaggagccaagccagggaactaccacctacgctgttaccagcatccttcgtgttgctgctgaggactggaagaagggtgagaccttctcctgcatggttggtcacgaggcccttccacttgccttcacccagaagaccattgatcgtttggctggaaagccaacccacatcaatgtttctgttgtcatggctgaggctgatggaacctgctactaa

SEQ ID NO: 99 - protein, 538 aa, Unknown Organism
  Description of Unknown Organism: ATR-IgA2 fusion amino acid
MASKPFLSLLSLSLLLFTSTSLADLYFILDKSGSVLHHWNEIYYFVEQLAHKFISPQLRMSFIVFSTRGTTLMKLTEDREQIRQGLEELQKVLPGGDTYMHEGFERASEQIYYENRQGYRTASVIIALTDGELHEDLFFYSEREANRSRDLGAIVYCVGVKDFNETQLARIADSKDHVFPVNDGFQALQGIIHSILSSASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRLAGKPTHINVSVVMAEADGTCY

SEQ ID NO: 100 - DNA, 6602 bp, Unknown Organism
  Description of Unknown Organism: pGPTV-kan-ocs-ATR-IgA2
ctggccggcgccagatctggggaacctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgaccccgccgatgacgcgggacaagccgttttacgtttggaactgacagaaccgcaacgttgaaggagccactcagccgatctgaattcactgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttgagctcggcgcgccagctggacatcatgttggatatgaaacaactattatttatctacatgttttagatgttatctgattatttttatacgtagtcttctattgatgaggagtctaaggctatagaattatatatctaaatgattaatatatatattattaataattaacaataattaatatattataatttatatatatatattttatattattataataatattcttacaaatataattattatattcgacggtatcggggcaattgtattcgacggtatcgcgataagctcgcggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccgcgagctggtcaatcccattgcttttgaagcagctcaacattgatctctttctcgatcgagggagatttttcaaatcagtgcgcaagacgtgacgtaagtatccgagtcagtttttatttttctactaatttggtcgtttatttcggcgtgtaggacatggcaaccgggcctgaatttcgcgggtattctgtttctattccaactttttcttgatccgcagccattaacgacttttgaatagatacgctgacacgccaagcctcgctagtcaaaagtgtaccaaacaacgctttacagcaagaacggaatgcgcgtgacgctcgcggtgacgccatttcgccttttcagaaatggataaatagccttgcttcctattatatcttcccttaattaaggtaccacttctctcaatccaactttctaaacaatggcttctaaacctttcttgtctcttctttctttgtctttgcttttgttcacctctactagtttggctgacctgtacttcattttggacaaatcaggaagtgtgctgcaccactggaatgaaatctattactttgtggaacagttggctcacaaattcatcagcccacagttgagaatgtcctttattgttttctccacccgaggaacaaccttaatgaaactgacagaagacagagaacaaatccgtcaaggcctagaagaactccagaaagttctgccaggaggagacacttacatgcatgaaggatttgaaagggccagtgagcagatttattatgaaaacagacaagggtacaggacagccagcgtcatcattgctttgactgatggagaactccatgaagatctctttttctattcagagagggaggctaataggtctcgagatcttggtgcaattgtttactgtgttggtgtgaaagatttcaatgagacacagctggcccggattgcggacagtaaggatcatgtgtttcccgtgaatgacggctttcaggctctgcaaggcatcatccactcaattttgagctctgcttccccaaccagccctaaggtcttccctctcagccttgacagcacccctcaagatggtaatgttgtcgttgcttgccttgtccagggtttcttccctcaggagccactctctgttacctggtctgaatctggacagaatgttaccgccagaaacttcccacctagccaggatgcctccggtgacctctacaccaccagctctcagctcacccttccagccacccagtgcccagatggtaagtccgttacctgccatgttaagcactacaccaactccagccaggatgttactgttccatgccgtgttccaccacctccaccatgctgccacccacgtctctctcttcaccgtcctgcccttgaggacttgctcttgggttctgaagctaacctcacctgcaccctcaccggtctcagagatgcctctggtgccaccttcacctggaccccaagctctggtaagagcgctgttcaaggaccacctgagcgtgacctctgtggatgctactctgttagctctgttcttcctggttgtgcccagccttggaaccacggtgagaccttcacctgcactgctgcccacccagagttgaagaccccacttaccgccaacatcaccaagtccggaaacaccttccgtcccgaggtccacctcttgccaccaccatctgaggagcttgccctcaatgagcttgttaccctcacctgccttgctcgtggattcagcccaaaggatgttcttgttaggtggcttcagggatctcaggagcttccacgtgagaagtacctcacttgggcttcccgtcaggagccaagccagggaactaccacctacgctgttaccagcatccttcgtgttgctgctgaggactggaagaagggtgagaccttctcctgcatggttggtcacgaggcccttccacttgccttcacccagaagaccattgatcgtttggctggaaagccaacccacatcaatgtttctgttgtcatggctgaggctgatggaacctgctactaagatctgtgaattcctgcagcccgggggatccactagttctagctagagcggccgccaccgcggtggcgaattaacagaggtggatggacagacccgttcttacaccggactgggcgcgggataggatattcagattgggatgggattgagcttaaagccggcgctgagaccatgctcaaggtaggcaatgtcctcagcgtcgagcccggcatctatgtcgagggcattggtggagcgcgcttcggggataccgtgcttgtaactgagaccggatatgaggccctcactccgcttgatcttggcaaagatatttgacgcatttattagtatgtgttaattttcatttgcagtgcagtattttctattcgatctttatgtaattcgttacaattaataaatattcaaatcagattattgactgtcatttgtatcaaatcgtgtttaatggatatttttattataatattgatgataattcactggccgtcgttttacaacgtcgtgactgggaaaaccctggcgttacccaacttaatcgccttgcagcacatccccctttcgccagctggcgcgccaagcttcacgctgccgcaagcactcagggcgcaagggctgctaaaggaagcggaacacgtagaaagccagtccgcagaaacggtgctgaccccggatgaatgtcagctactggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccagatctggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctgaggatcccccgatgagctaagctagctatatcatcaatttatgtattacacataatatcgcactcagtctttcatctacggcaatgtaccagctgatataatcagttattgaaatatttctgaatttaaacttgcatcaataaatttatgtttttgcttggactataatacctgacttgttattttatcaataaatatttaaactatatttctttcaagatgggaattaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcag

SEQ ID NO: 101 - DNA, 7129 bp, Unknown Organism
  Description of Unknown Organism: pGPTV-hpt-ocs-35SJ/SC
ctgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattaattcccatcttgaaagaaatatagtttaaatatttattgataaaataacaagtcaggtattatagtccaagcaaaaacataaatttattgatgcaagtttaaattcagaaatatttcaataactgattatatcagctggtacattgccgtagatgaaagactgagtgcgatattatgtgtaatacataaattgatgatatagctagcttagctcatcgggggatcccggtcggcatctactctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaatgccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatcttattgcccccctagagtcgagatctggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagccagtagctgacattcatccggggtcagcaccgtttctgcggactggctttctacgtgttccgcttcctttagcagcccttgcgccctgagtgcttgcggcagcgtgaagcttggcgcgccagctggacatcatgttggatatgaaacaactattatttatctacatgttttagatgttatctgattatttttatacgtagtcttctattgatgaggagtctaaggctatagaattatatatctaaatgattaatatatatattattaataattaacaataattaatatattataatttatatatatatattttatattattataataatattcttacaaatataattattatattcgacggtatcggggcaattgattcccgatcctatctgtcacttcatcaaaaggacagtagaaaaggaaggtggcacctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaggtaccatggtgctcttcgtgctcacctgcctgctggcggtcttcccagccatctccacgaagagtcccatatttggtcccgaggaggtgaatagtgtggaaggtaactcagtgtccatcacgtgctactacccacccacctctgtcaaccggcacacccggaagtactggtgccggcagggagctagaggtggctgcataaccctcatctcctcggagggctacgtctccagcaaatatgcaggcagggctaacctcaccaacttcccggagaacggcacatttgtggtgaacattgcccagctgagccaggatgactccgggcgctacaagtgtggcctgggcatcaatagccgaggcctgtcctttgatgtcagcctggaggtcagccagggtcctgggctcctaaatgacactaaagtctacacagtggacctgggcagaacggtgaccatcaactgccctttcaagactgagaatgctcaaaagaggaagtccttgtacaagcagataggcctgtaccctgtgctggtcatcgactccagtggttatgtgaatcccaactatacaggaagaatacgccttgatattcagggtactggccagttactgttcagcgttgtcatcaaccaactcaggctcagcgatgctgggcagtatctctgccaggctggggatgattccaatagtaataagaagaatgctgacctccaagtgctaaagcccgagcccgagctggtttatgaagacctgaggggctcagtgaccttccactgtgccctgggccctgaggtggcaaacgtggccaaatttctgtgccgacagagcagtggggaaaactgtgacgtggtcgtcaacaccctggggaagagggccccagcctttgagggcaggatcctgctcaacccccaggacaaggatggctcattcagtgtggtgatcacaggcctgaggaaggaggatgcagggcgctacctgtgtggagcccattcggatggtcagctgcaggaaggctcgcctatccaggcctggcaactcttcgtcaatgaggagtccacgattccccgcagccccactgtggtgaagggggtggcaggaagctctgtggccgtgctctgcccctacaaccgtaaggaaagcaaaagcatcaagtactggtgtctctgggaaggggcccagaatggccgctgccccctgctggtggacagcgaggggtgggttaaggcccagtacgagggccgcctctccctgctggaggagccaggcaacggcaccttcactgtcatcctcaaccagctcaccagccgggacgccggcttctactggtgtctgaccaacggcgatactctctggaggaccaccgtggagatcaagattatcgaaggagaaccaaacctcaaggttcccgggaatgtcacggctgtgctgggagagactctcaaggtcccctgtcactttccatgcaaattctcctcgtacgagaaatactggtgcaagtggaataacacgggctgccaggccctgcccagccaagacgaaggccccagcaaggccttcgtgaactgtgacgagaacagccggcttgtctccctgaccctgaacctggtgaccagggctgatgagggctggtactggtgtggagtgaagcagggccacttctatggagagactgcagccgtctatgtggcagttgaagagaggaaggcagcggggtcccgcgatgtcagcctagcgaaggcagacgctgctcctgatgagaaggtgctagactctggttttcgggagattgagaacaaagccattcaggatcccaggctttttgcagagtgaattcgttcgtatcatcggtttcgacaacgttcgtcaagttcaatgcatcagtttcattgcgcacacaccagaatcctactgagttcgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtcgacggtatcgatattaattcccgatcctatctgtcacttcatcaaaaggacagtagaaaaggaaggtggcacctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctagagtaccatggagaaccatttgcttttctggggagtcctggcggtttttattaaggctgttcatgtgaaagcccaagaagatgaaaggattgttcttgttgacaacaaatgtaagtgtgcccggattacttccaggatcatccgttcttccgaagatcctaatgaggacattgtggagagaaacatccgaattattgttcctctgaacaacagggagaatatctctgatcccacctcaccattgagaaccagatttgtgtaccatttgtctgacctctgtaaaaaatgtgatcctacagaagtggagctggataatcagatagttactgctacccagagcaatatctgtgatgaagacagtgctacagagacctgctacacttatgacagaaacaagtgctacacagctgtggtcccactcgtatatggtggtgagaccaaaatggtggaaacagccttaaccccagatgcctgctatcctgactgaatccgcggcgatgagctaagctagctatatcatcaatttatgtattacacataatatcgcactcagtctttcatctacggcaatgtaccagctgatataatcagttattgaaatatttctgaatttaaacttgcatcaataaatttatgtttttgcttggactataatacctgacttgttattttatcaataaatatttaaactatatttctttcaagagctcaaaattggatttgtaataataaaacgcaattgtttgttattgtggcgctctatcatagatgtcgctataaacctattcagcacaatatattgttttcattttaatattgtacatataagtagtagggtacaatcagtaaattgaacggagaatattattcataaaaatacgatagtaacgggtgatatattcattagaatgaaccgaaaccggcggtaaggatctgagctacacatgctcaggttttttacaacgtgcacaacagaattgaaagcaaatatcatgcgatcataggcgtctcgcatatctcattaaagcagtgaattcagatcggctgagtggctccttcaacgttgcggttctgtcagttccaaacgtaaaacggcttgtcccgcgtcatcggcggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacaggttccccagatctggcgccggccag

(19) United States
(12) Patent Application Publication
(10) Pub. No.: US 2006/0015969 A1
(43) Pub. Date: Jan. 19, 2006

(54) Novel immunoadhesins for treating and prventing toxicity and pathogen-mediated diseases

(75) Inventors: James W. Larrick, Woodside, CA (US); Keith L. Wycoff, Palo Alto, CA (US)

Correspondence Address: MORRISON & FOERSTER LLP, 425 MARKET STREET, SAN FRANCISCO, CA, 94105-2482, US

(73) Assignee: Planet Biotechnology, Inc., Hayward, CA (US)

(21) Appl. No.: 10/493,909

(22) PCT Filed: Oct. 25, 2002
(86) PCT No.: PCT/US02/34197

§ 371 Date: Jul. 21, 2005

Related U.S. Application Data
(63) Continuation-in-part of application No. 10/047,542, filed on Oct. 26, 2001, now abandoned.
Application No. 10/047,542 is a continuation-in-part of application No. PCT/US01/13932, filed on Apr. 28, 2001.
(60) Provisional application No. 60/200,298, filed on Apr. 28, 2000.

(51) Int. Cl. A01H 1/00; C12P 21/06; A61K 39/02; C12N 15/87; C07K 14/32; C12N 5/04

(52) U.S. Cl. 800/288; 514/12; 435/69.1; 435/419; 435/468; 530/350; 424/190.1

(57) ABSTRACT

Immunoadhesins active against toxins and pathogens are described, with specific examples directed to immunoadhesins for thwarting pathogens such as anthrax and the common cold. The immunoadhesin-receptor ligand principle can be employed to counter virtually any pathogen, toxicant or toxin, including, e.g., natural and synthetic metabolic poisons.

### RELATED APPLICATIONS

This application claims priority as a continuation-in-part application of Larrick andWycoff, International Patent Application Ser. No. PCT/US01/13932, filed Apr. 28, 2001, and entitled NOVEL IMMUNOADHESIN FOR THE PREVENTION OF RHINOVIRUS INFECTION, which in turn claims priority to U.S. Provisional Application Ser. No. 60/200,298, filed Apr. 28, 2000, and entitled the same. Each of these applications is herein incorporated by reference in its entirety, including all figures, drawings, and sequence listings.

### STATEMENT REGARDING FEDERALLY SPONSORED RESEARCH OR DEVELOPMENT

Federal research support was provided in the form of an SBIR Phase I grant (R43 AI43122) and SBIR Phase II grant (2R44AI43122-02).

### FIELD OF THE INVENTION

The present invention relates to immunoadhesins, their production from plants, and their use in the treatment and prevention of toxicity and pathogen-mediated ailments such as anthrax and the common cold.

### BACKGROUND OF THE INVENTION

The common cold is generally a relatively mild disease. However, significant complications resulting from colds, such as otitis media, sinusitis and asthma exacerbations are common. Human rhinoviruses (HRV) cause up to 50% of all adult colds and 25% of colds in children (Bella and Rossmann, J Struct Biol. 128:69-74, 1999, and Sperber and Hayden, Antimicrob Agents Chemother. 32:409-19, 1988). The cost to society runs into billions of dollar per year. These small, nonenveloped RNA viruses represent a subgroup of picomavirus (Rueckert, Virology, pp. 507-548, eds. Fields, et al., Raven Press, Ltd. New York, 1990) X-ray crystallography of rhinovirus identified a capsid 300 Å in diameter (1 Å=0.1 nm) with icosahedral symmetry, constructed from sixty copies each of the viral coat proteins VP1, VP2, and VP3 (Rossmann, Nature 317:145-153, 1985). A surface depression or “canyon” on HRV was suggested as the receptor binding site (Colonno, et al., Proc Natl Acad Sci USA. 85:5449-5453, 1985; Rossmann, et al. Nature 317:145-153, 1985). Of the 102 characterized HRV serotypes, 91 (known as the major group) share as their receptor a cell surface glycoprotein known as intercellular adhesion molecule-1 (ICAM-1) (Greve, et al., Cell 56:839-847, 1989; Staunton, et al., Cell 56:849-853, 1989); the binding site is located within N-terminal domain 1 (Greve, et al., J Virol. 65:6015-6023, 1991; Staunton, et al., Cell 61:243-254, 1990).

ICAM-1 is a membrane protein with five extracellular domains, a hydrophobic transmembrane domain, and a short cytoplasmic domain. ICAM-1 is expressed on many cells important in immune and inflammatory responses, and is inducible on others (Casasnovas, et al., Proc Natl Acad Sci U S A. 95:4134-9, 1998). ICAM-1 functions as a ligand for the leukocyte integrins LFA-1 and Mac-1 (Springer, Cell 76:301-14, 1994; Staunton et al., Cell 61:243-254, 1990). On the cell surface, ICAM-1 is primarily a dimer due to association of the transmembrane domains (Miller, et al., J Exp Med. 182:1231-41, 1995; Reilly, et al J Immunol. 155:529-32, 1995).

Recombinant, soluble forms of ICAM-1 (sICAM-1) consisting of the five extracellular domains were shown to be effective in blocking rhinovirus infection of human cells in vitro (Greve, et al., J Virol. 65:6015-6023, 1991; Marlin, et al., Nature. 344:70-2, 1990). Evaluation of sICAM-1 activity against a spectrum of laboratory strains and field isolates showed that all major strains of HRV are sensitive to sICAM-1. Minor strains, which do not use ICAM as a receptor, were unaffected by sICAM- 1 (Crump et al., Antiviral Chem Chemother. 4:323-327, 1993; Ohlin, et al., Antimicrob Agents Chemother. 38:1413-5, 1994).

The anti-viral activity of soluble ICAM-1 in vitro appears to be mediated by more than one mechanism. These mechanisms include competition with cell-surface ICAM-1 for binding sites, interference with virus entry or uncoating, and direct inactivation by premature release of viral RNA and formation of empty capsids (Arruda, et al., Antimicrob Agents Chemother. 36:1186-1191, 1992; Greve, et al., J Virol. 65:6015-6023, 1991; Marlin, et al., Nature 344:70-2, 1990; Martin et al., J Virol. 67:3561-8, 1993).

The host range of HRV is restricted to primates. A recent study showed that soluble ICAM-1 was effective in preventing rhinovirus infection in chimpanzees (Huguenel, et al., Am J Respir Crit Care Med. 155:1206-10, 1997). Although chimpanzees do not show clinical symptoms, infection was demonstrated by measuring seroconversion and virus shedding. A single dose of 10 mg of soluble ICAM-1 as an intranasal spray was effective at preventing infection by HRV-16 when co-administered with HRV, or when the virus was administered ten minutes later.

A human clinical trial with soluble ICAM-1 showed that it reduced the severity of experimental HRV colds (Turner, et al., JAMA 281:1797-804, 1999). In this trial a total of 196 subjects received either soluble ICAM-1 or placebo in various formulations. Some subjects were given soluble ICAM-1 or placebo starting seven hours before inoculation with HRV 39 and others were started twelve hours after virus inoculation. Medications were administered as either an intranasal solution or powder, given in six daily doses for seven days (a total of 4.4 mg per day). In this study, soluble ICAM-1 did not prevent infection, as measured by either virus isolation or seroconversion (infection rate of 92% for placebo-treated vs. 85% of soluble ICAM-1 treated). However, soluble ICAM-1 did have an impact on all measures of illness. The total symptom score was reduced by 45%, the proportion of subjects with clinical colds was reduced 23% and nasal mucus weight was reduced by 56%. There was not a significant difference between the use of powder or solution formulations, or between pre- and post-inoculation groups. Treatment with soluble ICAM-1 did not result in any adverse effects or evidence of absorption through the nasal mucosa. Also, there was no inhibition of the development of anti-HRV type-specific antibodies.

As discussed, ICAM-1 is dimeric on the cell surface. Martin et al., in J Virol. 67:3561-8, (1993) first proposed that multivalent binding to HRV by a multimeric soluble ICAM might result in a higher effective affinity, termed avidity, and thus facilitate uncoating of the virus. They constructed multivalent, ICAM-1/immunoglobulin molecules, postulating that these would be more effective than monovalent soluble ICAM-1 in neutralizing HRV and thus would have increased therapeutic utility. These ICAM-1/immunoglobulin molecules included ICAM-1 amino-terminal domains 1 and 2 fused to the hinge and constant domains of the heavy chains of IgA1 (IC1-2D/IgA), IgM (IC1-2D/IgM) and IgG1(IC1-2D/IgG). In addition, five extracellular domains were fused to IgA1 (IC1-5D/IgA). These ICAM-1/immunoglobulin molecules were compared with soluble forms of ICAM-1 having two (sIC1-2D) and five (sIC1-5D) domains in assays of HRV binding, infectivity and conformation. The ICAM-1/IgA immunoglobulin (IC1-5D/IgA) was 200 times, and the ICAM-1/IgM immunoglobulin (IC1-2D/IgM) and ICAM-1/IgG immunoglobulin molecules (IC1-2D/IgG) were 25 and 10 times, more effective than soluble ICAM-1. These molecules were highly effective in inhibiting rhinovirus binding to cells and disrupting the conformation of the virus capsid. The ICAM-1/IgA immunoglobulin molecules were effective in the nanomolar concentration range. Comparison of IC1-2D/IgA and IC1-2D/IgG showed that the class of Ig constant region used had a large impact on efficacy.

A subsequent study compared the inhibitory activities of soluble ICAM-1 and IC1-5D/IgA against nine major HRV serotypes and a variant of HRV-39 selected for moderate resistance to soluble ICAM-1 (Crump, et al., Antimicrob Agents Chemother. 38:1425-7, 1993). IC1-5D/IgA was more potent than monomeric soluble ICAM-1 by 50 to 143 times on a weight basis and by 60 to 170 times on a molar basis against the standard serotypes. The HRV-39 variant was 38-fold more resistant to soluble ICAM-1 than the wild-type, and it was only 5-fold more resistant to IC1-5D/IgA. This is consistent with the hypothesis that virus escape from inhibition by multivalent molecules would be expected to occur at lower frequency than virus escape from inhibition by monomeric soluble receptor (Martin, et al., J Virol. 67:3561-8, 1993). An assay designed to measure viral inactivation showed that HRV-39 and HRV-13 were not directly inactivated to a significant extent by soluble ICAM-1 (<0.5 log₁₀ reduction in infectivity). However, incubation with IC1-5D/IgA resulted in a reduction of infectivity of these same viruses by about 1.0 log₁₀ (Crump, et al., Antimicrob Agents Chemother. 38:1425-7, 1994). Results by Martin et al. (J Virol. 67:3561-8, 1993) suggest that the greater the valence, the greater the effectiveness of the molecules. Dimeric and decameric forms of IC1-2/IgM were separable by sucrose gradient sedimentation. The decameric form was five times more effective than the dimeric form at blocking binding of HRV to HeLa cells.

The ICAM-1/immunoglobulin molecules that have been described suffer from several drawbacks, including the laborious production techniques and high costs associated with those production methods. In addition, the previously described ICAM-1/immunoglobulin molecules have limited stability as multimers in the harsh environment in which the molecule must inactivate rhinoviruses.

Applicants' previous, commonly owned application, International Application Ser. No. PCT/US01/13932, described the construction, purification, and use of chimeric immunoadhesin molecules, with examples and claims directed to treating or preventing viral infections and diseases, e.g., the common cold. There is a need for similar agents for the treatment and prevention of toxicity and other pathogen-mediated diseases and ailments, e.g., bacterial infections and diseases, such as anthrax. The bioterrorism scare following Sep. 11, 2001 underscores this need.

The tripartite toxin secreted by Bacillus anthracis, the causative agent of anthrax, helps the bacterium evade the immune system and can kill the host during a systemic infection. Two components of the toxin enzymatically modify substrates within the cytosol of mammalian cells: oedema factor (OF) is an adenylate cyclase that impairs host defences through a variety of mechanisms including inhibiting phagocytosis; lethal factor (LF) is a zinc-dependent protease that cleaves mitogen-activated protein kinase kinase and causes lysis of macrophages. Protective antigen (PA), the third component, binds to a cellular receptor and mediates delivery of the enzymatic components to the cytosol. After binding to the cell-surface receptor, PA is cleaved into two fragments by a furin-like protease. The amino-terminal fragment, PA₂₀, dissociates into the medium, and this allows the carboxy-terminal fragment, PA₆₃ to heptamerize and bind LF and OF. The resulting complexes of [PA₆₃]₇ with OF and/or LF are taken up into cells by receptor-mediated endocytosis and moved to a low-pH endosomal compartment. There, the acidic environment induces a conformation change in [PA₆₃]₇ that allows it to insert into the membrane and form a pore. This conversion promotes the translocation of bound OF and LF across the endosomal membrane to the cytosol.

The immunoadhesins of the present invention may be tailored to combat any pathogenic agent or poison and has significant advantages over what has been described in the art. The immunoadhesins of the present invention that are expressed in plants would be tetrameric, rather than only dimeric. Immunoadhesins having multiple binding sites have a higher effective affinity for the pathogen/toxicant, thereby increasing the effectiveness of the immunoadhesin. In addition, the association of secretory component and immunoglobulin J chain with the immunoadhesin of the present invention increases the stability of the immunoadhesin in the mucosal environment (Corthesy, Biochem Soc Trans. 25:471-475, 1997). Secretory IgA, which is associated with secretory component, is the antibody isotype normally found in mucosal secretions, including milk and colostrum. Unlike other antibody isotypes, SIGA can pass through the gut with very little proteolytic degradation. It also is very stable in crude plant preparations at room temperature. A function of the secretory component appears to be to protect the antibody from the harsh environment of the mucosa (Paul, Fundamental Immunology, Raven Press, NY, Third Edition, pp. 303-304, 1993). Furthermore, the immunoadhesins of the present invention are significantly less expensive to produce in plants than in animal cell culture, and production in plants would make it safer for human use, since plants are not known to harbor any animal viruses.

The preceding discussed documents as well as those which follow may be useful in understanding the invention but are not admitted to be prior art to the invention:
Bäumlein H, Wobus U, Pustell J, Kafatos FC (1986) The legumin gene family: structure of a B type gene of Vicia faba and a possible legumin gene specific regulatory element. Nucl. Acids Res. 14: 2707-2713
Becker D, Kemper E, Schell J, Masterson R (1992) New plant binary vectors with selectable markers located proximal to the left T-DNA border. Plant Mol. Biol. 20: 1195-1197
Bradley K A, Mogridge J, Mourez M, Collier R J, Young J A T (2001) Identification of the cellular receptor for anthrax toxin. Nature 414: pre-publication
Chintalacharuvu K R, Raines M, Morrison S L (1994) Divergence of human alpha-chain constant region gene sequences. A novel recombinant alpha 2 gene. Journal of Immunology 152: 5299-5304
Crump et al. (1994) Comparative Antirhinoviral Activities of Soluble Intercellular Adhesion Molecule-1 (sICAM-1) and Chimeric ICAM-1Immunoglobulin A Molecule. Antimicrobial Agents and Chemotherapy, 38:6, p. 1425-1427
Depicker A, Stachel S, Dhaese P, Zambryski P, Goodman H M (1982) Nopaline synthase: transcript mapping and DNA sequence. J. Mol. Appl. Genet. 1: 561-573
Gielen J, De Beuckeleer M, Seurinck J, Deboeck F, De Greve H, Lemmers M, Van Montagu M, Schell J (1984) The complete nucleotide sequence of the TL-DNA of the Agrobacterium tumefaciens plasmid pTiAch5. Embo J 3: 83546
Greve et al.(1991) EP 0468257, Multimeric form of human rhinovirus receptor protein.
Horsch R B, Fry J E, Hoffmann N L, Eichholtz D, Rogers S G, Fraley R T (1985) A simple and general method for transferring genes into plants. Science 227: 1229-1231
Ingelbrecht I, Breyne P, Vancompernolle K, Jacobs A, Van Montagu M, Depicker A (1991) Transcriptional interference in transgenic plants. Gene 109: 239-242
MacDonald M H, Mogen B D, Hunt A G (1991) Characterization of the polyadenylation signal from the T-DNA-encoded octopine synthase gene. Nucleic Acids Res 19: 5575-81
Martin et al. (1993), Efficient Neutralization and Disruption of Rhinovirus by Chimeric ICAM-1/Immunoglobulin Molecules. J. of Virology, 67:6, p. 3561-3568.
Mogen B D, MacDonald M H, Leggewie G, Hunt A G (1992) Several distinct types of sequence elements are required for efficient mRNA 3′ end formation in a pea rbcS gene. Mol Cell Biol 12: 5406-14
Ni M, Cui D, Einstein J, Narasimhulu S, Vergara C E, Gelvin S B (1995) Strength and tissue specificity of chimeric promoters derived from the octopine and mannopine synthase genes. Plant Journal 7: 661-676
Sawant S V, Singh P K, Gupta S K, Madnala R, Tuli R (1999) Conserved nucleotide sequences in highly expressed genes in plants. Journal of Genetics 78: 123-131
St Croix B, Rago C, Velculescu V, Traverso G, Romans K E, Montgomery E, Lai A, Riggins G J, Lengauer C, Vogelstein B, Kinzler K W (2000) Genes expressed in human tumor endothelium. Science 289: 1197-202.
Yamamoto Y Y, Tsuji H, Obokata J (1995) 5′-leader of a photosystem I gene in Nicotiana sylvestris, psaDb, contains a translational enhancer. J Biol Chem 270: 12466-70.

### SUMMARY OF THE INVENTION

The present invention contemplates an immunoadhesin comprising a chimeric molecule having a toxin receptor protein linked to at least a portion of an immunoglobulin heavy chain, wherein J chain and secretory component are associated with the chimeric molecule. A toxin receptor as used here in is a receptor molecule or a part of a receptor molecule, at least a portion of which is a protein or peptide found on the surface of or in the cells of a host organism, to which toxicants, e.g., poisons or pathogenic organisms such as viruses, bacteria, fungi, parasites (or a molecule produced by such pathogenic organism) etc. attach as part of the disease generating process. In a preferred embodiment the toxin receptor will be the extra-cellular domain of a receptor molecule. The toxin receptor may be glycosylated or non-glycosylated. Alterations and modifications to the receptor protein or portion thereof are also contemplated, provided such modifications do not destroy the ability of the receptor to bind the toxin, toxicant, pathogen, or pathogen component.

In some embodiments in which the receptor protein is a viral receptor protein, the immunoadhesin of the present invention is comprised of a rhinovirus receptor protein made of any combination of extracellular domains 1, 2, 3, 4 and 5 of the rhinovirus receptor protein, ICAM-1, linked to an immunoglobulin heavy chain. Also contemplated by the present invention are immunoadhesins of the present invention in which the immunoglobulin is IgA, IgA1, IgA2, IgG1, IgG2, IgG3, IgG4, IgM, IgD, IgE or a chimeric immunoglobulin heavy chain made up of domains or segments from different immunoglobulin isotypes.

In other preferred embodiments of the present invention, the immunoadhesin comprises multiple chimeric ICAM-1 molecules associated with J chain and secretory component. The increase in valency results in a higher effective affinity for the rhinovirus, thereby increasing the effectiveness of the immunoadhesin.

In a preferred embodiment of the present invention, all proteins used to make the immunoadhesin of the present invention are human proteins. In addition to production in plants or plant cells, the present invention contemplates an immunoadhesin expressed in mammalian cells, hairy root cultures, plant cells in tissue culture, and heterologous cells derived from plants, vertebrates or invertebrates.

In preferred embodiments of the present invention, the immunoadhesins are expressed, in plants, including monocotyledonous plants and dicotyledonous plants as a part of the plants genome. Expression in plants, as opposed to expression in cultured cells, allows for a significant reduction in the cost of producing the immunoadhesin.

The present invention contemplates an immunoadhesin having plant-specific glycosylation. A gene coding for a polypeptide having within its amino acid sequence, the glycosylation signal asparagine-X-serine/threonine, where X can be any amino acid residue, is glycosylated via oligosaccharides linked to the asparagine residue of the sequence when expressed in a plant cell. See Marshall, Ann. Rev. Biochem., 41:673 (1972) and Marshall, Biochem. Soc. Symp., 40:17 (1974) for a general review of the polypeptide sequences that function as glycosylation signals. These signals are recognized in both mammalian and in plant cells. At the end of their maturation, proteins expressed in plants or plant cells have a different pattern of glycosylation than do proteins expressed in other types of cells, including mammalian cells and insect cells. Detailed studies characterizing plant-specific glycosylation and comparing it with glycosylation in other cell types have been performed, for example, in studies described by Cabanes-Macheteau et al., Glycobiology 9(4):365-372 (1999), and Altmann, Glycoconjugate J. 14:643-646 (1997). These groups and others have shown that plant-specific glycosylation generates glycans that have xylose linked β(1,2) to mannose, but xylose is not linked β(1,2) to mannose as a result of glycosylation in mammalian and insect cells. Plant-specific glycosylation results in a fucose linked α(1,3) to the proximal GlcNAc, while glycosylation in mammalian cells results in a fucose linked α(1,6) to the proximal GlcNAc. Furthermore, plant-specific glycosylation does not result in the addition of a sialic acid to the terminus of the protein glycan, whereas in glycosylation in mammalian cells, sialic acid is added.

In other embodiments, the immunoadhesin of the present invention is part of a composition comprising plant material and the immunoadhesin, associated with J chain and secretory component. The plant material present may be plant cell walls, plant organelles, plant cytoplasms, intact plant cells, viable plants, and the like. The particular plant materials or plant macromolecules that may be present include ribulose bisphosphate carboxylase, light harvesting complex, pigments, secondary metabolites or chlorophyll. Compositions of the present invention may have an immunoadhesin concentration of between 0.001% and 99.9% mass excluding water. In other embodiments, the immunoadhesin is present in a concentration of 0.01% to 99% mass excluding water. In other embodiments, the compositions of the present invention have plant material or plant macromolecules present at a concentration of 0.01% to 99% mass excluding water.

The present invention also contemplates methods for the treatment or prevention of human rhinovirus infection in a subject, including reducing the infection by human rhinovirus of host cells susceptible to infection by the virus, or reducing the initiation or spread of the common cold due to human rhinovirus, by a method comprising contacting the virus with an immunoadhesin of the present invention, wherein the immunoadhesin binds to the human rhinovirus and reduces infectivity. The immunoadhesin could mediate infection by competition with cell-surface ICAM-1 for binding sites, interference with virus entry or uncoating, and/or direct inactivation by premature release of viral RNA and formation of empty capsids (Arruda, et al., Antimicrob. Agents Chemother. 36:1186-1191, 1992; Greve, et al., J. Virol. 65:6015-6023, 1991; Martin, et al., Nature 344:70-2, 1990; Martin, et al., J. Virol. 67:3561-8, 1993). In another embodiment, human rhinovirus infection in a subject is treated by a method comprising intranasally administering to the subject an effective amount of an immunoadhesin of the present invention, wherein the immunoadhesin reduces human rhinovirus infectivity.

Other aspects of the invention contemplate immunoadhesins, compositions, and methods of use thereof in which the immunoadhesins are active against a bacterium or bacteria. In such aspects, the immunoadhesins contain a receptor protein that binds a bacterium of interest, e.g., Bacillus anthracis, or a pathogenic component thereof, e.g., protective antigen (PA). In some preferred embodiments for the treatment or prevention of anthrax, that receptor protein is the Anthrax toxin receptor protein or a portion thereof. The portion can be an extracellular domain or a portion of that domain. Additional or alternative embodiments can track those already described for ICAM immunoadhesins, discussed above. For example, at least a portion of an immunoglobulin heavy chain, a J chain, and a secretory component as described above are also present in some preferred embodiments.

In another distinct aspect the invention features a method for reducing or preventing the binding of toxin or pathogen (e.g., the protective antigen (PA) of Bacillus anthracis) to host cells susceptible to damage by said toxin or pathogen by contacting the toxin or pathogen with immunoadhesins that bind to it, thereby decoying the toxin or pathogen, and masking and/or ameliorating its pathological effect. Other aspects feature methods of reducing mortality and morbity based on this concept.

In poison contexts, the invention also features poison antidotes that comprise a poison receptor or portion thereof linked to an immunoahesin.

While anthrax and the common cold are two enumerated targets in various aspects of the invention, the invention as concerns immunoadhesins generally can make use of any known receptor protein or portion thereof that can bind to a component of any pathogen or toxicant, which component is required by that pathogen to exert its pathogenic or toxic effect. In addition to natural pathogens, toxicants include but are not limited to venom, carcinogens, mutagens, or other metabolic inhibitors or accelerators that can have a negative effect on cells, tissues, organs, or organisms. The principle described herein can thus be used to prevent, treat, ameliorate, or modulate any type of toxicity or pathogen-mediated disease or symptom caused by such.

### BRIEF DESCRIPTION OF THE DRAWINGS

FIG. 1 illustrates pSSPHuA2, vector in which DNAs encoding a chimeric ICAM-1 molecule containing the first five domains of human ICAM-1 and the Fc region of human IgA2m(2) were fused [SEQ ID NO:9, 48]. This vector contains the SuperMas promoter for driving the expression of a signal peptide and the constant regions of the human IgA2m(2) heavy-chain. Sequences encoding ICAM domains 1-5 were amplified, by PCR, to contain convenient restriction sites (5′ SpeI and 3′ SpeI) for insertion between the signal peptide and the Cα1 domain. DNA encoding an ER retention signal (RSEKDEL) [SEQ ID NO:5] was appended to the 3′ end of the heavy-chain in order to boost the expression level of the construct.

FIG. 2 illustrates a chimeric ICAM molecule. 2A shows the DNA expression cassette from which the chimeric ICAM-1 molecule was produced. 2B shows the amino acid sequence, after signal peptide cleavage, of the mature form of the fusion protein [SEQ ID NO:8]. Amino acids introduced by the cloning procedure are underlined and mark the junction between the five extracellular domains of ICAM-1 and the Cα1-Cα3 domains of the IgA2m(2) heavy chain. The bolded N's indicate the fifteen potential glycosylation sites.

FIG. 3 illustrates the expression of the immunoadhesin in independently transformed tobacco calli. 3A shows immunoblots of non-reducing SDS-polyacrylamide gels on which samples containing different transformed tobacco calli (C) and aqueous extracts (Aq) were run and probed for the presence of human ICAM. The molecular weight markers are indicated, and the reference standard (R) was a mixture (˜75 ng each) of human ICAM (˜75 kD) and human SigA (>>250 kD). 3B shows immunoblots of nonreducing SDS-polyacrylamide gels containing various fractions of partially purified immunoadhesin from callus Rhi107-11. The purification fractions analyzed were juice (3), G-100 fraction (G), sterile filtered G-100 fraction (SG), and a mixture of reference standards of human SigA (75 ng) and human ICAM-1 (75ng) (RS).

Blots were probed with antibodies against human ICAM (˜ICAM), human IgA heavy chain (˜α), human secretory component (˜SC) and human J chain (˜J). Secondary, enzyme-conjugated antibodies were employed as necessary to label immuno-positive bands with alkaline phosphatase.

FIG. 4 illustrates the results of an enzyme-linked immunosorbent assay (ELISA) showing competition between plant extract and soluble ICAM-1 for binding to an anti-ICAM mAb. For the assay, 96-well plates were coated with 0.25 μg soluble ICAM-1/ml. The squares represent the increasing concentrations of sICAM and the circles represent the increasing amounts of callus extract (sterile filtered fraction from G-100) used to compete with the adhered ICAM for a constant amount of a mouse (anti-human ICAM) antibody.

FIG. 5 illustrates the results of an assay showing the ability of an immunoadhesin to inhibit human rhinovirus killing of HeLa cells (cytopathic effect, or CPE, assay). 5A shows the results of an assay comparing the CPE of human rhinovirus on HeLa cells in the presence of partially purified extracts containing either the immunoadhesin in the ICAM-Fc fusion (IC1-5D/IgA) or containing an antibody against doxorubicin. (The right side-up and upside-down triangles represent two extracts derived from Rhi107-11, containing the immunoadhesin.) 5B shows the results of an assay comparing the CPE of human rhinovirus on HeLa cells in the presence of soluble human ICAM-1 or an extract from the immunoadhesin in the ICAM-Fc fusion (IC1-5D/IgA). The Inset shows scale expansion in the range of the IC50 for soluble ICAM (1.35 μg/ml) and for IC1-5D/IgA (0.12 μg/ml; 11.3 fold-less).

FIG. 6 shows an evaluation of the production necessities for making 1 gram of finished immunoadhesin. In this diagram, the number of plants needed for 1 g of immunoadhesin, at 20% yield, at expected levels of expression and plant weight is illustrated. At different levels of immunoadhesin expression (mg/kg fresh weight) and overall recovery (set at 20%), the weight of each plant, and so the total number of plants, may be determined for a specified production target (1 g/harvest) within a window (dotted square) of reasonable possibilities. The number of required plants decreases, inversely, with the number of specified growth and re-growth periods. The expected biomass production, a function of time and growth conditions, influences the time to harvest and the time between harvests. These growth periods can be adjusted to the realities of the purification schedule by staggering planting and harvesting dates.

FIG. 7 shows the coding and amino acid sequences of each of the immunoglobulin genes and proteins listed in Table 2 [SEQ ID NO:15 through 47 and SEQ ID NO:52 through 62].

FIG. 8 shows the sequences of plasmids used to transform plants, as described in Example 2, for use in studies of the expression of immunoadhesins of the present invention.

FIG. 8A shows the nucleotide [SEQ ID NO:9] and protein [SEQ ID NO:48] sequences for plasmids PSSpICAMHuA2

FIG. 8B shows the nucleotide and protein [SEQ ID NO:10] sequence for the bean legumin signal peptide.

FIG. 8C shows the nucleotide [SEQ ID NO:11] and amino acid [SEQ ID NO:50] sequence of the protein coding region of pSHuJ.

FIG. 8D shows the nucleotide [SEQ ID NO:12] and amino acid [SEQ ID NO:51] sequence of protein coding region of pSHuSC.

FIG. 8E shows the nucleotide sequence [SEQ ID NO: 13] of plasmid pBMSP-1.

FIG. 8F shows the nucleotide sequence [SEQ ID NO: 14] of plasmid pBMSP-1spJSC.

FIG. 9 contains nucleotide and protein sequences SEQ ID NO: 1; SEQ ID NO:2; SEQ ID NO:3; SEQ ID NO:4; SEQ ID NO:5; SEQ ID NO:6; SEQ ID NO:7; SEQ ID NO:8, for ICAM-1, and human IgA2 and other nucleotide sequences.

FIG. 10 shows the full nucleotide (SEQ ID NO: 98) and amino acid sequence (SEQ ID NO: 99) of the ATR-IgA2 fusion (an immunoadhesin).

FIG. 11 shows the sequence (SEQ ID NO: 100) between the T-DNA borders of the plasmid pGPTV-kan-ocs-ATR-IgA2.

FIG. 12 shows the sequence (SEQ ID NO: 101) between the T-DNA borders of the plasmid pGPTV-hpt-ocs-35SJ/SC.

### DETAILED DESCRIPTION OF THE INVENTION

A. Definitions

As used herein, the following abbreviations and terms include, but are not necessarily limited to, the following definitions.

The practice of the present invention will employ, unless otherwise indicated, conventional techniques of immunology, molecular biology, microbiology, cell biology and recombinant DNA, which are within the skill of the art. See, e.g., Sambrook, et al., Molecular Cloning: A Laboratory Manual, 2nd edition (1989); Current Protocols In Molecular Biology (F. M. Ausubel, et al. eds., (1987)); the series Methods In Enzymology (Academic Press, Inc.); M. J. MacPherson, et al., eds. Pcr 2: A Practical Approach (1995); Harlow and Lane, eds, Antibodies: A Laboratory Manual (1988), and H. Jones, Methods In Molecular Biology vol. 49, “Plant Gene Transfer And Expression Protocols” (1995).

Immunoglobulin molecule or Antibody. A polypeptide or multimeric protein containing the immunologically active portions of an immunoglobulin heavy chain and immunoglobulin light chain covalently coupled together and capable of specifically combining with antigen. The immunoglobulins or antibody molecules are a large family of molecules that include several types of molecules such as IgD, IgG, IgA, secretory IgA (SIgA), IgM, and IgE.

Construct or Vector. An artificially assembled DNA segment to be transferred into a target plant tissue or cell. Typically, the construct will include the gene or genes of a particular interest, a marker gene and appropriate control sequences. The term “plasmid” refers to an autonomous, self-replicating extrachromosomal DNA molecule. In a preferred embodiment, the plasmid constructs of the present invention contain sequences coding for heavy and light chains of an antibody. Plasmid constructs containing suitable regulatory elements are also referred to as “expression cassettes.” In a preferred embodiment, a plasmid construct can also contain a screening or selectable marker, for example an antibiotic resistance gene.

Selectable marker. A gene that encodes a product that allows the growth of transgenic tissue on a selective medium. Non-limiting examples of selectable markers include genes encoding for antibiotic resistance, e.g., ampicillin, kanamycin, or the like. Other selectable markers will be known to those of skill in the art.

Transgenic plant. Genetically engineered plant or progeny of genetically engineered plants. The transgenic plant usually contains material from at least one unrelated organism, such as a virus, another plant or animal.

Chimeric ICAM-1 molecule: The fusion of any combination of the extracellular domains 1, 2, 3, 4 and 5 of ICAM-1 with at least a part of an immunoglobulin heavy chain protein, made by linking ICAM-1 sequence upstream of an immunoglobulin heavy chain gene sequence and expressing the encoded protein from the construct. In antibacterial embodiments, one or more receptor proteins effective to bind a bacterium or bacteria of interest or subcomponent thereof, such as a protein produced by the bacteria and required for the bacteria to exert its pathogenic effect, are used instead of, or in addition to, ICAM-1. Many such receptor proteins are known and can be implemented for use in appropriate aspects of the invention by those of ordinary skill in the art without exercising undue experimentation. An example is provided below utilizing one such protein that binds to a protein involved in the pathogenic mechanism caused by the bacterium that causes human anthrax. The same concept can be used to target other viruses besides rhinoviruses, e.g., by making use of appropriate host receptor proteins in immunoadhesin form.

Chimeric immunoglobulin heavy chain: An immunoglobulin derived heavy chain having at least a portion of its amino acid sequence derived from an immunoglobulin heavy chain of a different isotype or subtype or some other peptide, polypeptide or protein. Typically, a chimeric immunoglobulin heavy chain has its amino acid residue sequence derived from at least two different isotypes or subtypes of immunoglobulin heavy chain.

Dicotyledonous plants (dicots): Flowering plants whose embryos have two seed halves or cotyledons. Examples of dicots are: tobacco; tomato; the legumes including alfalfa; oaks; maples; roses; mints; squashes; daisies; walnuts; cacti; violets and buttercups.

Effective amount: An effective amount of an immunoadhesin of the present invention is sufficient to detectably inhibit viral or bacterial infection (as the case may be), cytotoxicity or replication; or to reduce the severity or length of infection.

Human rhinovirus (HRV): A nonenveloped RNA virus representing a subgroup of picomavirus, that is a major cause of the common cold in humans. Rhinoviruses are described in Rhinoviruses, Reoviruses, and Parvoviruses, pp. 1057-1059, Zinsser Microbiology, Joklik et al., eds. Appleton and Lange (1992).

Immunoadhesin: A complex containing a chimeric receptor protein molecule, and optionally containing secretory component, and J chain.

Immunoglobulin heavy chain: A polypeptide that contains at least a portion of the antigen binding domain of an immunoglobulin and at least a portion of a variable region of an immunoglobulin heavy chain or at least a portion of a constant region of an immunoglobulin heavy chain. Thus, the immunoglobulin derived heavy chain has significant regions of amino acid sequence homology with a member of the immunoglobulin gene superfamily. For example, the heavy chain in an Fab fragment is an immunoglobulin-derived heavy chain.

Immunoglobulin light chain: A polypeptide that contains at least a portion of the antigen binding domain of an immunoglobulin and at least a portion of the variable region or at least a portion of a constant region of an immunoglobulin light chain. Thus, the immunoglobulin-derived light chain has significant regions of amino acid homology with a member of the immunoglobulin gene superfamily.

Immunoglobulin molecule: A protein containing the immunologically-active portions of an immunoglobulin heavy chain and immunoglobulin light chain covalently coupled together and capable of specifically combining with antigen.

ICAM-1: Intercellular adhesion molecule-1. In humans, ICAM-1 functions as the receptor for human rhinovirus.

J chain: A polypeptide that is involved in the polymerization of immunoglobulins and transport of polymerized immunoglobulins through epithelial cells. See, The Immunoglobulin Helper: The J Chain in Immunoglobulin Genes, at pg. 345, Academic Press (1989). J chain is found in pentameric IgM and dimeric IgA and typically attached via disulphide bonds. J chain has been studied in both mouse and human.

Monocotyledonous plants (monocots): Flowering plants whose embryos have one cotyledon or seed leaf. Examples of monocots are: lilies; grasses; corn; grains, including oats, wheat and barley; orchids; irises; onions and palms.

Glycosylation: The modification of a protein by oligosaccharides. See, Marshall, Ann. Rev. Biochem., 41:673 (1972) and Marshall, Biochem. Soc. Symp., 40:17 (1974) for a general review of the polypeptide sequences that function as glycosylation signals. These signals are recognized in both mammalian and in plant cells.

Plant-specific glycosylation: The glycosylation pattern found on plant-expressed proteins, which is different from that found in proteins made in mammalian or insect cells. Proteins expressed in plants or plant cells have a different pattern of glycosylation than do proteins expressed in other types of cells, including mammalian cells and insect cells. Detailed studies characterizing plant-specific glycosylation and comparing it with glycosylation in other cell types have been performed by Cabanes-Macheteau et al., Glycobiology 9(4):365-372 (1999), Lerouge et al., Plant Molecular Biology 38:31-48 (1998) and Altmann, Glycoconjugate J. 14:643-646 (1997). Plant-specific glycosylation generates glycans that have xylose linked β(1,2) to mannose. Neither mammalian nor insect glycosylation generate xylose linked β(1,2) to mannose. Plants do not have a sialic acid linked to the terminus of the glycan, whereas mammalian cells do. In addition, plant-specific g lycosylation results in a fucose linked α(1,3) to the proximal GlcNAc, while glycosylation in mammalian cells results in a fucose linked α(1,6) to the proximal GlcNAc.

Secretory component (SC): A component of secretory immunoglobulins that helps to protect the immunoglobulin against inactivating agents thereby increasing the biological effectiveness of secretory immunoglobulin. The secretory component may be from any mammal or rodent including mouse or human.

sICAM: A naturally-occurring soluble truncated form of ICAM-1 lacking both the hydrophobic transmembrane domain and the carboxy-terminal cytoplasmic domain of ICAM.

The articles, patents and patent applications cited in this document are incorporated into this document as if set forth in full.

Although much of the discussion which follows is directed to ICAM-1 immunoadhesin aspects and embodiments, it will be clear to one of ordinary skill that other antiviral or antibacterial immunoadhesins can be similarly produced by incorporating receptor protein molecules other than ICAM-1. Indeed, examples 10-12 are directed to antibacterial embodiments in which the anthrax toxin receptor (ATR) is used instead of ICAM-1.

B. Immunoadhesins Containing Chimeric ICAM Molecules

The present invention provides novel methods for producing immunoadhesin molecules containing chimeric receptor proteins, e.g., ICAM-1 receptor proteins. ICAM-1 immunoadhesins, for example, contain chimeric ICAM-1 molecules made up of a rhinovirus receptor protein linked to a portion of an immunoglobulin heavy chain molecule in association with J chain and secretory component. The chimeric ICAM-1 molecules of such aspects of the present invention contain two molecules derived from different sources: a rhinovirus receptor protein portion and an immunoglobulin chain portion. The rhinovirus receptor protein is derived from the intercellular adhesion molecule 1 (ICAM-1). The nucleotide sequence for the human rhinovirus receptor ICAM-1 has been determined and characterized by Staunton, et al., Cell 52:925-933 (1988); Greve, et al. Cell 56:839-847 (1989); Greve, et al. J. Virology 65:6015-6023 (1991); Staunton, et al., Cell, 61:243-254 (1990) and described in Sequence ID No. 3 and GenBank accession no. M24283.

The ICAM-1 molecule is a membrane protein (SEQ ID NOS: 1 and 2) that has 5 extracellular domains, a hydrophobic transmembrane domain and a short cytoplasmic domain. These features have been described by Casasnovas, et al., Proc. Natl. Acad. Sci. U.S.A., 95:4134-4139 (1998) and Staunton, et al, Cell 52:925-933 (1988). Of particular use in appropriate aspects of the present invention are the domains of the ICAM-1 molecule that are responsible for the binding of human rhinoviruses which have been localized to the N-terminal domains 1 and 2 (Greve, et al., J. Virol., 65:6015-6023 1991, and Staunton, et al., Cell, 61:243-245 1990. Such aspects also contemplate(s) rhinovirus receptor protein portions which include any combination of extracellular domains 1, 2, 3, 4, and 5 of the ICAM-1 molecule. In particular preferred embodiments, the rhinovirus receptor protein portion includes domains 1 and 2 of the ICAM-1 molecule and in other preferred embodiments domains 1, 2, 3, 4 and 5 of the ICAM-1 molecule are present.

The boundaries of the 5 extracellular domains of ICAM-1 are well known in the art and described in Staunton, et al., Cell 52:925-933 (1988). The approximated domain boundaries are shown in Table 1 below [SEQ ID NO:2].

TABLE 1

| ICAM-1 Domains | Amino Acids |
|---|---|
| 1 | 1-88 |
| 2 | 89-105 |
| 3 | 106-284 |
| 4 | 285-385 |
| 5 | 386-453 |

As used in some aspects and embodiments of the present invention, the ICAM-1 domain 1 is from about residue 1 to about residue 88; domain 2 is from about residue 89 to about residue 105; domain 3 is from about residue 106 to about residue 284; domain 4 is from about residue 285 to about 385; and domain 5 is from about residue 386 to 453. One of skill in the art will understand that the exact boundaries of these domains may vary.

The chimeric ICAM-1 molecules preferably contain at least a portion of an IgM or IgA heavy chain which allows that immunoglobulin heavy chain to bind to immunoglobulin J chain and thereby binds to the secretory component. It is contemplated that the portion of the chimeric ICAM-1 molecule derived from the immunoglobulin heavy chain may be comprised of individual domains selected from the IgA heavy chain or the IgM heavy chain or from some other isotype of heavy chain. It is also contemplated that an immunoglobulin domain derived from an immunoglobulin heavy chain other than IgA or IgM or from an immunoglobulin light chain may be molecularly engineered to bind immunoglobulin J chain and thus may be used to produce immunoglobulins and immunoadhesins of the present invention.

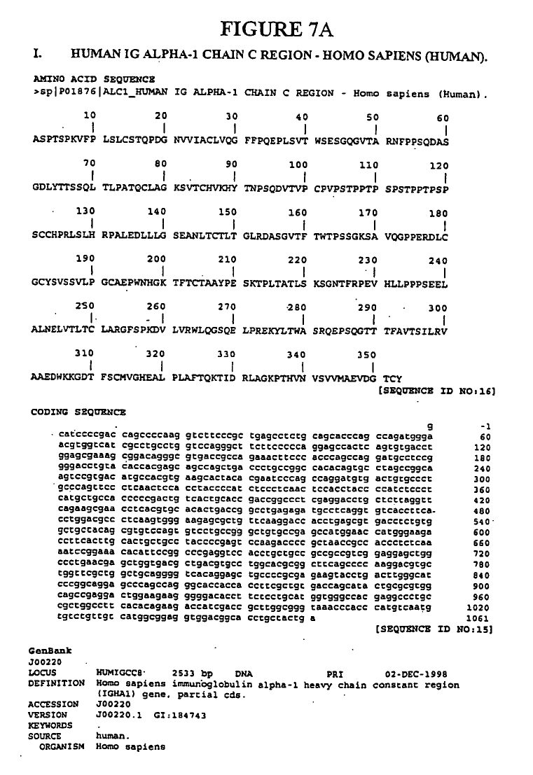

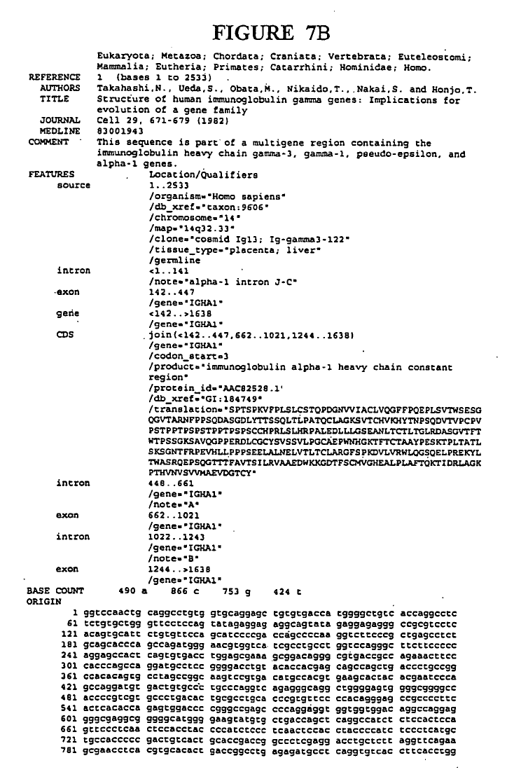

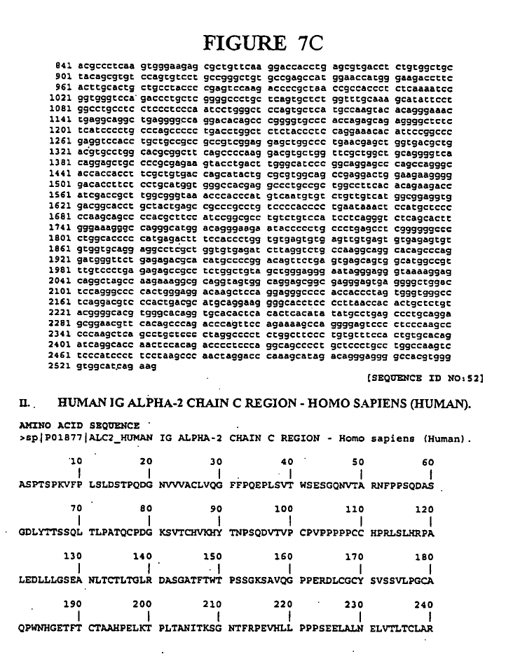

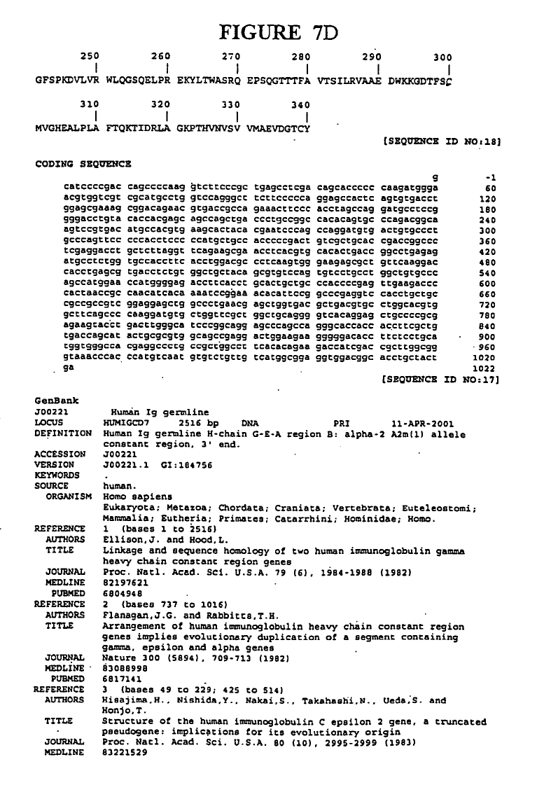

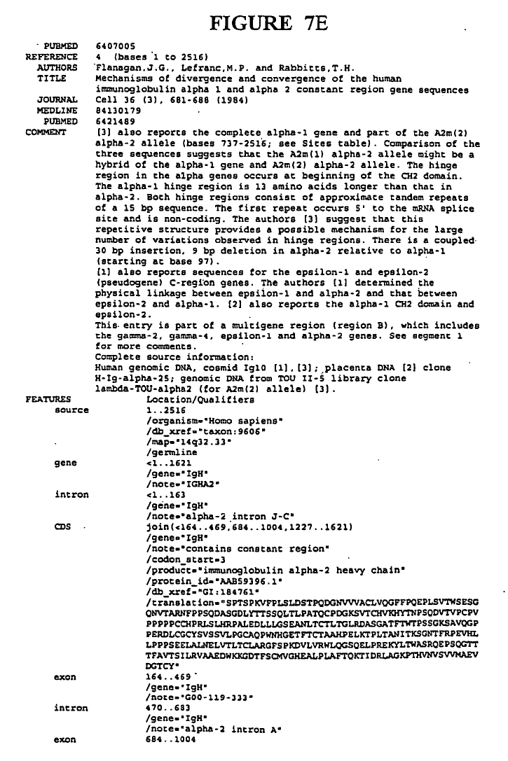

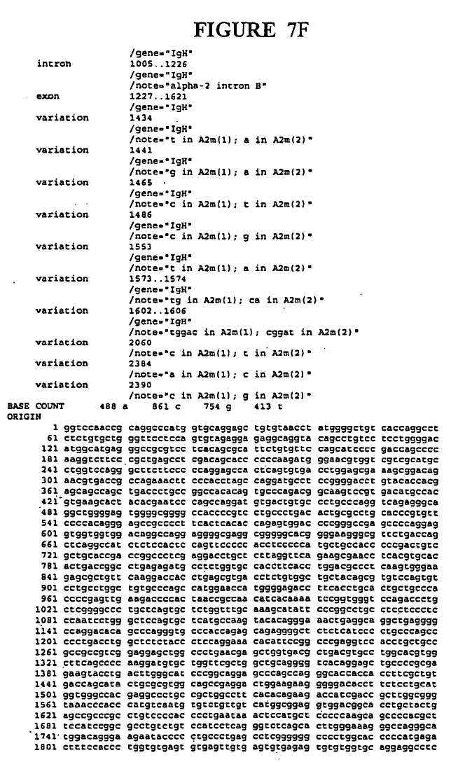

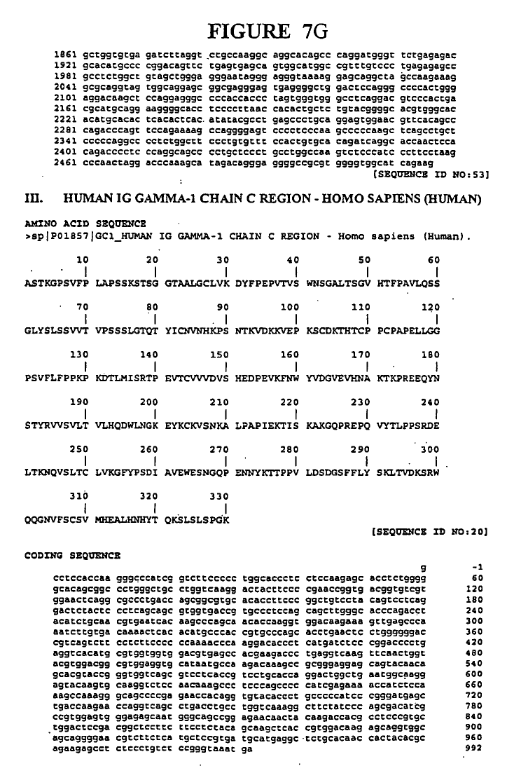

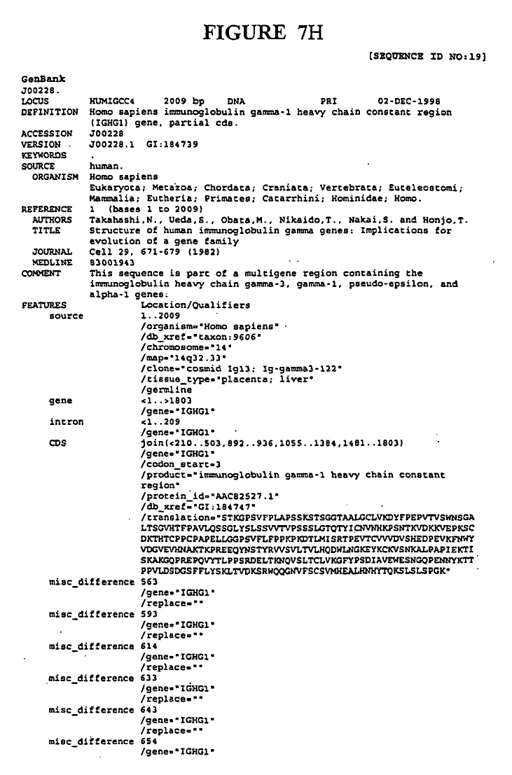

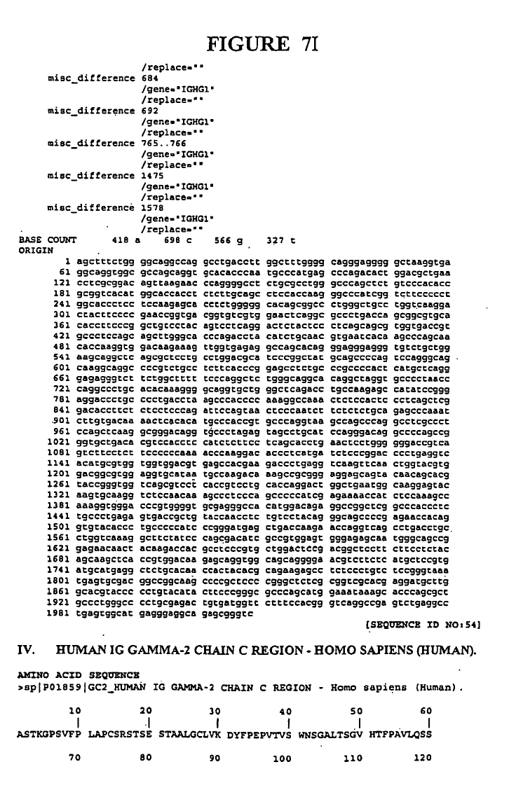

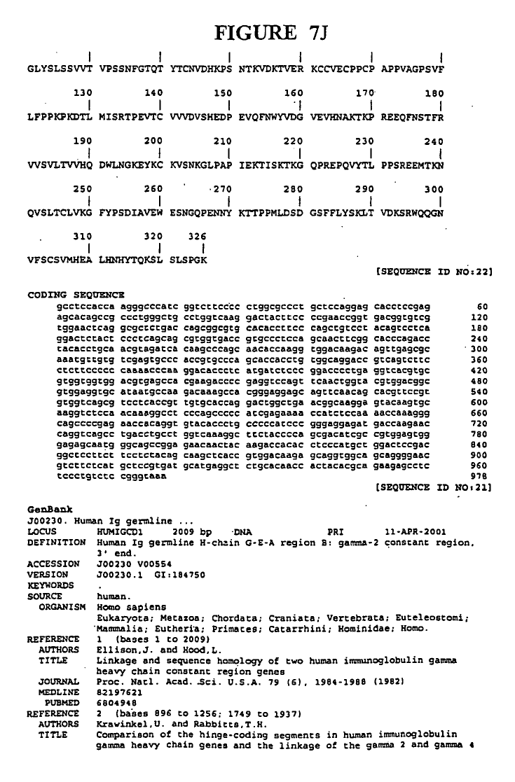

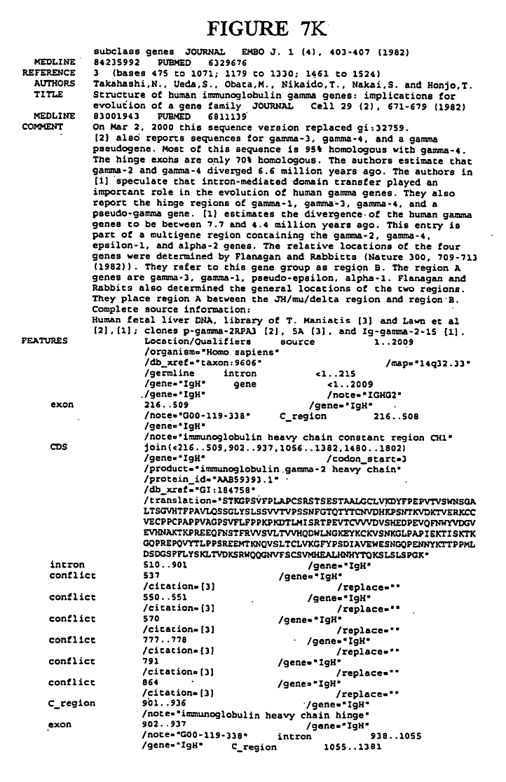

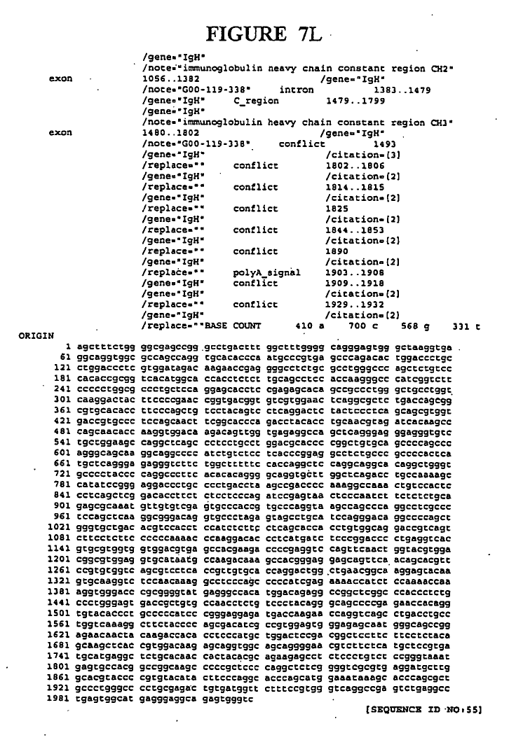

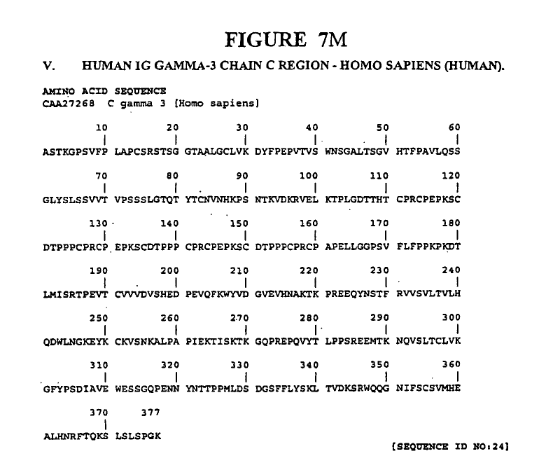

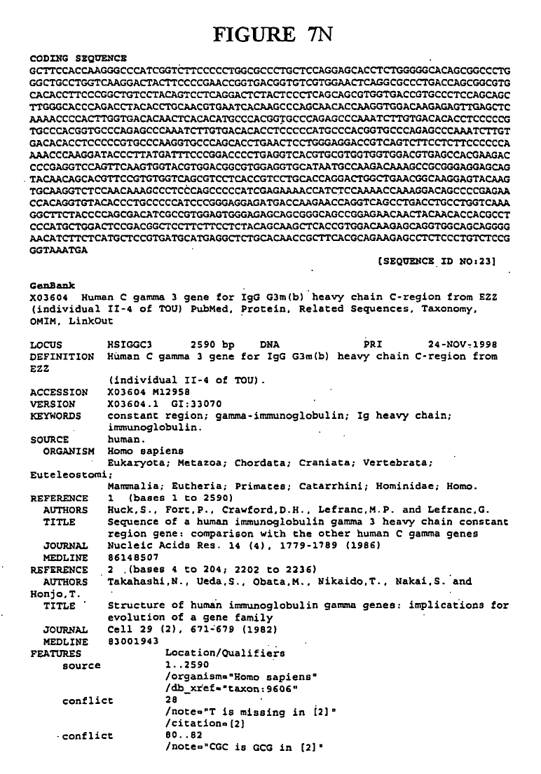

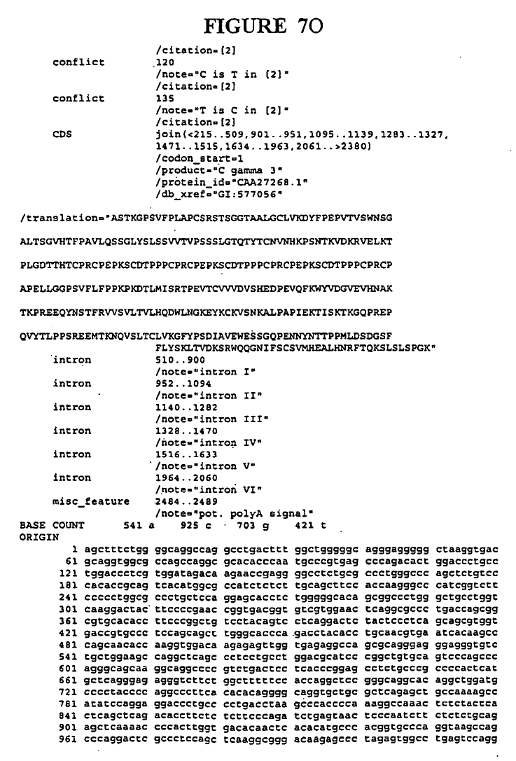

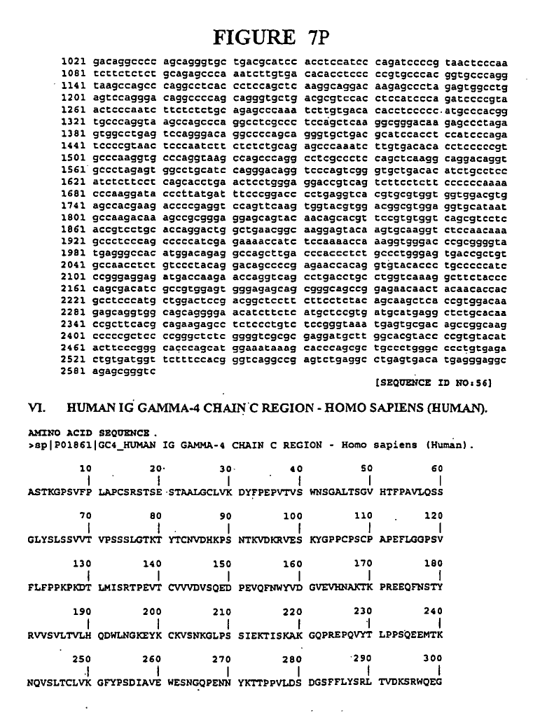

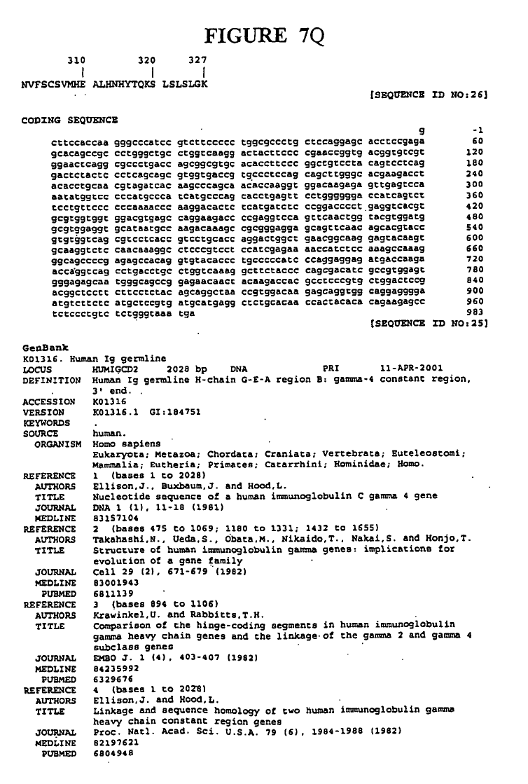

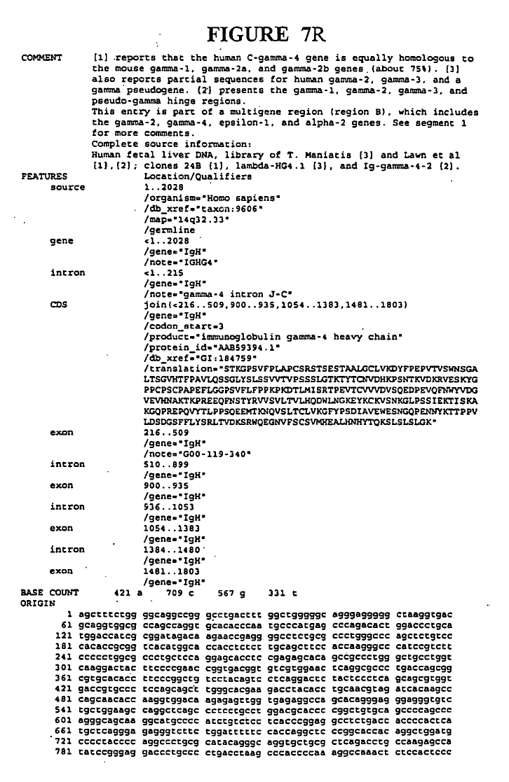

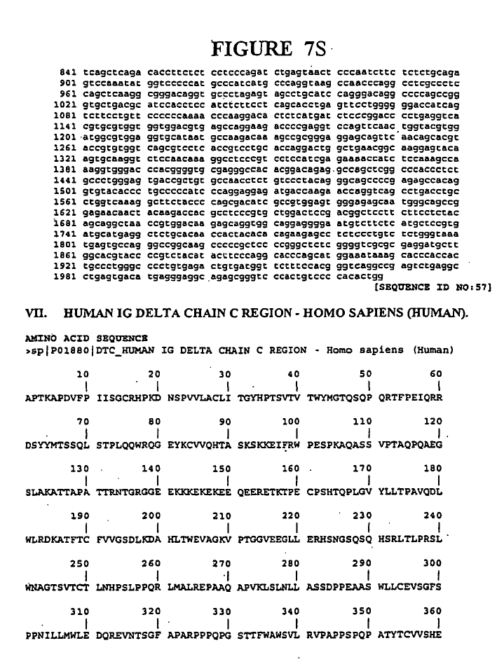

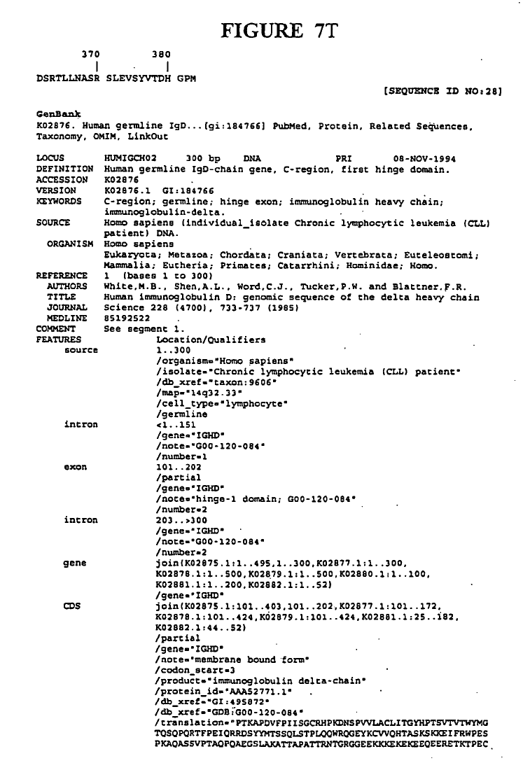

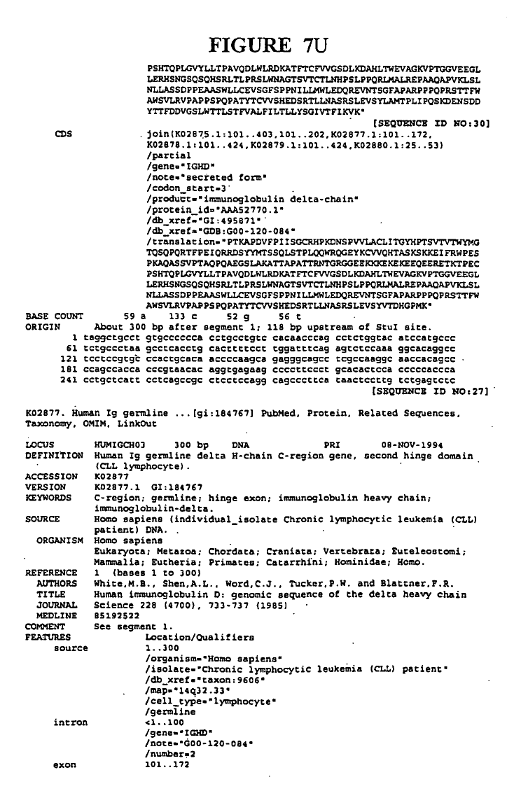

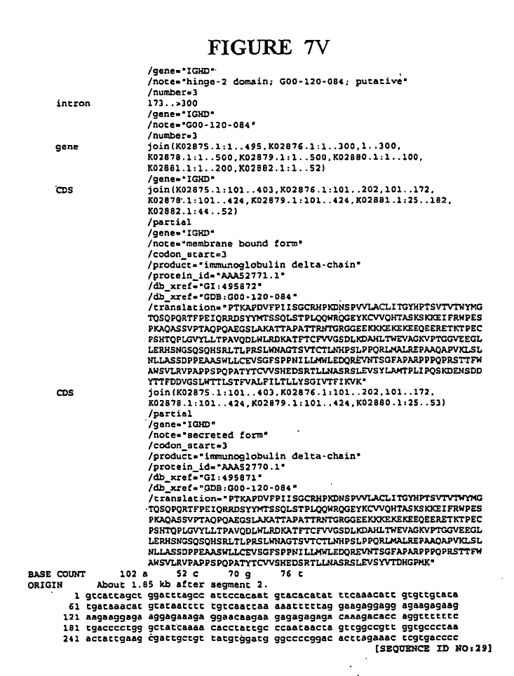

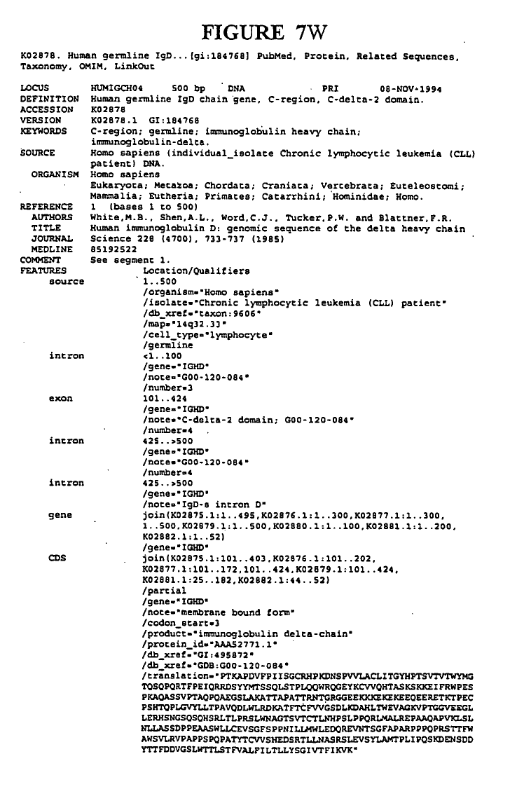

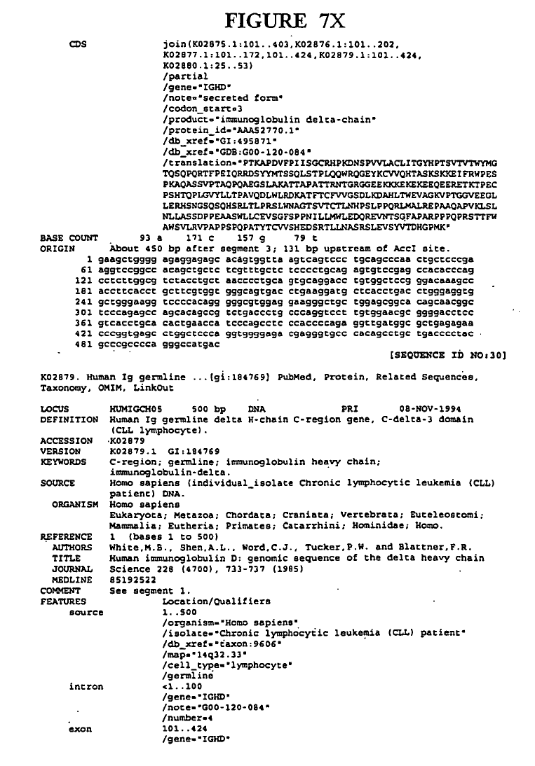

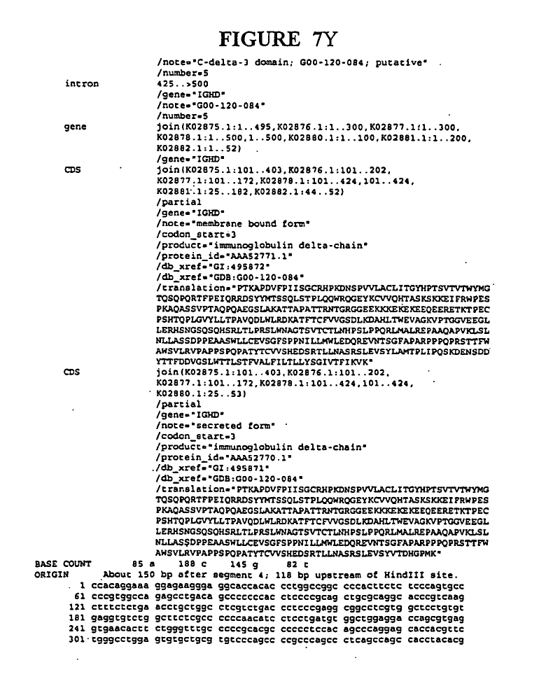

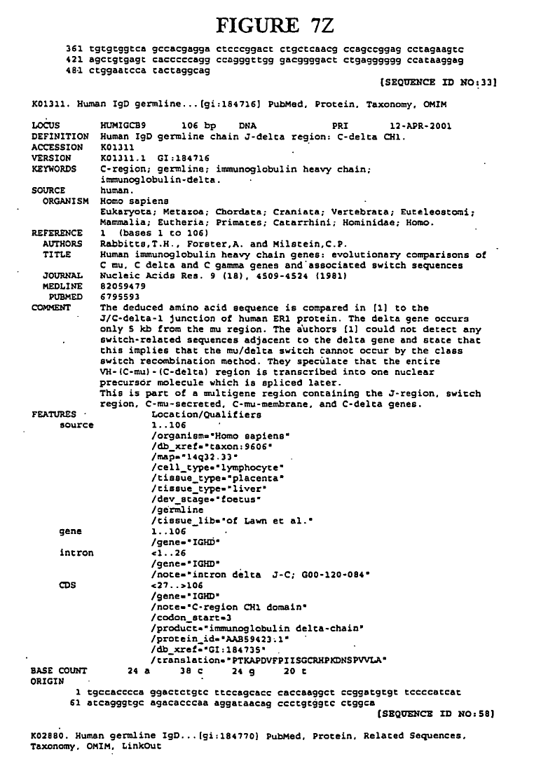

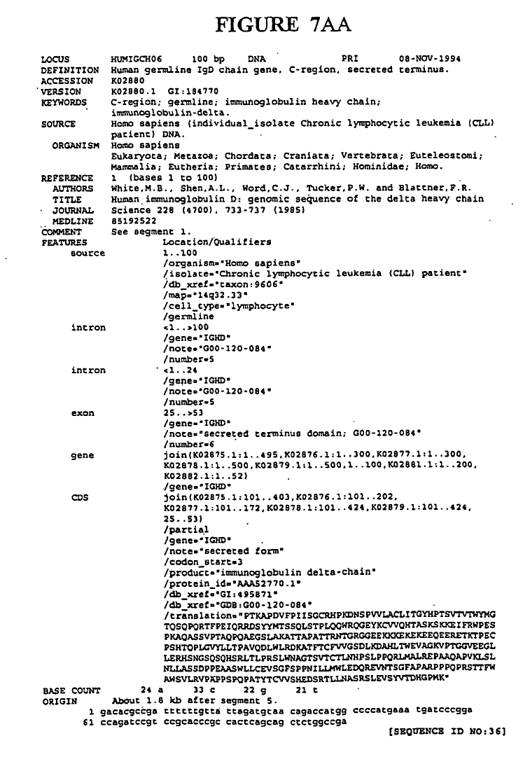

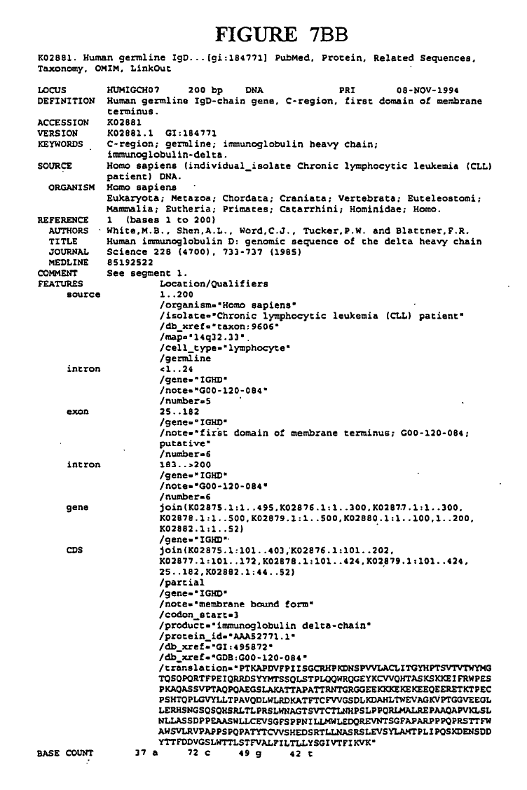

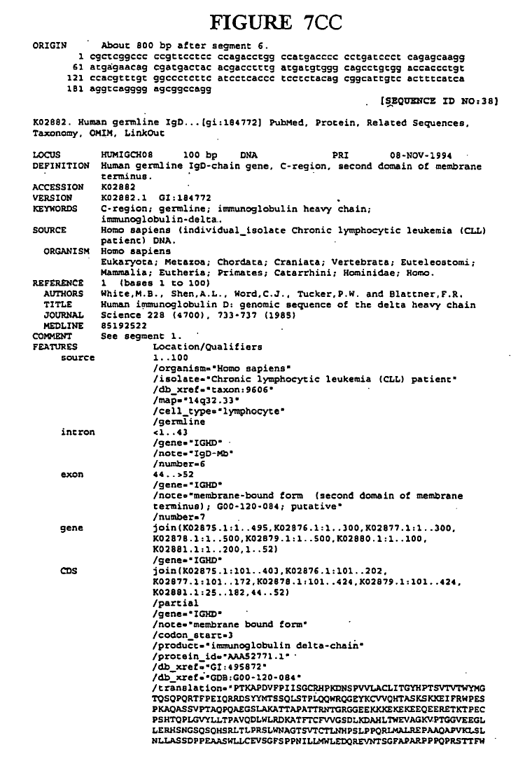

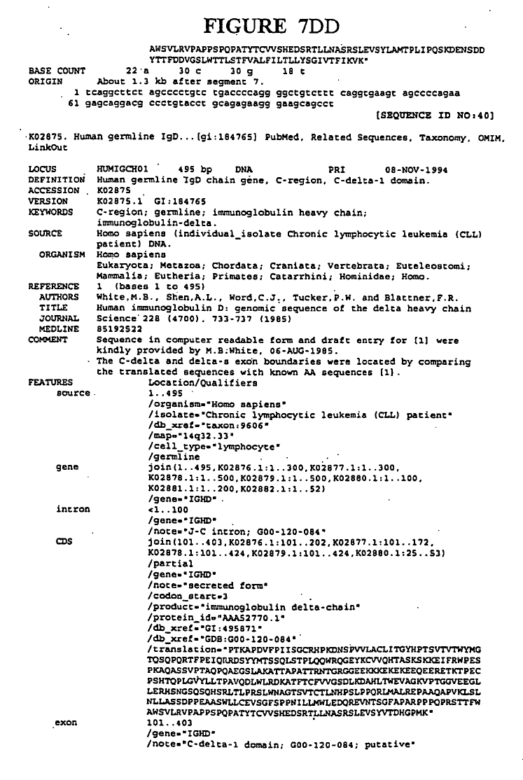

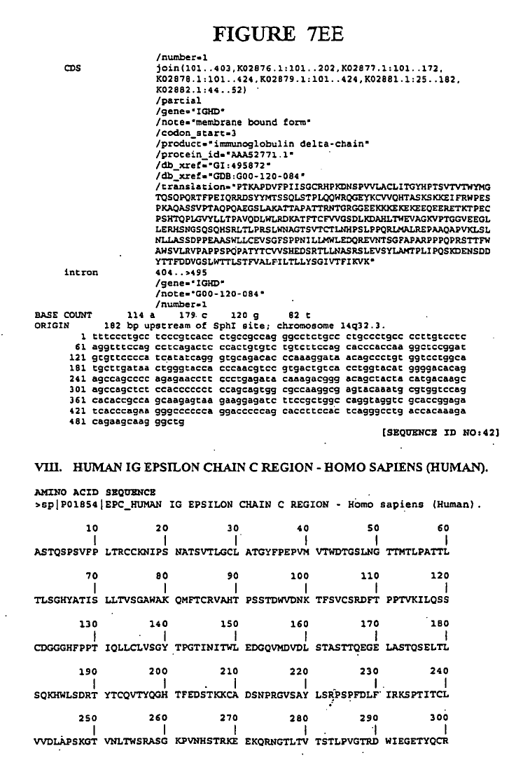

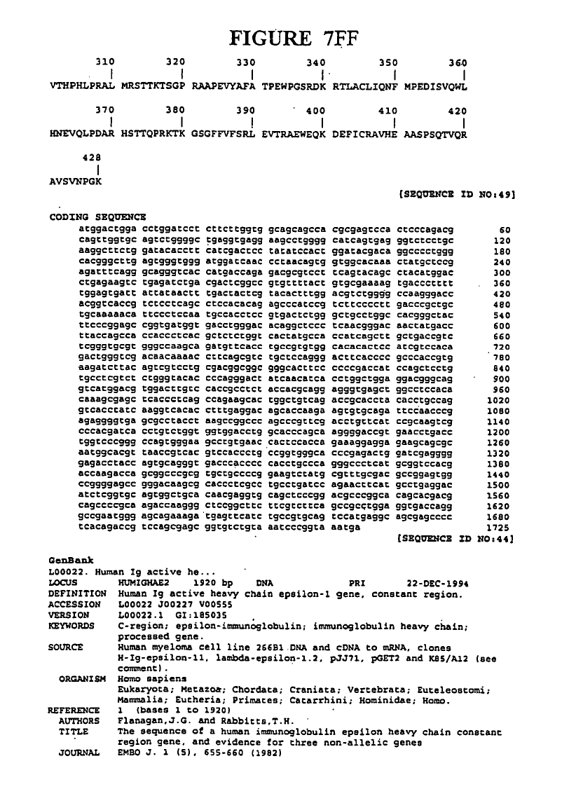

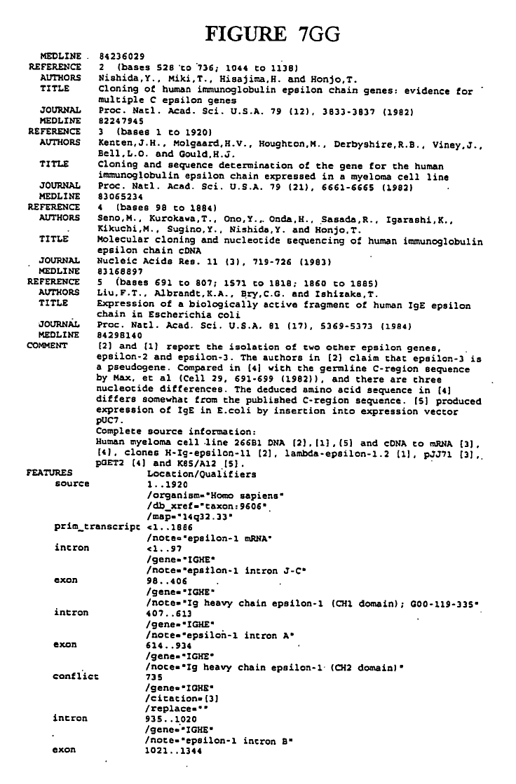

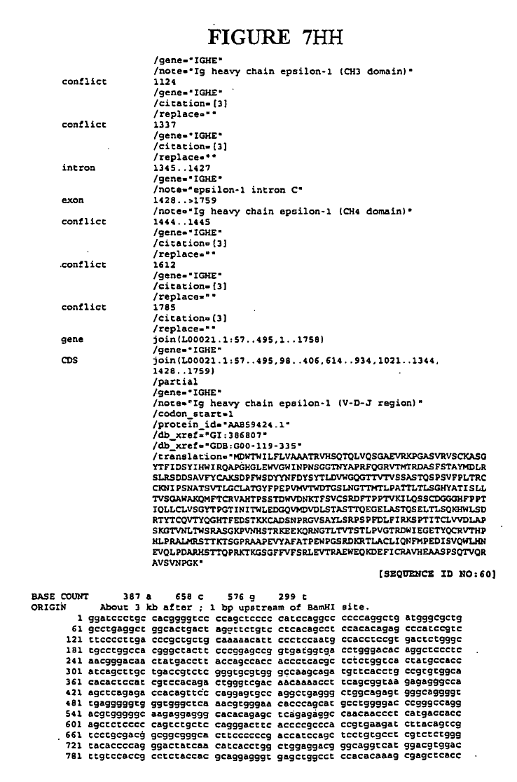

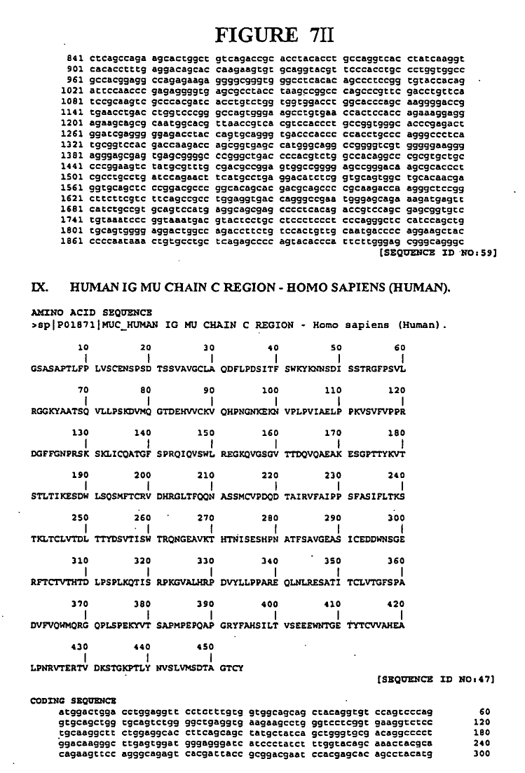

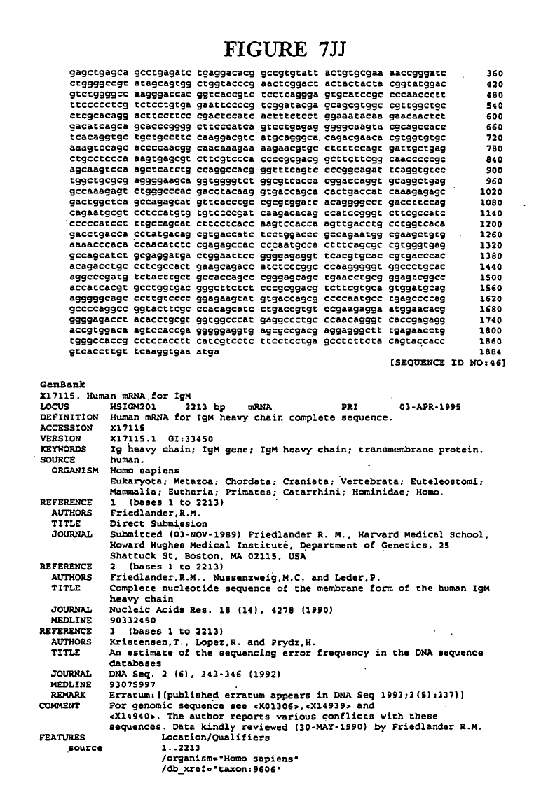

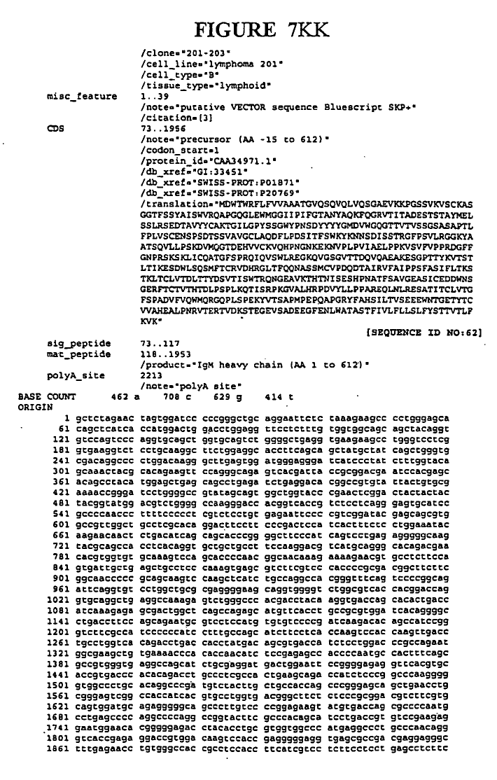

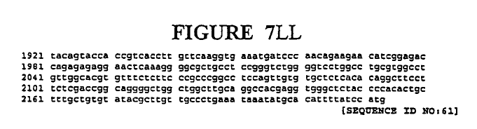

One skilled in the art will understand that immunoglobulins consist of domains which are approximately 100-110 amino acid residues. These various domains are well known in the art and have known boundaries. The removal of a single domain and its replacement with a domain of another antibody molecule is easily achieved with modern molecular biology. The domains are globular structures which are stabilized by intrachain disulfide bonds. This confers a discrete shape and makes the domains a self-contained unit that can be replaced or interchanged with other similarly shaped domains. The heavy chain constant region domains of the immunoglobulins confer various properties known as antibody effector functions on a particular molecule containing that domain. Example effector functions include complement fixation, placental transfer, binding to staphyloccal protein, binding to streptococcal protein G, binding to mononuclear cells, neutrophils or mast cells and basophils. The association of particular domains and particular immunoglobulin isotypes with these effector functions is well known and for example, described in Immunology, Roitt et al., Mosby St. Louis, Mo. (1993 3rd Ed.) One of skill in the art will be able to identify immunoglobulin heavy chain constant region sequences. For example, a number of immunoglobulin DNA and protein sequences are available through GenBank. Table 2 shows the GenBank Accession numbers of immunoglobulin heavy chain genes and the proteins encoded by the genes. The sequences listed in Table 2 are shown in FIG. 7.

TABLE 2

| GENBANK | | |
|---|---|---|
| ACCESSION | HUMAN IMMUNOGLOBULIN | SEQ |
| NO. | SEQUENCE NAME | ID NO. |
| J00220 | Ig_{α1} Heavy Chain Constant Region | 15, 52 |
| | Coding Sequence | |
| J00220 | Ig_{α1} Heavy Chain Constant Region Amino | 16 |
| | Acid Sequence | |
| J00221 | IgA₂ Heavy Chain Constant Region Coding | 17, 53 |
| | Sequence | |
| J00221 | IgA₂ Chain Constant Region Amino Acid | 18 |
| | Sequence | |
| J00228 | Ig_{γ1} Heavy Chain Constant Region Coding | 19, 54 |
| | Sequence | |
| J00228 | Ig_{γ1} Heavy Chain Constant Region Amino | 20 |
| | Acid Sequence | |
| J00230 | IgG₂ Heavy Chain Constant Region Coding | 21, 55 |
| V00554 | Sequence | |
| J00230 | IgG₂ Heavy Chain Constant Region Amino | 22 |
| V00554 | Acid Sequence | |
| X03604 | IgG₃ Heavy Chain Constant Region Coding | 23, 57 |
| M12958 | Sequence | |
| X03604 | IgG₃ Heavy Chain Constant Region Amino | 24 |
| M12958 | Acid Sequence | |
| K01316 | IgG₄ Heavy Chain Constant Region Coding | 25 |
| | Sequence | |
| K01316 | IgG₄ Heavy Chain Constant Region Amino | 26 |
| | Acid Sequence | |
| K02876 | IgD Heavy Chain Constant Region Coding | 27 |
| | Sequence | |
| K02876 | IgD Heavy Chain Constant Region Amino | 28, 30, 32 |
| | Acid Sequence | |
| K02877 | IgD Heavy Chain Constant Region Coding | 29 |
| | Sequence | |
| K02877 | IgD Heavy Chain Constant Region Amino | 28, 30, 32 |
| | Acid Sequence | |
| K02878 | Germline IgD Heavy Chain Coding | 31 |
| | Sequence | |
| K02878 | Germline IgD Heavy Chain Amino Acid | 28, 30, 32 |
| | Sequence | |
| K02879 | Germline IgD Heavy Chain C-δ-3 Domain | 33 |
| | Coding Sequence | |
| K02879 | Germline IgD Heavy Chain C-δ-3 Amino | 28, 30, 32 |
| | Acid Sequence | |
| K01311 | Germline IgD Heavy Chain J-δ Region: C-δ | 58 |
| | CH1 Coding Sequence | |
| K01311 | Germline IgD Heavy Chain J-δ Region: C-δ | 28, 30, 32 |
| | CH1 Amino Acid Sequence | |
| K02880 | Germline IgD Heavy Chain Gene, C-Region, | 36 |
| | Secreted Terminus Coding Sequence | |
| K02880 | Germline IgD Heavy Chain Gene, C-Region, | 28, 30, 32 |
| | Secreted Terminus Amino Acid Sequence | |
| K02881 | Germline IgD-Heavy Chain Gene, C-Region, | 38 |
| | First Domain of Membrane Terminus Coding | |
| | Sequence | |
| K02881 | Germline IgD-Heavy Chain Gene, C-Region, | 28, 30, 32 |
| | First Domain of Membrane Terminus Amino | |
| | Acid Sequence | |
| K02882 | Germline IgD Heavy Chain Coding | 40 |
| | Sequence | |
| K02882 | Germline IgD Heavy Chain Amino Acid | 28, 30, 32 |
| | Sequence | |
| K02875 | Germline IgD Heavy Chain Gene, C-Region, | 42 |
| | C-δ-1 Domain Coding Sequence | |
| K02875 | Germline IgD Heavy Chain Gene, C-Region, | 28, 30, 32 |
| | C-δ-1 Domain Amino Acid Sequence | |
| L00022 | IgE Heavy Chain Constant Region Coding | 59 |
| J00227 | Sequence | |
| V00555 | | |
| L00022 | IgE Heavy Chain Constant Region Amino | 60 |
| J00227 | Acid Sequence | |
| V00555 | | |
| X17115 | IgM Heavy Chain Complete Sequence | 61 |
| | Coding Sequence | |
| X17115 | IgM Heavy Chain Complete Sequence | 62 |
| | Amino Acid Sequence | |

The ICAM-1 immunoadhesins of the present invention may, in addition to the chimeric ICAM-1 molecule, contain immunoglobulin light chains, or immunoglobulin J chain bound to the immunoglobulin derived heavy chains. In preferred embodiments, the immunoadhesin of the present invention comprises two or four chimeric ICAM-1 molecules and an immunoglobulin J chain bound to at least one of the chimeric ICAM-1 molecules. The J chain is described and known in the art. See, for example, M. Koshland, The Immunoglobulin Helper: The J Chain, in Immunoglobulin Genes, Academic Press, London, pg. 345, (1989) and Matsuuchi et al., Proc. Natl. Acad. Sci. U.S.A., 83:456-460 (1986). The sequence of the immunoglobulin J chain is available on various databases in the United States.

The immunoadhesin may have a secretory component associated with the chimeric ICAM-1 molecule. This association may occur by hydrogen bonds, disulfide bonds, covalent bonds, ionic interactions or combinations of these various bonds. Typically, chimeric ICAM-1 molecules are held together by disulfide bonds between the molecules. The interaction of the chimeric ICAM-1 molecules may be non-covalent or disulfide bonding.

The present invention contemplates the use of secretory component from a number of different species, including human, rat, rabbit, bovine and the like. The nucleotide sequences for these molecules are well known in the art. For example, U.S. Pat. No. 6,046,037 contains many of the sequences and this patent is incorporated herein by reference. The immunoadhesins of the present invention containing the secretory component, the chimeric ICAM-1 molecule and J chain are typically bonded together by one of the following: hydrogen bonds, disulfide bonds, covalent bonds, ionic interactions or combinations of these bonds.

The present invention also contemplates immunoadhesins which comprise more than one receptor protein molecule, e.g., ICAM-1. ICAM-1 immunoadhesins, for example, may contain chimeric ICAM-1 molecules that are monomeric units and not disulfide bonded to other chimeric ICAM-1 molecules. In preferred embodiments, the immunoadhesin does contain chimeric ICAM-1 molecules that are in association with other chimeric ICAM-1 molecules to form dimers and other multivalent molecules. Typically the chimeric ICAM-1 molecule is present as a dimer because of the association of the immunoglobulin portion of the chimeric molecule. The immunoglobulin portion of the chimeric ICAM-1 molecule allows the association of two chimeric ICAM-1 molecules to form a dimeric molecule having two active binding portions made up of the rhinovirus receptor protein portion. In preferred embodiments, dimerization occurs via the disulfide bonding regions that normally occur between the immunoglobulin domains as part of a naturally-occurring immunoglobulin molecule and the native immunoglobulin protein. One of skill in the art will understand that these disulfide bonds that are normally present in the native immunoglobulin molecule can be modified, moved and removed while still maintaining the ability to form a dimer of the chimeric ICAM-1 molecules.

In other preferred embodiments, the immunoadhesin contains multimeric forms of the chimeric ICAM-1 molecule due to the association of J chain with the immunoglobulin portion of the chimeric ICAM molecule. The association of J chain with the dimer of two chimeric ICAM-1 molecules allows the formation of tetrameric forms of the immunoadhesin. In a preferred embodiment, the immunoglobulin portion of the chimeric ICAM-1 molecule is derived from the IgA molecule, and the addition of J chain allows the formation of a tetrameric complex containing four chimeric ICAM-1 molecules and four binding sites. In other preferred embodiments, the immunoglobulin heavy-chain portion of the chimeric molecule is derived from IgM and multivalent complexes containing ten or twelve molecules may be formed. In other preferred embodiments, in which the chimeric ICAM-1 molecule uses a chimeric immunoglobulin heavy-chain, the chimeric ICAM-1 molecule may form dimers or other higher order multivalent complexes through the domains from either IgA or IgM that are responsible for J chain binding. In other chimeric immunoglobulin molecules the portions of the immunoglobulin responsible for the disulfide bonding between the two immunoglobulin heavy-chains and/or the disulfide bonding between an immunoglobulin light-chain and heavy-chain may be placed in the chimeric immunoglobulin molecule to allow the formation of dimers or other high order multivalent complexes.

Various aspects and embodiments contemplate a chimeric ICAM-1 molecule in which the immunoglobulin domains comprising the heavy chain are derived from different isotypes of either heavy or light chain immunoglobulins. One skilled in the art will understand that using molecular techniques, these domains can be substituted for a similar domain and thus produce an immunoglobulin that is a hybrid between two different immunoglobulin molecules. These chimeric immunoglobulins allow immunoadhesins containing secretory component to be constructed that contain a variety of different and desirable properties that are conferred by different immunoglobulin domains.

Also contemplated are chimeric ICAM-1 molecules in which the portion of the chimeric molecule derived from immunoglobulin, heavy or light chain may contain less than an entire domain derived from a different immunoglobulin molecule. The same molecular techniques may be employed to produce such chimeric ICAM-1 molecules.

In preferred embodiments, the chimeric ICAM-1 molecules contain at least the CH1, CH2, CH3, domain of mouse or human IgA1, IgA2 or IgM. Other preferred embodiments of the present invention contain immunoglobulin domains that include at least the Cμ1, Cμ2, Cμ3, or Cμ4 domains of IgM.

Preferred chimeric ICAM-1 molecules contain domains from two different isotypes of human immunoglobulin. Preferred chimeric ICAM-1 molecules that include immunoglobulins that contain immunoglobulin domains including at least the CH1, CH2, or CH3 of human IgG, IgG1, IgG2, IgG3, IgG4, IgA1, IgA2, IgE, or IgD. Other preferred immunoglobulins for use as part of chimeric ICAM-1 molecules include immunoglobulins that contain domains from at least the CH1, CH2, CH3, or CH4 domain of IgM or IgE. The present invention also contemplates chimeric ICAM-1 molecules that contain immunoglobulin domains derived from at least two different isotypes of mammalian immunoglobulins. Generally, any of the mammalian immunoglobulins can be used in the preferred embodiments, such as the following isotypes: any isotype of IgG, any isotype of IgA, IgE, IgD or IgM. Chimeric ICAM-1 molecules derived from a species such as human, mouse or other mammals are contemplated. In preferred embodiments, the chimeric ICAM-1 molecule contains the portion of IgA or IgM responsible for the association of J chain with the IgA and IgM. Thus, by using a chimeric immunoglobulin in the chimeric ICAM-1 molecule, the J chain may associate with a chimeric immunoglobulin that is predominantly of an isotype that does not bind J chain or secretory component.

The present invention also contemplates chimeric molecules that contain immunoglobulin domains derived from two different isotypes of rodent or primate immunoglobulin. The isotypes of rodent or primate immunoglobulin are well known in the art. The chimeric molecules of the present invention may contain immunoglobulin derived heavy chains that include at least one of the following immunoglobulin domains: the CH1, CH2, or CH3 domains of a mouse IgG, IgG1, IgG2a, IgG2b, IgG3, IgA, IgE, or IgD; the CH1, CH2, CH3 or CH4 domain of mouse IgE or IgM; the CH1 domain of a human IgG, IgG1, IgG2, IgG3, IgG4, IgA1, IgA2, or IgD; the CH1, CH2, CH3, CH4 domain of human IgM or IgE; the CH1, CH2, or CH3 domain of an isotype of mammalian IgG, an isotype of IgA, IgE, or IgD; the CH1, CH2, CH3 or CH4 domain of a mammalian IgE or IgM; the CH1, CH2, or CH3 domain of an isotype of rodent IgG, IgA, IgE, or IgD; the CH1, CH2, CH3 or CH4 domain of a rodent IgE or IgM; the CH1, CH2, or CH3 domain of an isotype of animal IgG, an isotype of IgA, IgE, or IgD; and the CH1, CH2, CH3, or CH4 domain of an animal IgE or IgM. The present invention also contemplates the replacement or addition of protein domains derived from molecules that are members of the immunoglobulin superfamily into the chimeric molecules, e.g., chimeric ICAM-1. The molecules that belong to the immunoglobulin superfamily have amino acid residue sequence and nucleic acid sequence homology to immunoglobulins. The molecules that are part of the immunoglobulin superfamily can be identified by amino acid or nucleic acid sequence homology. See, for example, p. 361 of Immunoglobulin Genes, Academic Press (1989).

In preferred embodiments of the present invention, the immunoadhesin is expressed by methods that generate an immunoadhesin having plant-specific glycosylation. It is well-known in the art that glycosylation is a major modification of proteins in plant cells (Lerouge et al., Plant Molecular Biology 38:31-48, 1998). Glycosylation of proteins also occurs in other cell types, including mammalian and insect cells. The glycosylation process starts in the endoplasmic reticulum by the co-translational transfer of a precursor oligosaccharide to specific residues of the nascent polypeptide chain. Processing of this oligosaccharide into different types of glycans, which differ in the types of residues present and the nature of the linkages between the residues, occurs in the secretory pathway as the glycoprotein moves from the endoplasmic reticulum to its final destination. One of skill in the art will understand that at the end of their maturation, proteins expressed in plants or plant cells have a different pattern of glycosylation than do proteins expressed in other types of cells, including mammalian cells and insect cells. Detailed studies characterizing plant-specific glycosylation and comparing it with glycosylation in other cell types have been performed, for example, in studies described by Cabanes-Macheteau et al., Glycobiology 9(4):365-372 (1999), and Altmann, Glycoconjugate J. 14:643-646 (1997). These groups and others have shown that plant-specific glycosylation generates glycans that have xylose linked β(1,2) to mannose, but xylose is not linked β(1,2) to mannose as a result of glycosylation in mammalian and insect cells. Plant-specific glycosylation results in a fucose linked α(1,3) to the proximal GlcNAc, while glycosylation in mammalian cells results in a fucose linked α(1,6) to the proximal GlcNAc. Furthermore, plant-specific glycosylation does not result in the addition of a sialic acid to the terminus of the protein glycan, whereas in glycosylation in mammalian cells, sialic acid is added.

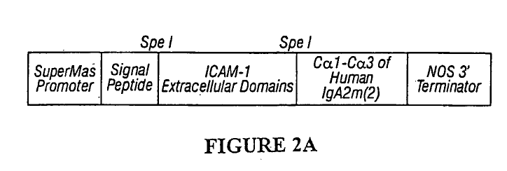

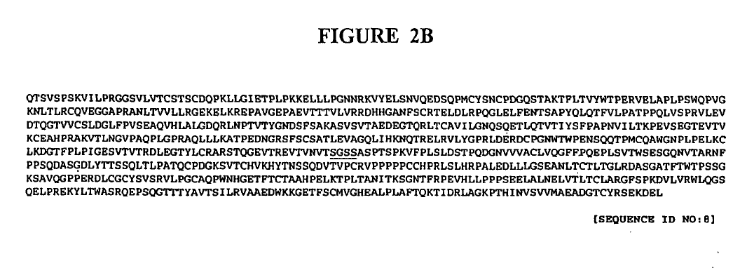

The immunoadhesin of the present invention that is glycosylated in a plant-specific manner can contain a chimeric molecule, e.g., chimeric ICAM-1, that includes any combination of extracellular domains, e.g., domains 1, 2, 3, 4, and 5 of the ICAM-1 molecule. FIG. 2B shows the amino acid sequence of the chimeric ICAM-1/IgA2 molecule (SEQ ID NO: 8) of the present invention, that contains all five domains of ICAM-1. The bolded N's represent asparagine residues to which oligosaccharide moieties are linked during glycosylation in plant cells, as well as mammalian and insect cells. One of skill in the art will know that the glycosylation sites are the tripeptide Asn-X-Ser/Thr where X can be any amino acid except proline and aspartic acid (Kornfeld and Kornfeld, Annu Rev Biochem 54:631-664, 1985). It will therefore be known to one of skill in the art that which amino acids of the protein having plant-specific glycosylation would depend on which domains of ICAM-1 are present. Because the sequence and domain boundaries of ICAM-1 are known (see Staunton et. al., Cell 52:925-933, 1988), it would be evident to one of skill in the art how to determine the plant-specific glycosylation sites on any potential combination of any of the five ICAM-1 domains.

In other preferred ICAM-1 aspects and embodiments of the present invention, the immunoadhesin having plant-specific glycosylation and containing a chimeric ICAM-1 molecule having any combination of ICAM-1 extracellular domains 1, 2, 3, 4 and 5 further comprises a J chain and secretory component associated with said chimeric ICAM-1 molecule. As was true with respect to the chimeric ICAM-1 molecule, one of skill in the art will be able to identify the sites for plant-specific glycosylation in the J chain and secretory component sequences. The same principle applies for immunoahesins of the invention that contain chimeric receptor proteins other than chimeric ICAM-1.

The present invention contemplates immunoadhesins having plant-specific glycosylation, that contain a chimeric molecule, e.g., a chimeric ICAM-1 molecule, in which the immunoglobulin heavy chain is selected from the group of IgA (SEQ ID NOS:15-18 and 52-53), IgA1 (SEQ ID NOS:15-16 and 52), IgA2 (SEQ ID NO:17 and 53), IgG1 (SEQ ID NOS:19-20 and 54), IgG2 (SEQ ID NOS:21-22 and 55), IgG3 (SEQ ID NOS:23-24 and 56), IgG4 (SEQ ID NOS:25-26 and 57), IgM (SEQ ID NOS:46-47 and 61-62), IgD (SEQ ID NOS:27-33, 35-36, 38,40, and 42), IgE (SEQ ID NOS:44-45 and 59-60), and a chimeric immunoglobulin heavy chain. One of skill in the art will know that which of these immunoglobulin heavy chain sequences, or which combination of immunoglobulin heavy chain sequences are combined in a chimeric immunoglobulin heavy chain, will have an effect on the number and location of glycosylation sites in the chimeric molecule of the immunoadhesin. As was true with respect to the chimeric molecule, one of skill in the art will be able to identify the sites for plant-specific glycosylation in the immunoglobulin heavy chain sequences, including the various chimeric immunoglobulin heavy chain sequences that can be constructed.

Also provided herein are immunoadhesin functional derivatives. By “functional derivative” is meant a “chemical derivative,” “fragment,” or “variant,” of the polypeptide or nucleic acid of the invention which retains at least a portion of the function of the protein, for example reactivity with an antibody specific for the protein, enzymatic activity or binding activity, which permits its utility in accordance with the present invention. It is well known in the art that due to the degeneracy of the genetic code numerous different nucleic acid sequences can code for the same amino acid sequence. It is also well known in the art that conservative changes in amino acid can be made to arrive at a protein or polypeptide that retains the functionality of the original. In both cases, all permutations are intended to be covered by this disclosure.

The derivatives may also be engineered according to routine methods to include an affinity purification tag such that large quantities and/or relatively pure or isolated quantities of immunoadhesin may be produced. Many different versions of tag exist that can be incorporated into one or more components of the immunoadhesin, preferably not destroying the desired binding activity of the immunoadhesin in the absence of tag. Such tags can be engineered as expressible encoded nucleic acid sequence fused with nucleic acid sequences encoding the immunoadhesins of the invention. The tags may further be engineered to be removable, e.g., with a commercially available enzyme.

Further, it is possible to delete codons or to substitute one or more codons with codons other than degenerate codons to produce a structurally modified polypeptide, but one which has substantially the same utility activity as the polypeptide produced by the unmodified nucleic acid molecule. As recognized in the art, the two polypeptides can be functionally equivalent, as are the two nucleic acid molecules that give rise to their production, even though the differences between the nucleic acid molecules are not related to the degeneracy of the genetic code.

Manipulations of this sort, and post-production chemical derivatization may be implemented, e.g., to improve stability, solubility, absorption, biological or therapeutic effect, and/or biological half-life. Moieties capable of mediating such effects are disclosed, for example, in Remington's Pharmaceutical Sciences, 18th ed., Mack Publishing Co., Easton, Pa. (1990). A functional derivative intended to be within the scope of the present invention is a “variant” polypeptide which either lacks one or more amino acids or contains additional or substituted amino acids relative to the native polypeptide. The variant may be derived from a naturally occurring complex component by appropriately modifying the protein DNA coding sequence to add, remove, and/or to modify codons for one or more amino acids at one or more sites of the C-terminus, N-terminus, and/or within the native sequence. It is understood that such variants having added, substituted and/or additional amino acids retain one or more characterizing portions of the native protein, as described above.

A functional derivative of a protein with deleted, inserted and/or substituted amino acid residues may be prepared using standard techniques well-known to those of ordinary skill in the art. For example, the modified components of the functional derivatives may be produced using site-directed mutagenesis techniques (as exemplified by Adelman et. al., 1983, DNA 2:183) wherein nucleotides in the DNA coding sequence are modified such that a modified coding sequence is produced, and thereafter expressing this recombinant DNA in a prokaryotic or eukaryotic host cell, using techniques such as those described above. Alternatively, proteins with amino acid deletions, insertions and/or substitutions may be conveniently prepared by direct chemical synthesis, using methods well-known in the art. The functional derivatives of the proteins typically exhibit the same qualitative biological activity as the native proteins.

In addition, the immunoadhesins of the invention may be not just modified receptor protein/Ig immunoadhesins, but may also embrace other native receptor protein family members, isotypes, and/or other homologous amino acid sequences, e.g., human, primate, rodent, canine, feline, bovine, avian, etc. Furthermore, the Ig type used in the immunoadhesins can vary, e.g., may assume a different Ig family member identity, within or without a given species. ICAMs and Igs, for example, are diverse and have well-known sequences that one of ordinary skill can exploit to create different immunoadhesins having more or less different utility in a given organism to undergo treatment. An illustrative, nonexhaustive list of examples of molecules having ICAM-1 homology that can be used to create other immunoadhesins include those in the following table.

TABLE 3

| ACCES- | | |
|---|---|---|
| SION NO. | ICAM NAME | SPECIES |
| NP 000192 | [SEQ ID NO:63] | Homo sapiens |
| | Intercellular Adhesion Molecule-1 | |
| | (CD54) | |
| AAH03097 | [SEQ ID NO:64] | Homo sapiens |
| | Intercellular Adhesion Molecule | |
| | ICAM-2 | |
| NP 002153 | [SEQ ID NO:65] | Homo sapiens |
| | Intercellular Adhesion Molecule 3 | |
| | Precursor | |
| BAB20325 | [SEQ ID NO:66] | Homo sapiens |
| | TCAM-1 | |
| NP 003250 | [SEQ ID NO:67] | Homo sapiens |
| | Intercellular Adhesion Molecule 5 | |
| | (Telencephalin) | |
| NM 007164 | [SEQ ID NO:68] | Homo sapiens |
| | Mucosal Vascular Address in Cell | |
| | Adhesion Molecule (MADCAM1) | |
| NM 001078 | [SEQ ID NO:69] | Homo sapiens |
| | Vascular Cell Adhesion Molecule 1 | |
| | (VCAM 1) | |
| AAA37875 | [SEQ ID NO:70] | Mus musculus |
| | MALA-2 | |
| AAA37876 | [SEQ ID NO:71] | Mus musculus |
| | Intercellular Adhesion Molecule-1 | |
| | Precursor | |
| AAG30280 | [SEQ ID NO:72] | Cricetulus griseus |
| | Intercellular Adhesion Molecule 1 | |
| AAB39264 | [SEQ ID NO:73] | Bos taurus |
| | Intercellular Adhesion Molecule-3 | |
| AAF80287 | [SEQ ID NO:74] | Sus scrofa |
| | Intercellular Adhesion Molecule-1 | |
| | Precursor | |
| AAA18478 | [SEQ ID NO:75] | Oryctolagus cimiculus |
| | Telecephalin | |
| NP 032345 | [SEQ ID NO:76] | Mus musculus |
| | Intercellular Adhesion Molecule 5, | |
| | telencephalin | |
| BAB41106 | [SEQ ID NO:77] | Mus musculus |
| | Cell adhesion molecule TCAM-1 | |
| NP 067705 | [SEQ ID NO:78] | Rattus norvegicus |
| | Testicular Cell Adhesion Molecule 1 | |
| AAG35584 | [SEQ ID NO:79] | Mus musculus |
| | Nectin-Like Protein 1 | |
| AAC18956 | [SEQ ID NO:80] | Homo sapiens |
| | CD22 Protein | |
| AAA35415 | [SEQ ID NO:81] | Pan troglodytes |
| | Intercellular Adhesion Molecule 1 | |
| AAA83206 | [SEQ ID NO:82] | Mus musculus |
| | 89 kDa Protein | |
| AAA92551 | [SEQ ID NO:83] | Canis familiaris |
| | Intercellular Adhesion Molecule-1 | |
| AAB06749 | [SEQ ID NO:84] | Bos taurus |
| | Intercellular Adhesion Molecule-1 | |
| AAD13617 | [SEQ ID NO:85] | Ovis aries |
| | Intercellular Adhesion Molecule-1 | |
| | Precursor | |
| NP 037099 | [SEQ ID NO:86] | Rattus norvegicus |
| | Intercellular Adhesion Molecule-1 | |
| AAE22202 | [SEQ ID NO:87] | Rattus norvegicus |
| | ICAM-4 | |
| AAA60392 | [SEQ ID NO:88] | Homo sapiens |
| | cell surface glycoprotein | |
| AAF91086 | [SEQ ID NO:89] | Rattus norvegicus |
| | Nephrin | |
| AAF91087 | [SEQ ID NO:90] | Mus musculus |

Nephrin

Likewise, numerous heavy chain constant regions of different Ig molecules, both in humans and other species, are known that can be substituted in for those specific Ig regions of the chimeras described herein.

C. Vectors, Cells and Plants Containing Immunoadhesins

The present invention also contemplates expression and cloning vectors, cells and plants containing the immunoadhesins of the present invention. Technology for isolating the genes encoding the various portions of the immunoadhesions are well-known to one of skill in the art and can be applied to insert the various required genes into expression vectors and cloning vectors such as those vectors can be introduced into cells and into transgenic plants.

The present invention contemplates a method of assembling an immunoadhesin comprising the steps of: introducing into an organism a DNA segment encoding a chimeric receptor protein molecule (e.g., an ICAM-1 molecule), immunoglobulin J chain, and introducing into the same organism a DNA encoding a secretory component. The preferred secretory component contains at least a segment of the amino acid residues 1 to residue about 606 of the human polyimmunoglobulin receptor (pIgR) amino acid residue sequence or analogous amino acid residues from other species (Mostov, Ann Dev. Immu. 12:63-84 1994).

The present invention contemplates eukaryotic cells, including plant cells, containing immunoadhesins of the present invention. The present invention also contemplates plant cells that contain nucleotide sequences encoding the various components of the immunoadhesin of the present invention. One skilled in the art will understand that the nucleotide sequences that encode the secretory component protection protein and the chimeric receptor protein molecule and J chain will typically be operably linked to a promoter and present as part of an expression vector or cassette. Typically, if the eukaryotic cell used is a plant cell then the promoter used will be a promoter that is able to operate in a plant cell. After the chimeric receptor protein genes, secretory component genes and J chain genes are isolated, they are typically operatively linked to a transcriptional promoter in an expression vector. The present invention also contemplates expression vectors containing a nucleotide sequence encoding a chimeric receptor protein molecule which has been operatively linked to a regulatory sequence for expression. These expression vectors place the nucleotide sequence to be expressed in a particular cell 3′ of a promoter sequence which causes the nucleotide sequence to be transcribed and expressed. The expression vector may also contain various enhancer sequences which improve the efficiency of this transcription. In addition, such sequences as terminators, polyadenylation (poly A) sites and other 3′ end processing signals may be included to enhance the amount of nucleotide sequence transcribed within a particular cell.

Expression of the components in the organism of choice can be derived from an independently replicating plasmid, or from a permanent component of the chromosome, or from any piece of DNA which may transiently give rise to transcripts encoding the components. Organisms suitable for transformation can be either prokaryotic or eukaryotic. Introduction of the components of the complex can be by direct DNA transformation, by biolistic delivery into the organism, or mediated by another organism as for example by the action of recombinant Agrobacterium on plant cells. Expression of proteins in transgenic organisms usually requires co-introduction of an appropriate promoter element and polyadenylation signal. In one embodiment of the invention, the promoter element potentially results in the constitutive expression of the components in all of the cells of a plant. Constitutive expression occurring in most or all of the cells will ensure that precursors can occupy the same cellular endomembrane system as might be required for assembly to occur.

Expression vectors compatible with the host cells, preferably those compatible with plant cells are used to express the genes of the present invention. Typical expression vectors useful for expression of genes in plants are well known in the art and include vectors derived from the tumor-inducing (Ti) plasmid of Agrobacterium tumefaciens described by Rogers et al., Meth. in Enzymol., 153:253-277 (1987). However, several other expression vector systems are known to function in plants. See for example, Verma et al., PCT Publication No. WO87/00551; and Cocking and Davey, Science, 236:1259-1262 (1987).

The expression vectors described above contain expression control elements including the promoter. The genes to be expressed are operatively linked to the expression vector to allow the promoter sequence to direct RNA polymerase binding and synthesis of the desired polypeptide coding gene. Useful in expressing the genes are promoters which are inducible, viral, synthetic, constitutive, and regulated. The choice of which expression vector is used and ultimately to which promoter a nucleotide sequence encoding part of the immunoadhesin of the present invention is operatively linked depends directly, as is well known in the art, on the functional properties desired, e.g. the location and timing of protein expression, and the host cell to be transformed, these being limitations inherent in the art of constructing recombinant DNA molecules. However, an expression vector useful in practicing the present invention is at least capable of directing the replication, and preferably also the expression of the polypeptide coding gene included in the DNA segment to which it is operatively linked.

In preferred embodiments, the expression vector used to express the genes includes a selection marker that is effective in a plant cell, preferably a drug resistance selection marker. A preferred drug resistance marker is the gene whose expression results in kanamycin resistance, i.e., the chimeric gene containing the nopaline synthase promoter, Tn5 neomycin phosphotransferase II and nopaline synthase 3′ nontranslated region described by Rogers et al., in Methods For Plant Molecular Biology, a Weissbach and H. Weissbach, eds., Academic Press Inc., San Diego, Calif. (1988). A useful plant expression vector is commercially available from Pharmacia, Piscataway, N.J. Expression vectors and promoters for expressing foreign proteins in plants have been described in U.S. Pat. Nos. 5,188,642; 5,349,124; 5,352,605, and 5,034,322 which are hereby incorporated by reference.

A variety of methods have been developed to operatively link DNA to vectors via complementary cohesive termini. For instance, complementary homopolymer tracks can be added to the DNA segment to be inserted into the vector DNA. The vector and DNA segment are then joined by hydrogen bonding between the complementary homopolymeric tails to form recombinant DNA molecules. Alternatively, synthetic linkers containing one or more restriction endonuclease sites can be used to join the DNA segment to the expression vector. The synthetic linkers are attached to blunt-ended DNA segments by incubating the blunt-ended DNA segments with a large excess of synthetic linker molecules in the presence of an enzyme that is able to catalyze the ligation of blunt-ended DNA molecules, such as bacteriophase T4 DNA ligase. Thus, the products of the reaction are DNA segments carrying synthetic linker sequences at their ends. These DNA segments are then cleaved with the appropriate restriction endonuclease and ligated into an expression vector that has been cleaved with an enzyme that produces termini compatible with those of the synthetic linker. Synthetic linkers containing a variety of restriction endonuclease sites are commercially available from a number of sources including New England BioLabs, Beverly, Mass.

The nucleotide sequences encoding the secretory component, J chain, and the chimeric receptor protein molecules, e.g., ICAM-1, of the present invention are introduced into the same plant cell either directly or by introducing each of the components into a plant cell and regenerating a plant and cross-hybridizing the various components to produce the final plant cell containing all the required components.

Any method may be used to introduce the nucleotide sequences encoding the components of the immunoadhesins of the present invention into a eukaryotic cell. For example, methods for introducing genes into plants include Agrobacterium-mediated plant transformation, protoplast transformation, gene transfer into pollen, injection into reproductive organs and injection into immature embryos. Each of these methods has distinct advantages and disadvantages. Thus, one particular method of introducing genes into a particular eukaryotic cell or plant species may not necessarily be the most effective for another eukaryotic cell or plant species.

Agrobacterium tumefaciens-mediated transfer is a widely applicable system for introducing genes into plant cells because the DNA can be introduced into whole plant tissues, bypassing the need for regeneration of an intact plant from a protoplast. The use of Agrobacterium-mediated expression vectors to introduce DNA into plant cells is well known in the art. See, for example, the methods described by Fraley et al., Biotechnology, 3:629 (1985) and Rogers et al., Methods in Enzymology, 153:253-277 (1987). Further, the integration of the Ti-DNA is a relatively precise process resulting in few rearrangements. The region of DNA to be transferred is defined by the border sequences and intervening DNA is usually inserted into the plant genome as described by Spielmann et al., Mol. Gen. Genet., 205:34 (1986) and Jorgensen et al., Mol. Gen. Genet., 207:471 (1987). Modem Agrobacterium transformation vectors are capable of replication in Escherichia coli as well as Agrobacterium, allowing for convenient manipulations as described by Klee et al., in Plant DNA Infectious Agents, T. Hohn and J. Schell, eds., Springer-Verlag, New York, pp. 179-203 (1985). Further recent technological advances in vectors for Agrobacterium-mediated gene transfer have improved the arrangement of genes and restriction sites in the vectors to facilitate construction of vectors capable of expressing various polypeptide coding genes. The vectors described by Rogers et al., Methods in Enzymology, 153:253 (1987), have convenient multi-linker regions flanked by a promoter and a polyadenylation site for direct expression of inserted polypeptide coding genes and are suitable for present purposes.

In those plant species where Agrobacterium-mediated transformation is efficient, it is the method of choice because of the facile and defined nature of the gene transfer. Agrobacterium-mediated transformation of leaf disks and other tissues appears to be limited to plant species that Agrobacterium tumefaciens naturally infects. Thus, Agrobacterium-mediated transformation is most efficient in dicotyledonous plants.

Few monocots appear to be natural hosts for Agrobacterium, although transgenic plants have been produced in asparagus using Agrobacterium vectors as described by Bytebier et al., Proc. Natl. Acad. Sci. U.S.A., 84:5345 (1987). Therefore, commercially important cereal grains such as rice, corn, and wheat must be transformed using alternative methods. Transformation of plant protoplasts can be achieved using methods based on calcium phosphate precipitation, polyethylene glycol treatment, electroporation, and combinations of these treatments. See, for example, Potrykus et al., Mol. Gen. Genet., 199:183 (1985); Lorz et al., Mol. Gen. Genet., 199:178 (1985); Fromm et al., Nature, 319:791 (1986); Uchimiya et al., Mol. Gen. Genet., 204:204 (1986); Callis et al., Genes and Development, 1:1183 (1987); and Marcotte et al., Nature, 335:454 (1988).

Application of these methods to different plant species depends upon the ability to regenerate that particular plant species from protoplasts. Illustrative methods for the regeneration of cereals from protoplasts are described in Fujimura et al., Plant Tissue Culture Letters, 2:74 (1985); Toriyama et al., Theor Appl. Genet., 73:16 (1986); Yamada et al., Plant Cell Rep., 4:85 (1986); Abdullah et al., Biotechnology, 4:1087 (1986).

To transform plant species that cannot be successfully regenerated from protoplasts, other ways to introduce DNA into intact cells or tissues can be utilized. For example, regeneration of cereals from immature embryos or explants can be effected as described by Vasil, Biotechnology, 6:397 (1988). In addition, “particle gun” or high-velocity microprojectile technology can be utilized. Using such technology, DNA is carried through the cell wall and into the cytoplasm on the surface of small (0.525 μm) metal particles that have been accelerated to speeds of one to several hundred meters per second as described in Klein et al., Nature, 327:70 (1987); Klein et al., Proc. Natl. Acad. Sci. U.S.A., 85:8502 (1988); and McCabe et al., Biotechnology, 6:923 (1988). The metal particles penetrate through several layers of cells and thus allow the transformation of cells within tissue explants. Metal particles have been used to successfully transform corn cells and to produce fertile, stably transformed tobacco and soybean plants. Transformation of tissue explants eliminates the need for passage through a protoplast stage and thus speeds the production of transgenic plants.

DNA can also be introduced into plants by direct DNA transfer into pollen as described by Zhou et al., Methods in Enzymology, 101:433 (1983); D. Hess, Intern Rev. Cytol., 107:367 (1987); Luo et al., Plant Mol. Biol. Reporter, 6:165 (1988). Expression of polypeptide coding genes can be obtained by injection of the DNA into reproductive organs of a plant as described by Pena et al., Nature, 325:274 (1987). DNA can also be injected directly into the cells of immature embryos and the rehydration of desiccated embryos as described by Neuhaus et al., Theor. Appl. Genet., 75:30 (1987); and Benbrook et al., in Proceedings Bio Expo 1986, Butterworth, Stoneham, Mass., pp. 27-54 (1986).

The regeneration of plants from either single plant protoplasts or various explants is well known in the art. See, for example, Methods for Plant Molecular Biology, A. Weissbach and H. Weissbach, eds., Academic Press, Inc., San Diego, Calif. (1988). This regeneration and growth process includes the steps of selection of transformant cells and shoots, rooting the transformant shoots and growth of the plantlets in soil.

The regeneration of plants containing the foreign gene introduced by Agrobacterium tumefaciens from leaf explants can be achieved as described by Horsch et al., Science, 227:1229-1231 (1985). In this procedure, transformants are grown in the presence of a selection agent and in a medium that induces the regeneration of shoots in the plant species being transformed as described by Fraley et al., Proc. Natl. Acad. Sci. U.S.A., 80:4803 (1983). This procedure typically produces shoots within two to four weeks and these transformant shoots are then transferred to an appropriate root-inducing medium containing the selective agent and an antibiotic to prevent bacterial growth. Transformant shoots that rooted in the presence of the selective agent to form plantlets are then transplanted to soil to allow the production of roots. These procedures will vary depending upon the particular plant species employed, such variations being well known in the art.

The immunoadhesins of the present invention may be produced in any plant cell including plant cells derived from plants that are dicotyledonous or monocotyledonous, solanaceous, alfalfa, legumes, or tobacco.

Transgenic plants of the present invention can be produced from any sexually crossable plant species that can be transformed using any method known to those skilled in the art. Useful plant species are dicotyledons including tobacco, tomato, the legumes, alfalfa, oaks, and maples; monocotyledons including grasses, corn, grains, oats, wheat, and barley; and lower plants including gymnosperms, conifers, horsetails, club mosses, liverworts, hornworts, mosses, algaes, gametophytes, sporophytes or pteridophytes.

The present invention also contemplates expressing the immunoadhesins within eukaryotic cells including mammalian cells. One of skill in the art will understand the various systems available for expression of the immunoadhesin in mammalian cells and can readily modify those systems to express the immunoadhesins and chimeric protein receptor molecules, e.g., ICAM-1 molecules, in those cells. In preferred ICAM embodiments, the chimeric ICAM-1, J chain and secretory component molecules of the present invention are placed in a vector pCDM8 which has been previously described by Aruffo, et al., Proc. Natl. Acad. Sci. U.S.A., 84:8573-8577 (1987). The use of the PCDM8 construct is by no means unique and numerous other eukaryotic expression systems systems are available that do not utilize the cog cell expression system and that may be used with the chimeric ICAM-1 and other molecules of the immunoadhesin.

D. Compositions Containing Immunoadhesins

The present invention also contemplates compositions containing an immunoadhesin of the present invention together with plant macromolecules or material. Typically these plant macromolecules or plant materials are derived from any plant useful in the present invention. The plant macromolecules are present together with an immunoadhesin of the present invention for example, in a plant cell, in an extract of a plant cell, or in a plant. Typical plant macromolecules associated with the immunoadhesin of the present invention in a composition are ribulose bisphosphate carboxylase, light harvesting complex pigments (LHCP), secondary metabolites or chlorophyll. The compositions of the present invention have plant material or plant macromolecules in a concentration of between 0.01% and 99% mass excluding water. Other compositions include compositions having the immunoadhesins of the present invention present at a concentration of between 1% and 99% mass excluding water. Other compositions include immunoadhesins at a concentration of 50% to 90% mass excluding water.

The compositions of the present invention may contain plant macromolecules at a concentration of between 0.1% and 5% mass excluding water. Typically the mass present in the composition will consist of plant macromolecules and immunoadhesins of the present invention. When the immunoadhesins of the present invention are present at a higher or lower concentration the concentration of plant macromolecules present in the composition will vary inversely. In other embodiments the composition of plant macromolecules are present in a concentration of between 0.12% and 1% mass excluding water.

The present invention contemplates a composition of matter comprising all or part of the following: a chimeric protein receptor molecule (e.g., an ICAM- 1 molecule), a J chain or a secretory component. These components form a complex and are associated as was previously described. Typically, the composition also contains molecules derived from a plant. This composition may also be obtained after an extraction process yielding functional immunoadhesin and plant-derived molecules.

The extraction method comprises the steps of applying a force to a plant containing the complex whereby the apoplastic compartment of the plant is ruptured releasing said complex. The force involves shearing as the primary method of releasing the apoplastic liquid.

The whole plant or plant extract contains an admixture of immunoadhesin and various other macromolecules of the plant. Among the macromolecules contained in the admixture is ribulose bisphosphate carboxylase (RuBisCo) or fragments of RuBisCo.

Another macromolecule is LHCP. Another molecule is chlorophyll. Other useful methods for preparing compositions containing immunoadhesins include extraction with various solvents and application of vacuum to the plant material. The compositions of the present invention may contain plant macromolecules in a concentration of between about 0.1% and 5% mass excluding water.

The present invention also contemplates therapeutic compositions which may be used in the treatment of a patient or animal. Administration of the therapeutic composition can be before or after extraction from the plant or other transgenic organism. Once extracted the immunoadhesins may also be further purified by conventional techniques such as size exclusion, ion exchange, or affinity chromatography. Plant molecules may be co-administered with the complex.

The present invention also contemplates that the relative proportion of plant-derived molecules and animal-derived molecules can vary. Quantities of specific plant proteins, such as RuBisCo or chlorophyll may be as little as 0.01% of the mass or as much as 99.9% of the mass of the extract, excluding water.

The present invention also contemplates the direct use of the therapeutic plant extract containing immunoadhesins without any further purification of the specific therapeutic component. Administration may be by topical application, oral ingestion, nasal spray or any other method appropriate for delivering the antibody to the mucosal target pathogen.

E. Pharmaceutical Compositions, Formulations, And Routes of Administration

The immunoadhesins described herein can be administered to a patient, preferably in the form of a suitable pharmaceutical composition. Such composition may include components in addition to, or in lieu of, those described above. The composition preferably exhibits either or both of a therapeutic and prophylactic property when administered. The preparation of such compositions can be done according to routine methodologies in the art, and may assume any of a variety of forms, e.g., liquid solutions, suspensions or emulsifications, and solid forms suitable for inclusion in a liquid prior to ingestion. Techniques for the formulation and administration of polypeptides and proteins may be found in Remington's Pharmaceutical Sciences, Mack Publishing Co., Easton, Pa., latest edition. Using these procedures, one of ordinary skill can utilize the immunoadhesins of the invention to achieve success without undue experimentation.

1. Administration Routes

Suitable routes of administration for the invention include, e.g., oral, nasal, inhalation, intraocular, phanyngeal, bronchial, transmucosal, or intestinal administration. Alternatively, one may administer the compound in a local manner, e.g., via injection or other application of the compound to a preferred site of action.

2. Formulations

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes. One or more physiologically acceptable carriers comprising excipients and/or other auxiliaries can be used to facilitate processing of the active compounds into pharmaceutical preparations. Proper formulation is dependent upon the particular route of administration chosen.

For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Suitable carriers include excipients such as, e.g., fillers such as sugars, including lactose, sucrose, mannitol, and/or sorbitol; cellulose preparations such as, e.g., maize starch, wheat starch, rice starch, potato starch, gelatin, gum tragacanth, methyl cellulose, hydroxypropylmethyl- cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

Alternatively, the active ingredient may be in powder form for constitution with a suitable vehicle, e.g., sterile pyrogen-free water, before use.

In addition, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Pharmaceutically compatible salts may be formed with many acids, including but not limited to hydrochloric, sulfuric, acetic, lactic, tartaric, malic, succinic, citric, etc. Salts tend to be more soluble in aqueous or other protonic solvents that are the corresponding free base forms. In solutions, manipulation of pH is also routinely employed for optimizing desired properties.

3. Determining Effective Dosages and Dosage Regimens

Pharmaceutical compositions suitable for use in the present invention include compositions where the active ingredients are contained in an amount effective to achieve an intended purpose, e.g., a therapeutic and/or prophylactic use. A pharmaceutically effective amount means an amount of compound effective to prevent, alleviate or ameliorate symptoms of disease or prolong the survival of the subject being treated. Determination of a pharmaceutically effective amount is well within the capability of those skilled in the art, and will typically assume an amount of between about 0.5 pg/kg/day and about 500 g/kg/day, with individual dosages typically comprising between about 1 nanogram and several grams of immunoadhesin.

For any compound used in the methods of the invention, the therapeutically effective dose can be estimated initially from cell culture assays. For example, varying dosages can be administered to different animals or cell cultures and compared for effect. In this way, one can identify a desired concentration range, and prepare and administer such amount accordingly. Optimization is routine for one of ordinary skill in the art.

The person of skill, in addition to considering pharmaceutical efficacy, also considers toxicity according to standard pharmaceutical procedures in cell cultures or experimental animals, e.g., for determining the LD50 (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and ED₅₀. Compounds which exhibit high therapeutic indices are preferred. The data obtained from these cell culture assays and animal studies can be used in formulating a range of dosage for use in humans. The dosage of such compounds lies preferably within a range of concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. (See e.g., Fingl et al., 1975, in “The Pharmacological Basis of Therapeutics,”.Ch. 1 p.1).

Dosage amount and frequency may be adjusted to provide mucosal levels of immunadhesin sufficient to maintain or provide a pharmaceutical effect, e.g., therapeutic and/or prophylactic. The minimal effective concentration (MEC) will vary for each immunadhesin and immunoadhesin formulation, but can be estimated from in vitro and/or in vivo data. Dosages necessary to achieve MEC will depend on individual characteristics and route of administration. However, assays as described herein can be used to determine mucosal concentrations, which can then be further optimized in amount and precise formulation.

Dosage intervals can also be determined using MEC value. Compounds can be administered using a regimen which maintains mucosal levels above the MEC for 10-90% of the time, 30-90% of the time, or, most preferably, 50-90% of the time.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the immunoadhesin for human or veterinary administration. Such notice, for example, may be the labeling approved by the U.S. Food and Drug Administration for prescription drugs, or the approved product insert. Compositions comprising a compound of the invention formulated in a compatible pharmaceutical carrier may also be prepared, placed in an appropriate container, and labeled for treatment of an indicated condition, e.g. treatment or prophylaxis of a disease mediated by host organism/patient protein receptor molecules.

F. Methods of Treatment and Prevention of ICAM-mediated Afflictions

A patient in need of therapeutic and/or prophylactic immunoadhesin chimeras of the invention, e.g., to counter rhinovirus infection and/or symptoms such as occur with colds, can be administered a pharmaceutically effective amount of desired immunoadhesin, preferably as part of a pharmaceutical composition determined, produced, and administered as described above. These formulations and delivery modalities can vary widely. Described following are preliminary procedures that can be used to deduce effective amounts and toxicity, and which can then be conveniently used to determine treatment and prophylaxis parameters and regimens, both in humans and other animals. These procedures are illustrative only and are not intended to be limiting of the invention. Further, these procedures are routine for one of ordinary skill in the art.

1. Ability of the Immunoadhesin to Reduce Rhinovirus Infectivity in Humans: Dose Escalation Tolerance Study

Immunoadhesins of the invention may be tested, e.g., using randomized controlled trials to determine the effect of administration, e.g., intranasal, of immunoadhesin on infection. Other administration routes can be used. Various assays exist that can be used to monitor effect, e.g., IL-8 response assays assays that evaluate illness symptoms, e.g., cold symptoms caused by rhinovirus infection. These studies can evaluate the extent to which an immunoadhesin taken by a patient subjects can prevent or treat rhinovirus infection. For example, healthy or unhealthy subjects can be administered the immunoadhesin and evalutated over a time course, e.g., in tandem with rhinovirus inoculation and/or illness progression. The clinical protocols used may be based on protocols previously used in evaluation of a recombinant soluble ICAM-1 molecule for efficacy against rhinovirus infection, or modifications thereto (Turner, et. al., JAMA 281:1797-804, 1999).

Male and female subjects of any species, age, health, or disease state can be evaluated The subjects may exhibit a serum neutralizing antibody titer in advance of study, which titer may fluctuate in response to infection and immunoadhesin administration.

The immunoadhesin of the present invention may be formulated as a buffered saline with varying amounts of immunoadhesin within and administered at various intervals to a patient. Single ascending dose and multiple ascending dose studies can be used to evaluate the safety of the immunoadhesin. In each case, one or more subjects may be evaluated at each dosage level, some receiving the immunoadhesin, and one or more optionally receiving placebo. In either study, multiple dosage levels may be evaluated. Dosage levels can vary, but are typically in the nanogram to gram range.

Dosages may be administered over seconds, minutes, hours, weeks, and months, and evaluated for toxicity and/or pharmaceutical effect.

Safety and toxicity may be assessed, e.g., by visual examination of the nasal mucosa for signs of irritation or inflammation. Blood safety evaluations can also be employed according to routine methods and using sensitive assays such as ELISA to determine various blood components, including circulating immunoadhesin and rhinovirus quantities. Naval lavage testing may similarly be done according to routine methodologies.

Routine statistical analyses and calculations may be employed to determine efficacy and toxicity predicted over time courses for single patients and/or for populations of patient-recipients.

Challenge studies as well known in the art can be used to demonstrate that treatment protects against clinical colds and/or reduces cold symptoms after viral challenge, and using commercially available starting materials such virus, cells, and animals. See, e.g., Gwaltney, et. al., Prog. Med. Virol. 39:256-263, 1992.

The following examples illustrate various aspects and embodiments of the disclosed invention. These examples in no way limit the scope of the claimed invention.

### EXAMPLES

1. Construction of ICAM-1 Immunoadhesin Expression Cassettes A cassette encoding ICAM-1 extracellular domains D1 through D5 was prepared by PCR cloning. Specifically, a fragment containing all five extracellular Ig-like domains of ICAM-1 was amplified from plasmid pCDIC1-5D/IgA (Martin, et al. J. Virol. 67:3561-8, 1993) using the following oligonucleotide primers:

| | |
|---|---|
| 5′-TCTGTTCCCAGGAACTAGTTTGGCACAGACATC | (SEQ ID NO: 6) |
| TGTGTCCCCCTCAAAAGTC-3′ | |
| 5′-CATACCGGGGACTAGTCACATTCACGGTCACCT | (SEQ ID NO: 7) |
| CGCGG-3′ | |

These two primers were designed to introduce Spel sites at the 5′ and 3′ ends of the PCR fragment (underlined nucleotides). PCR was performed with Pfu polymerase (Stratagene) to reduce accumulation of errors. The PCR fragment was cloned into the vector PCRScript (Stratagene), and sequenced before fusing to the human IgA2 cassettes (with and without SEKDEL [SEQ ID NO:4] at the carboxy-terminus).

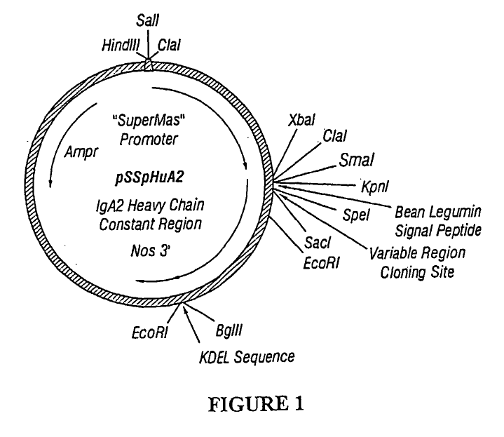

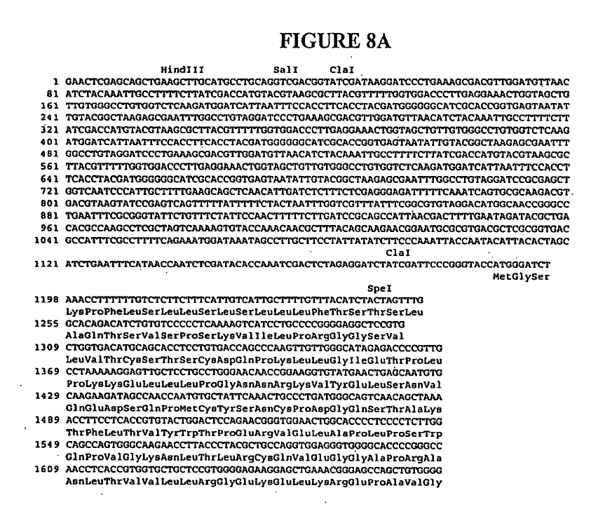

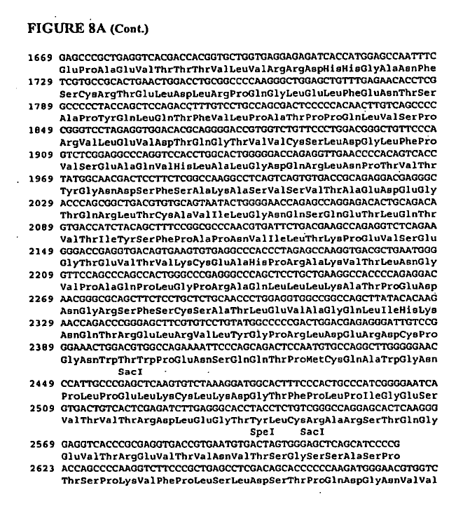

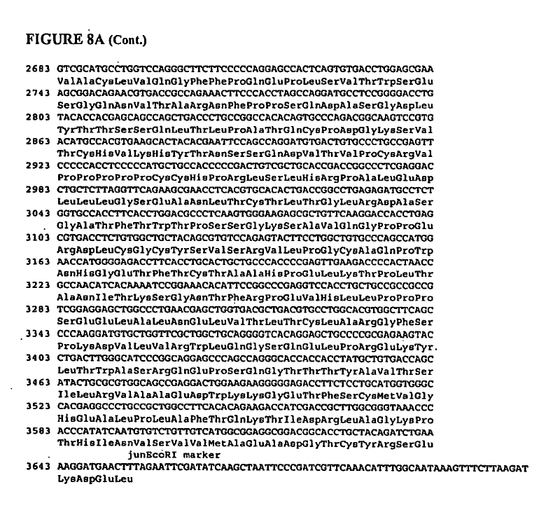

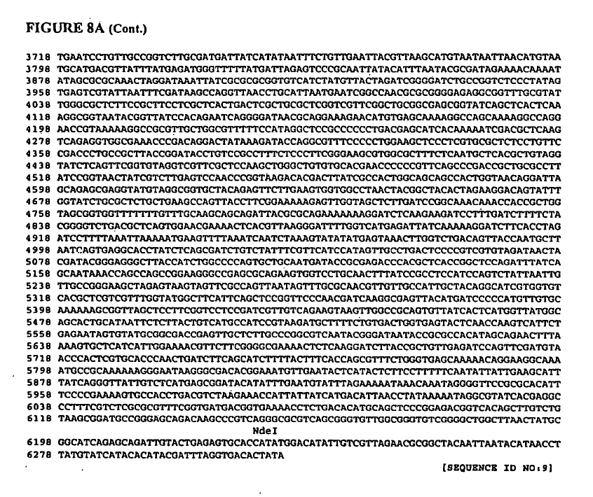

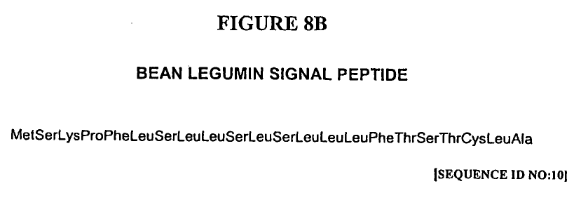

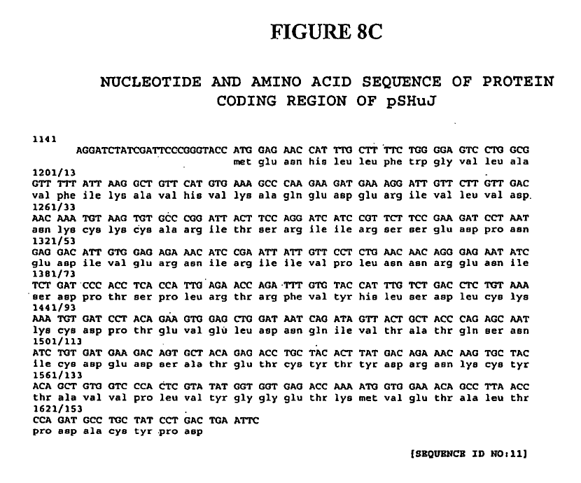

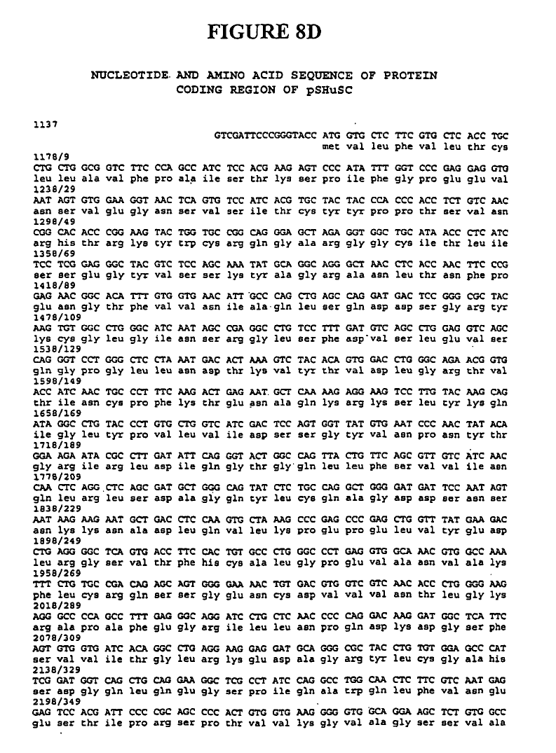

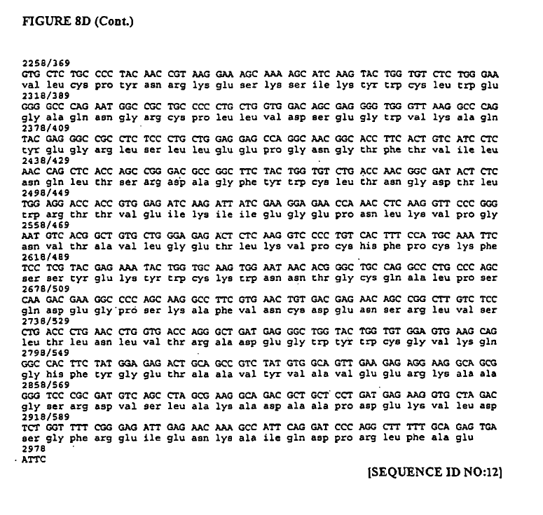

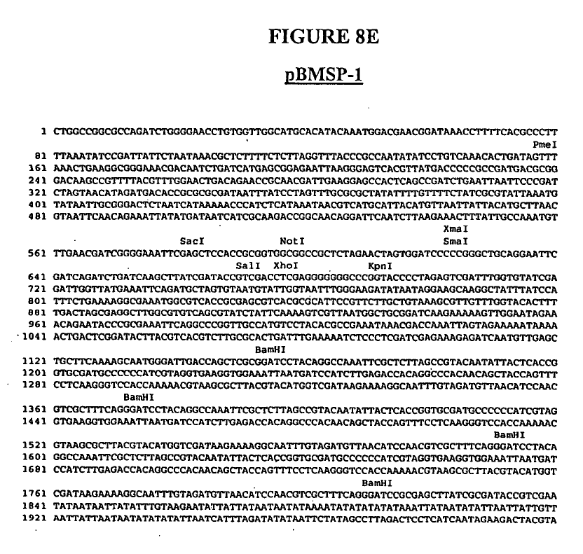

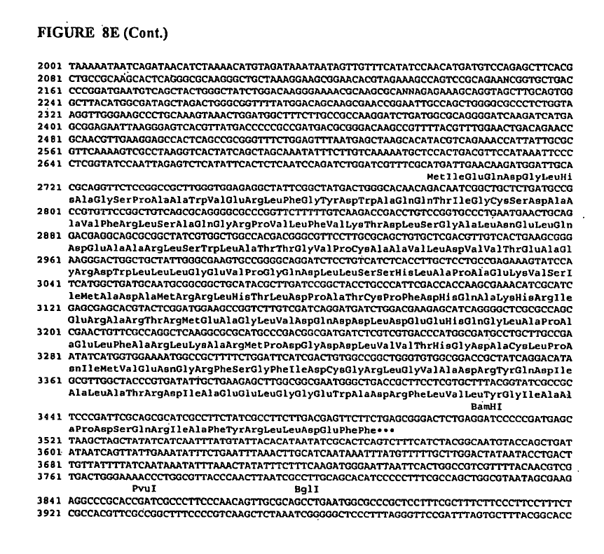

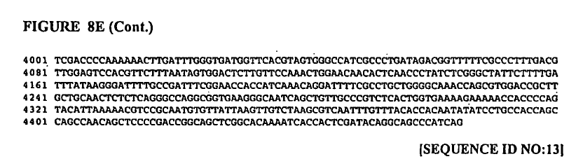

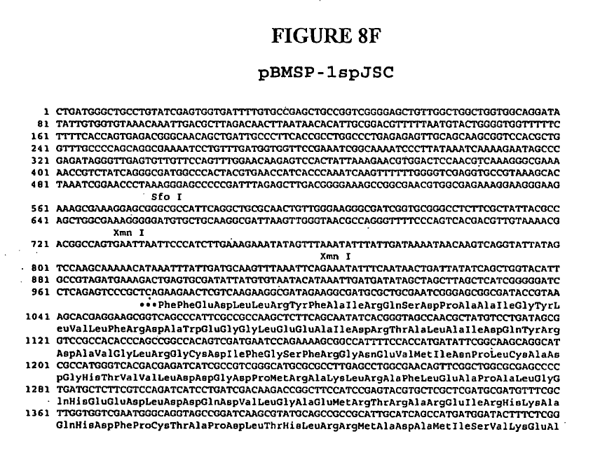

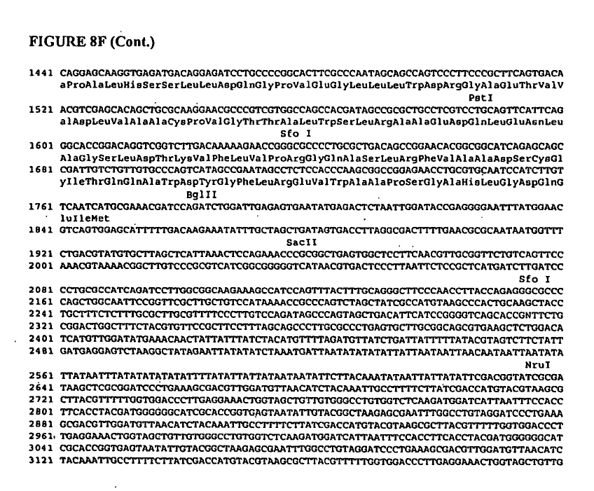

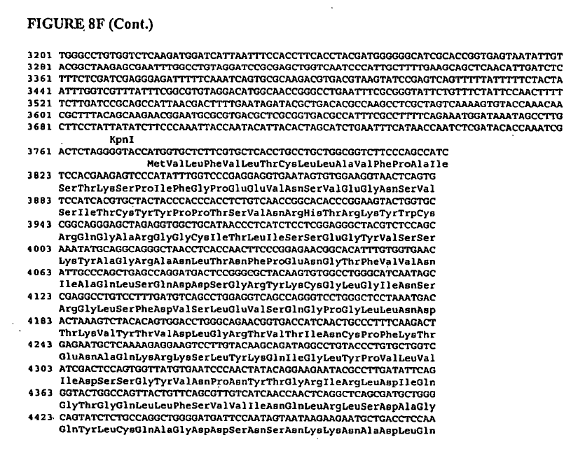

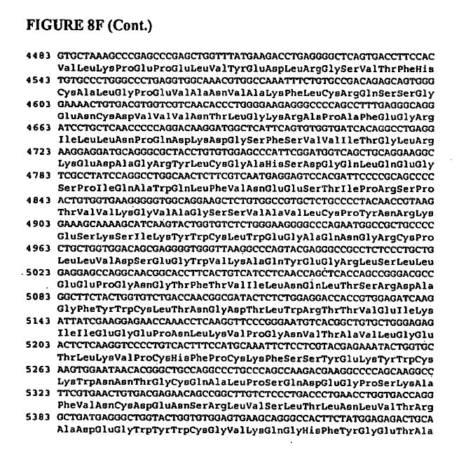

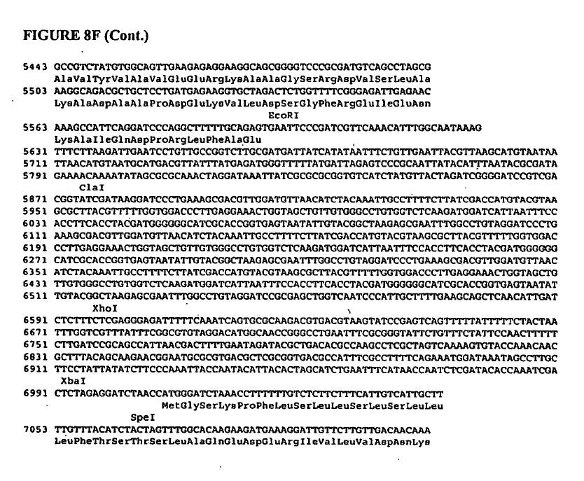

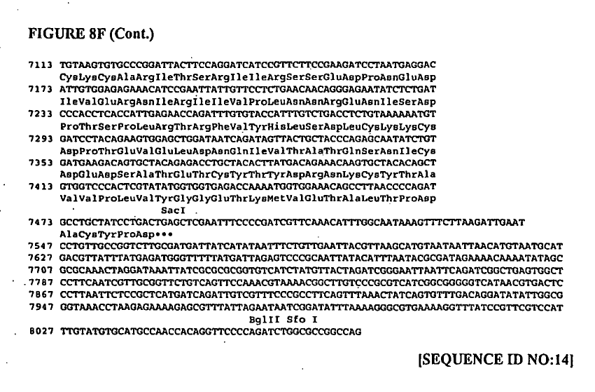

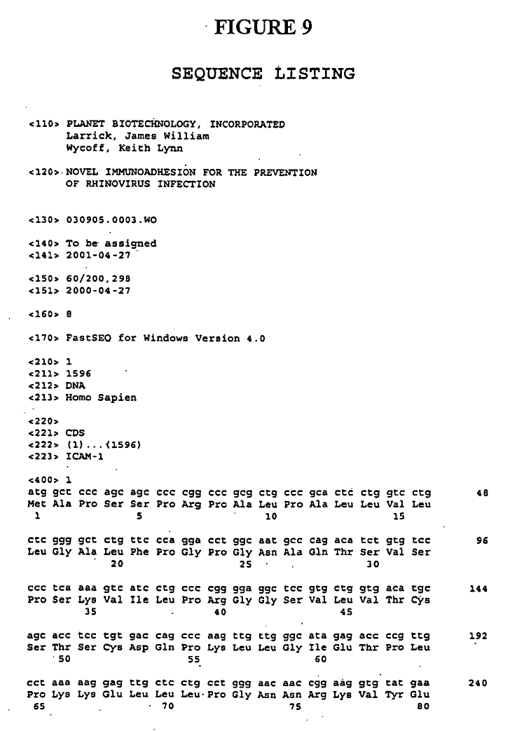

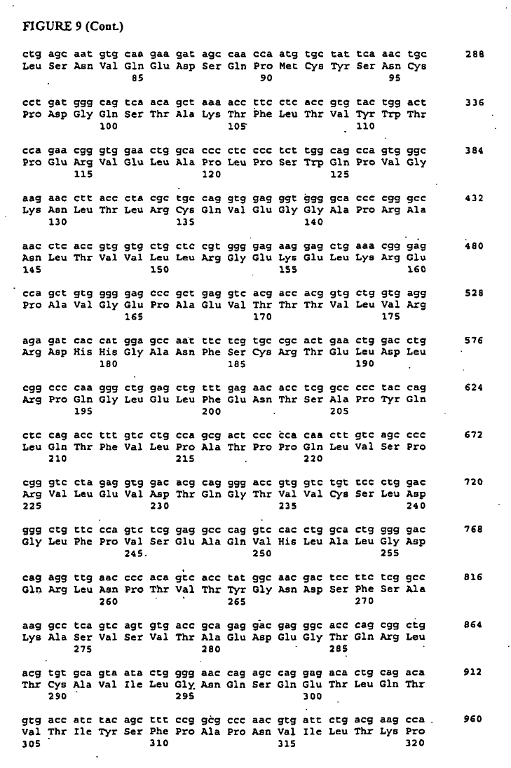

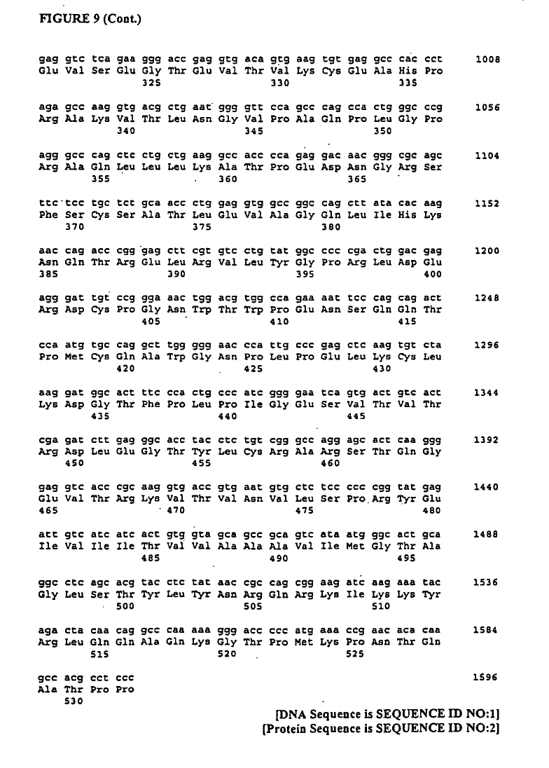

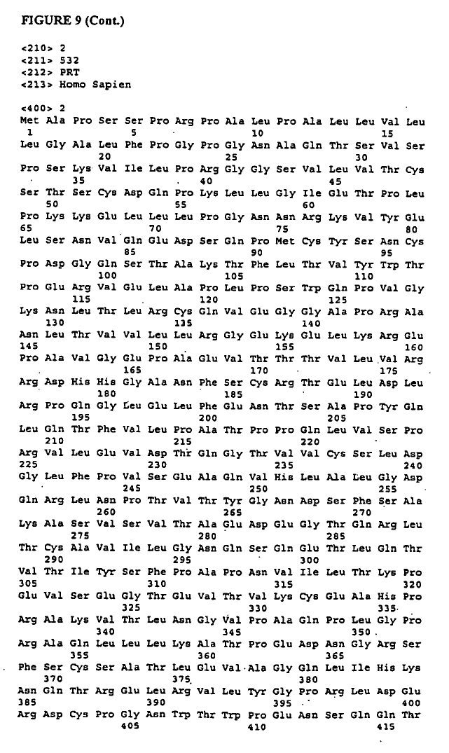

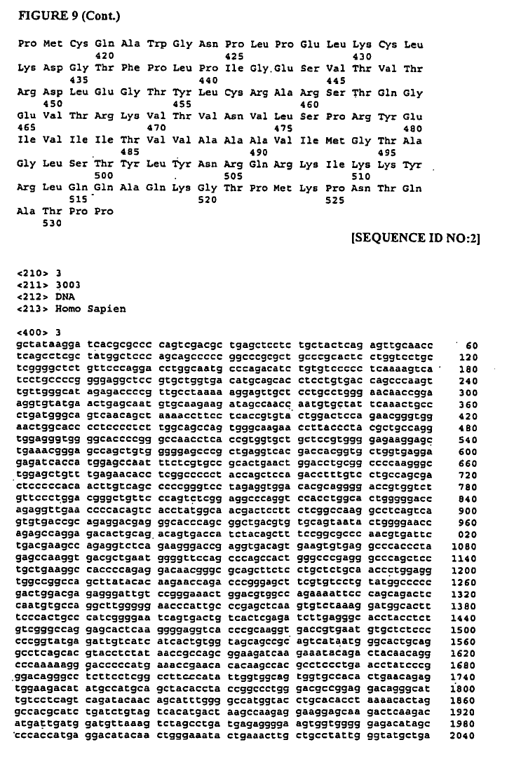

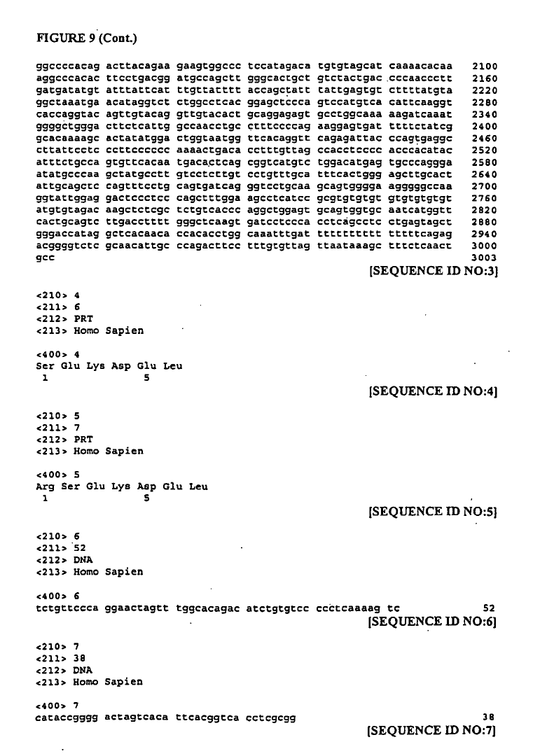

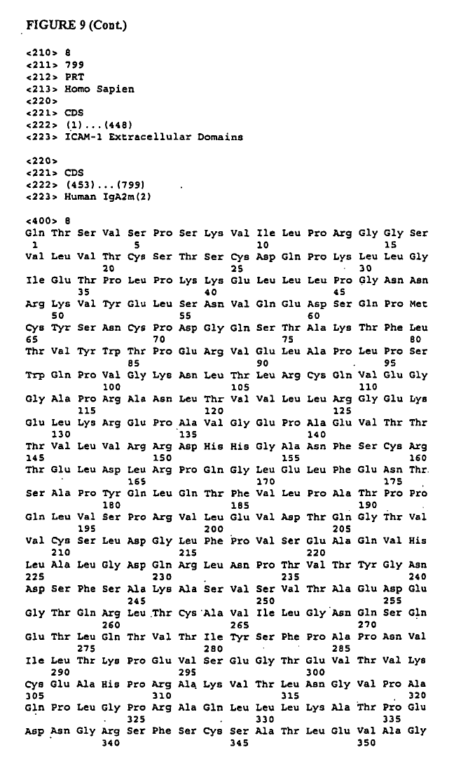

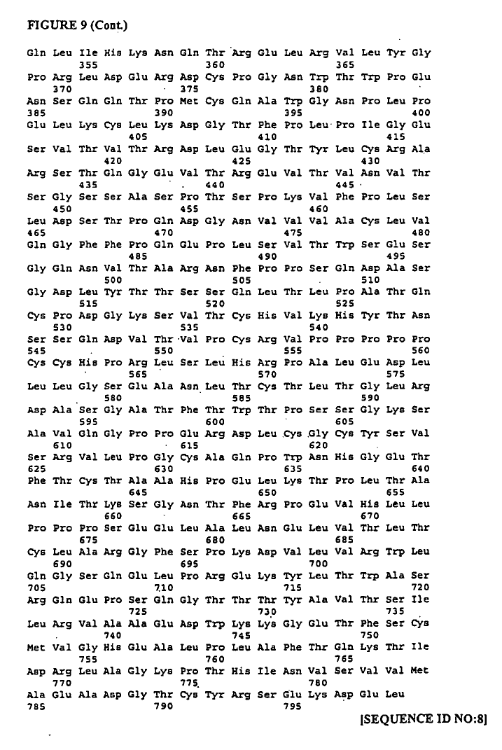

Constructs for the expression in plants of human J chain and secretory component, as well as a human IgA2 heavy chain, were developed. A heavy chain expression cassette vector was made and called pSSpHuA2 (See FIG. 1). It contains sequence encoding a bean legumin signal peptide (Baumlein et al., Nucleic Acids Res. 14 (6), 2707-2720, 1986). The sequence of bean legumin is provided as GenBank Accession No. X03677, and the sequence of the bean legumin signal peptide is SEQ ID NO: 10 (also see FIG. 8) and the IgA2m(2) constant region with SpeI and SacI sites in between, and the SuperMas promoter for driving the expression of a signal peptide and the constant regions of the human IgA2m(2) heavy-chain.

The amplified DNAs encoding the first five domains of human ICAM-1, and the Fc region of human IgA2m(2) were fused in a plant-expression cassette to make a chimeric ICAM-1 molecule expression construct, shown in FIG. 2A. This was done by cloning the fragment encoding the five extracellular domains of ICAM-1 into vector pSSPHuA2 to generate pSSPICAMHuA2. The convenient restriction sites (5′ SpeI and 3′ Spe I) allowed the amplified fragment to be inserted between the signal peptide and the Cα1 domain. In the resulting construct, expression of the chimeric ICAM-1 molecule is under the control of the constitutive promoter “superMAS” (Ni et. al., 1995) and the nos 3′ terminator region.

The resulting chimeric ICAM-1 molecule construct contains no variable region. Upon translation of the mRNA, signal peptide cleavage is predicted to deposit the ICAM-1-heavy chain fusion into the plant cell's endoplasmic reticulum (ER). DNA encoding an ER retention signal (RSEKDEL, SEQ ID NO: 5) was appended to the 3′ end of the heavy-chain in order to boost the expression level of the construct. The amino acid sequence SEKDEL (SEQ ID NO: 4) is the consensus signal sequence for retention of proteins in the endoplasmic reticulum in plant cells. This sequence has been shown to enhance accumulation levels of antibodies in plants (Schouten et al., Plant Molecular Biology 30:781-793,1996). The amino acid sequence of the chimeric ICAM-1 molecule construct is shown in FIG. 2B. The DNA sequence and translational frame of the construct was verified before it was used for particle bombardment.

It has been shown recently that assembly of J chain with IgA takes place in the Golgi apparatus (Yoo et al., J. Biol. Chem. 274:33771-33777, 1999), and so constructions of heavy chain without SEKDEL (SEQ ID NO: 4) have been made as well. The ICAM-1 fragment was cloned into an expression cassette containing the IgA2m(2) constant region without SEKDEL (SEQ ID NO: 4).

2. Expression of Assembled ICAM-1 Immunoadhesin in Plants

A. Immunoadhesin Expression Vectors

The plasmid pSSPICAMHuA2 [SEQ ID NO:9 and FIG. 8A] is 6313 bp in length. Nucleotides 49-1165 represent the Superpromoter (Ni et al., Plant Journal 7:661-676, 1995). Nucleotides 1166-3662 comprise a sequence encoding a human ICAM-1/human IgA2m(2) constant hybrid with linker sequences. A consensus Kozak sequence (Kozak, Cell 44(2):283-92, 1986) is included (nt 1186-1192) to enhance translation initiation, as well as the signal peptide from V. faba legumin (nt 1189-1257; Bäumlein et al., Nucleic Acids Reg. 14(6):2707-2720 (1986). The sequence of the human IgA2m(2) constant region (nt 3663-3633) has been previously published (Chintalacharuvu, et al., J. Imm. 152: 5299-5304, 1994). A sequence encoding the endoplasmic reticulum retention signal SEKDEL [SEQ ID NO:4] is appended to the end of the heavy Chain (nt 3634-3654). Nucleotides 3663-3933 derive from the nopaline synthase 3′ end (transcription termination and polyadenlyation signal; Depicker et al., 1982). The remainder of the plasmid derives from the vector pSP72 (Promega).

The plasmid pSHuJ (FIG. 8C) is 4283 bp in length. Nucleotides 14-1136 represent the Superpromoter (Ni et al., Plant Journal 7:661-676, 1995) and nucleotides 1137-1648 are shown in FIG. 8 [SEQ ID NO:11] and comprise a sequence encoding the human J Chain including the native signal peptide (Max and Korsmeyer, J Imm. 152:5299-5304, 1985) along with linker sequences. A consensus Kozak sequence (Kozak, Cell 44(2):283-92, 1986) is included (nt 1162-1168) to enhance translation initiation. Nucleotides 1649-1902 derive from the nopaline synthase 3′ end (transcription termination and polyadenlyation signal; Depicker et al., J Mol Appl Genet 1(6):561-73, 1982). The remainder of the plasmid derives from the vector pSP72 (Promega).

The plasmid pSHuSC (FIG. 8D) is 5650 bp in length. Nucleotides 13-1136 are derived from the Superpromoter (Ni et al., Plant Journal 7:661-676, 1995), and nucleotides 1137-2981 comprise a sequence encoding the human Secretory Component including the native signal peptide (Krajci, et al., Biochem. and Biophys. Res. Comm 158:783, 1994) along with linker sequences [SEQ ID NO: 12]. A consensus Kozak sequence (Kozak, Cell 44(2):283-92, 1986) is included (nt 1151-1157) to enhance translation initiation. Nucleotides 2982-3236 derive from the nopaline synthase 3′ end, providing a transcription termination and polyadenlyation signal, described in Depicker et al., J Mol Appl Genet 1(6):561-73 (1982). The remainder of the plasmid derives from the vector pSP72 (Promega).

The plasmid pBMSP-1 [SEQ ID NO:13 and FIG. 8E] is derived from pGPTV-KAN. Becker et al., in Plant Molecular Biology 20, 1195-1197, (1992), describe new plant binary vectors with selectable markers located proximal to the left T-DNA border, and the sequences outside of the left and right borders. Nucleotides 18-187 of pBMSP-1 represent the right T-DNA border, and nucleotides 1811-775 represent the superMAS promoter. Nucleotides 2393-2663 represent the NOS promoter (Depicker et al., J Mol Appl Genet 1(6):561-73, 1982), nucleotides 2698-3492 encode the NPTII gene (conferring resistance to kanamycin), and nucleotides 3511-3733 are the polyadenylation signal from A. tumefaciens gene 7 (Gielen et al., Embo J 3:835-46, 1984). Nucleotides 1768-976 encode the NPTII gene, and nucleotides 4317-4464 represent the left T-DNA border.

The plasmid pBMSP-1spJSC [SEQ ID NO: 14 and FIG. 8F] is a derivative of pBMSP, containing both J and SC under control of superpromoter. In this plasmid, nucleotides 1-149 represent the left T-DNA border. Nucleotides 955-733 are the polyadenylation signal from A. tumefaciens gene, nucleotides 1768-976 encode the NPTII gene (conferring resistance to kanamycin), and nucleotides 2073-1803 represent the NOS promoter. Nucleotides 2635-3768 represent the superMAS promoter, nucleotides 3774-5595 encode the Human Secretory component, and nucleotides 5603-5857 represent the NOS polyadenylation signal. Nucleotides 5880-6991 represent the superMAS promoter, nucleotides 7007-7490 encode the Human Joining Chain, and nucleotides 7504-7757 represent the NOS polyadenylation signal. Nucleotides 7886-8057 represent the right T-DNA border.

The plasmid pGPTV-HPT, encoding the enzyme conferring hygromycin resistance, is available commercially from the Max-Planck-Institut für Züchtungsforschung (Germany). It is described by Becker in Plant Molecular Biology 20, 1195-1197 (1992).

B. Plant Transformation and ICAM-1 Immunoadhesin Expression in Plants

The expression cassettes described above were used to produce the assembled immunoadhesin in plants. Plasmids pSSPICAMHuA2, pSHuJ, pSHuSC and pBMSP-1 were co-bombarded into tobacco leaf tissue (N. tabacum cultivar Xanthi) and transformed microcalli were selected on nutrient agar in the presence of kanamycin. Individual microcalli, indicative of independent transformation events, were dissected from the parent tissue and propagated on nutrient agar with kanamycin.

The callus tissues were screened for transgene expression. Callus #7132 was shown to express a chimeric ICAM-1 immunoadhesin and J chain by immunoblotting and PCR (data not shown). This callus did not possess DNA encoding the SC. The callus grew well in culture and, upon accumulation of sufficient mass, #7132 was bombarded again, this time with two of the plasmids described above, PBMSP-1 SpJSC, containing expression cassettes for both the J chain and SC and pGPTV-HPT, containing an expression cassette for the hpt I gene which confers hygromycin resistance. After a period of selection and growth on nutrient agar, several independent transformants were identified, by immunoblotting, that expressed the chimeric ICAM-1 molecule, the J chain and SC in several states of assembly.

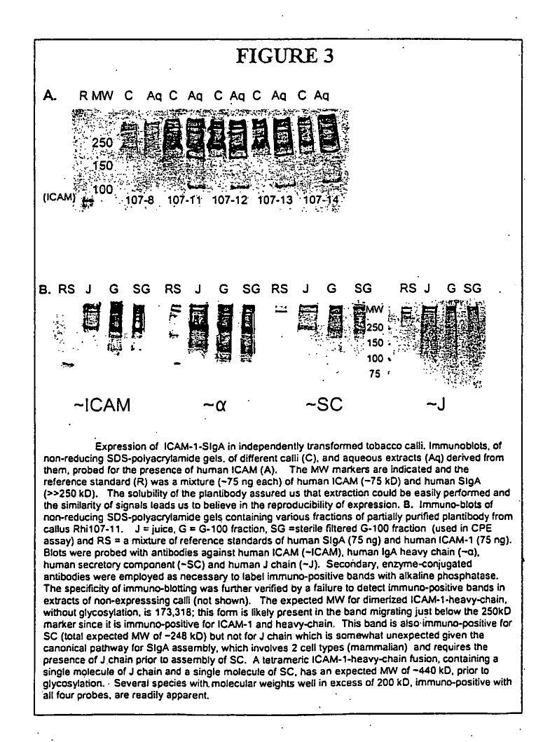

FIG. 3 illustrates the expression of the chimeric ICAM-1 molecule in independently transformed tobacco calli. FIG. 3A shows immunoblots of non-reducing SDS-polyacrylamide gels on which samples containing different transformed tobacco calli (C) and aqueous extracts (Aq) were run and probed for the presence of human ICAM. The solubility of the immunoadhesin assured us that extraction could be easily performed, and the similarity of signals leads us to believe in the reproducibility of expression. FIG. 3B shows immunoblots of nonreducing SDS-polyacrylamide gels containing various fractions of partially purified immunoadhesin from callus Rhi107-11. The blots were probed with antibodies against human ICAM (˜ICAM), human IgA heavy chain (˜α), human secretory component (˜SC) and human J chain (˜J). Secondary, enzyme-conjugated antibodies were employed as necessary to label immuno-positive bands with alkaline phosphatase. The specificity of immuno-blotting was further verified by a failure to detect immuno-positive bands in extracts of non-expressing calli (not shown). The expected MW for a dimerized chimeric ICAM-1 molecule, without glycosylation, is 173,318; this form is likely present in the band migrating just below the 250 kD marker since it is immuno-positive for ICAM-1 and heavy-chain. This band is also immuno-positive for SC (total expected MW of ˜248 kD) but not for J chain which is somewhat unexpected given the canonical pathway for SIgA assembly, which involves 2 cell types (in mammalian) and requires the presence of J chain prior to assembly of SC. A tetrameric immunoadhesin, containing a single molecule of J chain and a single molecule of SC, has an expected MW of ˜440 kD, prior to glycosylation. Several species with molecular weights well in excess of 200 kD, immuno-positive with all four probes, are readily apparent.

Bombardment with DNA-coated microprojectiles is used to produce stable transformants in both plants and animals (reviewed by Sanford et al., Meth. Enz. 217:483-509,1993). Particle-mediated transformation with the vectors encoding the immunoadhesin of the present invention was performed using the PDS-1000/He particle acceleration device, manufactured by Bio-Rad. The PDS-1000/He particle acceleration device system uses Helium pressure to accelerate DNA-coated microparticles toward target cells. The physical nature of the technique makes it extremely versatile and easy to use. We have successfully transformed tobacco with all four components of a secretory IgA simultaneously.

The basic biolistic procedure was performed as follows: A stock suspension of microprojectiles was prepared by mixing 60 mg of particles in 1 ml of absolute ethanol. This suspension was vortexed and 25-50 μl was removed and added to a sterile microcentrifuge tube. After microcentrifuging for 30 seconds the ethanol was removed and the pellet resuspended in 1 ml sterile water and centrifuged for 5 minutes. The water was then removed and the pellet resuspended in 25-50 μl of DNA solution containing a mixture of plasmid DNAs, usually, but not always in equimolar amounts. The amount of plasmid added varied between 0.5 ng and 1 μg per preparation. The following were added sequentially: 220 μl of sterile water, 250 μl of 2.5M CaCl2, and 50 μl of 0.1M spermidine. This mixture was vortexed for at least 10 min and then centrifuged for 5 min. The supernatant was removed and the DNA/microprojectile precipitated in 600 μl of absolute ethanol, mixed and centrifuged 1 min. The ethanol was removed and the pellet resuspended in 36 μl of ethanol. Ten μl of the suspension was applied as evenly as possible onto the center of a macrocarrier sheet made of Kapton (DuPont) and the ethanol was evaporated. The macrocarrier sheet and a rupture disk were placed in the unit. A petri dish containing surface-sterilized tobacco leaves was placed below the stopping screen. The chamber was evacuated to 28-29 mm Hg and the target was bombarded once. The protocol has been optimized for tobacco, but is optimized for other plants as well by varying parameters such as He pressure, quantity of coated particles, distance between the macrocarrier and the stopping screen and flying distance from the stopping screen to the tissue.

Expression cassettes for chimeric ICAM-1 molecules were also assembled in binary vectors for use in Agrobacterium-mediated transformation. An Agrobacterium binary vector designed for expression of both human J chain and human secretory component, as well as kanamycin resistance, was introduced into A. tumefaciens strain LBA4404. The chimeric ICAM/IgA molecule in another binary vector was also used to transform LBA4404. Overnight cultures of both strains were used for simultaneous “co-cultivation” with leaf pieces of tobacco, according to a standard protocol (Horsch et al., Science 227:1229-1231, 1985).

A standard protocol for regeneration of both bombarded and Agrobacterium-transformed tobacco leaf disks was used (Horsch et al., Science 227:1229-1231, 1985). Because transformed plants, regenerated from bombarded tissue, frequently undergo gene-silencing upon maturation, transgenic tobacco plants were prepared via Agrobacterium mediated transformation, which gives a higher yield of expressing, mature plants.

3. Purification of Assembled ICAM-1 Immunoadhesin

The immunoadhesin expressed according to Examples 3 was purified. Calli were grown in large amounts to facilitate the development of extraction procedures. A partial purification schedule provided a stable concentrate, available in a variety of buffer conditions, for investigation of subsequent chromatographic techniques for the further purification of the immunoadhesin (See FIG. 3). Calli were extracted in a juicer, which crushes tissue between two stainless-steel gears, while bathed in a buffer containing sodium citrate (0.6 M, pH 7.4) and urea (final concentration of 2 M). The juice (˜1 ml/g fresh weight) was precipitated, after coarse filtration through cheesecloth, with 0.67 volumes of saturated ammonium sulfate. A green pellet was collected after centrifugation and thoroughly extracted, in a small volume of 50 mM sodium citrate (pH 6.6), with a Dounce homogenizer. After additional centrifugation, a clear brown supernatant was collected and partially purified, during buffer exchange in a de-salting mode, by passage through a Sephadex G-100 column. The desalting/buffer exchange step has allowed preparation of a partially purified concentrate (˜0.2 ml/g fresh weight callus) in a desirable buffer; the G-100 column was eluted with 0.25× phosphate buffered saline. This eluate appeared to be stable for at least 10 days at 2-8° C.

4. The ICAM-1_Immunoadhesin Inhibits Human Rhinovirus Infectivity

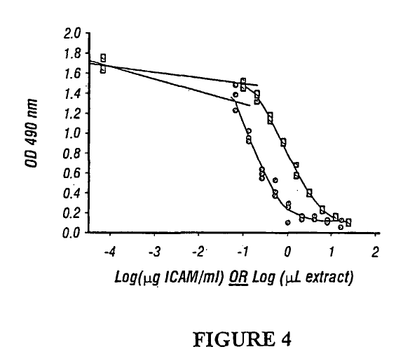

The infectivity of cells by human rhinovirus was demonstrated to be inhibited by the immunoadhesin prepared according to Example 3. Callus extract prepared according to Example 3 successfully competed for binding of an anti-ICAM monoclonal antibody to soluble ICAM-1. FIG. 4 shows the data from an enzyme-linked immunosorbent assay (ELISA). For the assay, 96-well plates were coated with 0.25 μg soluble ICAM-1/ml. The squares represent the increasing concentrations of sICAM and the circles represent the increasing amounts of callus extract (sterile filtered fraction from G-100) used to compete with the adhered ICAM for a constant amount of a mouse (anti-human ICAM) antibody. After washing the wells, adherent mouse antibody was detected with an anti-mouse antibody conjugated to horseradish peroxidase. Adherent enzyme activity was measured at 490 nm, with ortho-phenylene diamine as a substrate. The data (squares, sICAM; circles, Extract) are well described by siginoids of the form OD490=y=y0+a/[1+eˆ−{(x−x0)/b}], where a=y max, y0=y min, b=the slope of the rapidly changing portion of the curve and x0=the value of x at the 50% response level. Relative to soluble ICAM-1, the immunoadhesin extract tested here contains the equivalent of 250 μg ICAM/ml; this is an overestimate due to expected avidity effects of the dimeric and tetrameric assemblies of the ICAM-1-heavy-chain fusions. Thus, this ELISA demonstrated that the immunoadhesin competes with soluble ICAM-1 for binding to an anti-ICAM mAb.

The competitive ELISA allows for quantitative assessment of the recovery of activity by comparing the normalized amounts of various fractions required to give a 50% response. Upon purification, the titer of a immunoadhesin preparation may be expressed as a reciprocal dilution, or the number of milliliters to which a milligram of immunoadhesin must be diluted in order to give a 50% response. This ELISA will facilitate the development of a purification process for the immunoadhesin.

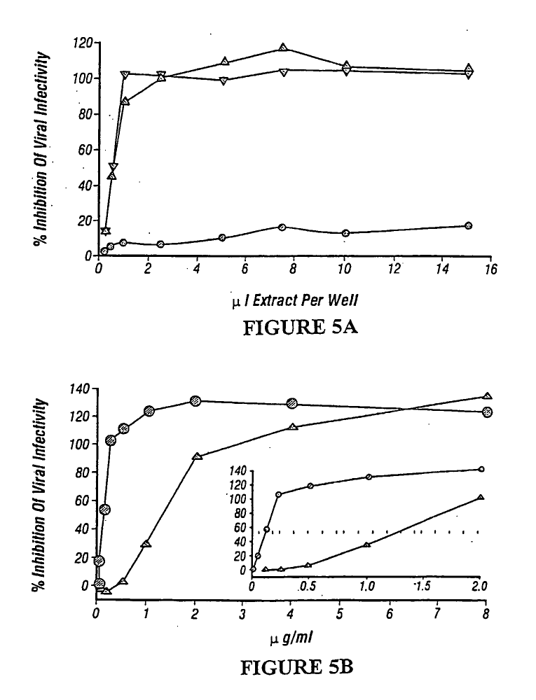

A cytopathic effect assay (CPE) demonstrated the specific ability of the partially purified immunoadhesin to inhibit the infectivity of human cells by human rhinovirus (FIG. 5). Rhinovirus serotype HRV-39 was pre-incubated with human ICAM-1, an ICAM/IgA fusion (gift of Dr. Tim Springer), or with extracts from calli either expressing our ICAM-1/SIgA immunoadhesin or another, different, antibody before plating each of the mixtures with HeLa S3 cells at 33° C. After 3 days, viable cells were fixed and stained with a methanolic solution of Crystal Violet; the optical density at 570 nm provides a proportional measure of cell viability.

Two extracts derived from Rhi107-11, containing the immunoadhesin, clearly inhibited the virus' ability to infect and kill HeLa S3 cells (FIG. 5A, right side-up and upside-down triangles). Because the extracts were only partially purified, we also assayed a similarly prepared extract that contained a human IgA2m(2) directed against Doxorubicin, a chemotherapeutic agent. That extract, containing a similar immunoglobulin with an unrelated binding specificity, was unable to inhibit the infectivity of the rhinovirus and demonstrates that expression of the ICAM-1-heavy-chain fusion confers specificity to the inhibition. The CPE assay demonstrated, as expected, the differential ability of souluble ICAM-1 and an (IC1-5/IgA; Martin, et al., 1993) to inhibit viral infectivity (FIG. 5B). The insert in FIG. 5B is the scale expansion in the range of the IC₅₀ for soluble ICAM-1 (1.35 μg/ml) and for the IC1-5/IgA (0.12 μg/ml; 11.3 fold less).

5. Production and Purification of Immunoadhesins for Clinical and Toxicological Studies

Production of sufficient immunoadhesin for the proposed clinical and toxicological needs is performed by making transgenic tobacco plants. The transgenic plants which express the immunoadhesin (without an ER retention signal) are generated by Agrobacterium-mediated transformation. The absence of an ER retention signal is anticipated to enhance assembly since the nascent SIGA is processed through the entire Golgi apparatus, including, in particular, the trans-Golgi, where SC is covalently linked to dIgA as suggested by pulse-chase experiments (Chintalacharuvu & Morrison, Immunotechnology 4:165-174, 1999). Because Agrobacterium-mediated transformation is much more likely to generate plants with consistent levels of transgene expression, it is likely that progeny of these plants will be used for the production of clinical grade immunoadhesin.

In order to maximize expression levels, and create a true-breeding line, it is desirable to create homozygous plants. The highest producing plants (generation T0) can self-fertilize in the greenhouse before seed is collected. One quarter of the T1 plants are expected to be homozygous. These are grown in the greenhouse and seed samples from several plants are separately germinated on medium containing kanamycin. All the progeny (T2) from homozygous positive plants are expected to be green. Some of the progeny of heterozygous plants are expected to be white or yellowish. Homozygosity is confirmed by back-crossing to wild-type and immunoblotting extracts of the progeny.

Harvesting and processing may be continuously meshed during a production campaign, especially since multiple harvests may be obtained from a single planting, i.e. plants cut to soil level for one harvest are regrown for subsequent harvests. In developing a sense of scale for the production of immunoadhesin it is necessary to decide on the required amount of finished immunoadhesin, account for expression levels (mg immunoadhesin present/kg fresh weight tobacco), know the growth rate of the plants and the expected weight of the average plant, and the overall yield of the purification schedule (set at 20%). Setting the overall need at 3 g of finished immunoadhesin requires preparing for 4 harvests, each with an expected yield of 1 g of finished immunoadhesin.

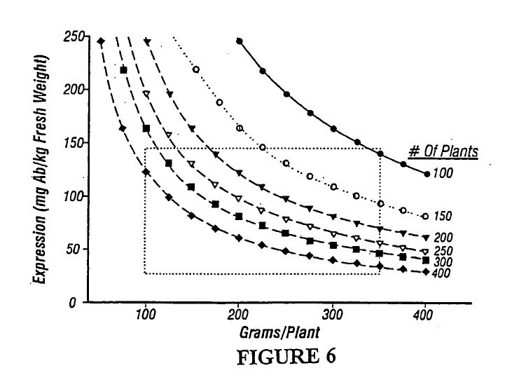

Given these targets and parameters, the necessary number of plants and hence the space requirements for plant growth is determined. FIG. 6 shows an evaluation of the production necessities for making 1 gram of finished Immunoadhesin. In this diagram, the number of plants needed for 1 g of immunoadhesin, at 20% yield, at expected levels of expression and plant weight is illustrated. At different levels of immunoadhesin expression (mg/kg fresh weight) and overall recovery (set at 20%), the weight of each plant, and so the total number of plants, may be determined for a specified production target (1 g/harvest) within a window (dotted square) of reasonable possibilities. The number of required plants decreases, inversely, with the number of specified growth and re-growth periods. The expected biomass production, a function of time and growth conditions, influences the time to harvest and the time between harvests. These growth periods can be adjusted to the realities of the purification schedule by staggering planting and harvesting dates. For example, 1 g of finished immunoadhesin from plants with a reasonable expression level, of 100 mg of immunoadhesin/kg fresh weight, require 250 plants when harvested at a weight of 200 g/plant (˜80 days post germination). At this scale, these plants require about 10 m² of growing space and are harvested twice over 150 days.

Processing 50+ kg of biomass at a time requires several moderately large-scale operations which all have counter-parts in the food-processing industry. These include bulk materials handling, size reduction, juicing and filtration. A Vincent Press and a Durco filtration system are used to efficiently process these quantities. The juicing step employs a proven and simple buffer of sodium citrate and urea. These components buffer the extract, help prevent the oxidation of phenolics and their association with proteins (Gegenheimer, Methods in Enzymology 182:174-193, 1990; Loomis, Methods in Enzymology, 31:528-544, 1974; Van Sumere, et al., The Chemistry and Biochemistry of Plant Proteins, 1975.) and ensure the solubility of the immunoadhesin during a subsequent acid precipitation.

Filtration of acid-insoluble lipid and protein (˜90% of the total) is followed by tangential flow ultrafiltration to concentrate the immunoadhesin and to remove small proteins, especially phenolics. Diafiltration enhances the removal of small molecules and exchanges the buffer in preparation for short-term storage and subsequent chromatography. Either SP-Sepharose (binding at pH 5.0 or below) or Q-Sepharose (binding at pH 5.5 or above) are among the ion-exchanges that can be used for filtering immunoadhesin. They are readily available, scalable, robust and have high capacities. In particular, they are available for expanded-bed formats, which reduce the stringency of prior filtration steps. Cation-exchange chromatography, which can be more selective than anion-exchange chromatography, is used first. The immunoadhesin is purified from the several species of protein potentially present, to the point where at least 95% of the protein is in the form of ICAM-1/IgA, ICAM-1/dIgA or ICAM-1/SIgA, as the presence of di- and tetra-valent ICAM-1 domains are critical for potent anti-viral activity. Purified immunoadhesin is then tested for acceptable levels of endotoxin, alkaloids such as nicotine and for bio-burden. In addition, potency levels (defined by ELISA and CPE assays), protein concentration, pH and appearance are monitored. Subsequently, the stability of the clinical lots of immunoadhesin is determined, to ensure that patients receive fully potent immunoadhesin. Even partially purified extracts have been found to be stable for 10 days when refrigerated. The titer and potency of clinically formulated immunoadhesin (in phosphate-buffered saline), when stored at −20° C., 2-8° C., and at 37° C., over a period of 3 to 6 months, is also tested.

6. The Immunoadhesins Have Plant-Specific Glycosylation

The immunoadhesins produced are analyzed to determine the pattern of glycosylation present. Cabanes-Macheteau et al.,Glycobiology 9(4):365-372 (1999), demonstrated the presence of several glycosyl moieties, typical of plants, on a plant-expressed antibody construct. Their methods are used to demonstrate that the immunoadhesins produced according to Example 1, 2 and 3 have a plant-specific glycosylation pattern. We anticipate that this diversity will also be a source of variability for immunoadhesin. Since crude extracts have been shown to have anti-viral activity in vitro (data not shown), glycosylation, as such, does not appear to affect potency. N-linked glycosylation (FIG. 2 shows that there are fifteen potential sites on the chimeric ICAM-1 molecule alone) probably contributes to the diversity of bands seen in immuno-blots. Immunoadhesin preparations are digested with N-Glycosidase A, before blotting, showing that the difference in banding patterns collapse into fewer, discrete bands. In this way, glycoforms are initially characterized with reducing and non-reducing polyacrylamide gels. In addition, digested and mock-digested fractions are tested in the CPE assay and competition ELISA, demonstrating the effect of N-linked glycosylation on potency and titer in vitro.

7. The ICAM-1 Immunoadhesin Inactivates Human Rhinovirus

The immunoadhesin prepared according to Examples 1, 2 and 3 is assayed for its ability and to inactivate HRV by binding to the virus, blocking virus entry, and inducing the formation of empty virus capsids. To measure binding of the immunoadhesin to HRV, the immunoadhesin is incubated with [³H]leucine-labeled HRV-39 for 30 min and then added to HeLa cells for 1 hr. After washing, cells and bound virus are detached with Triton X-100 and [³H] measured in a scintillation counter.

Inactivation of HRV-39 by incubation with the immunoadhesin is compared with HRV inactivation by sICAM-1. HRV-39 is not directly inactivated to a significant extent (<0.5 log10 reduction in infectivity) by incubation with monomeric sICAM-1, while incubation with IC1-5D/IgA reduced infectivity approximately 1.0 log10 (Arruda, et al., Antimicrob. Agents Chemother. 36:1186-1191, 1992; Crump, et al., Antimicrob. Agents Chemother. 38:1425-7, 1994). In order to test the ability of the immunoadhesin to inactivate HRV-39, 106 50% tissue culture infective doses (TCID₅₀) of HRV-39 are incubated in medium containing a concentration of sICAM-1 or immunoadhesin equal to ten times the IC50 of each molecule for that virus, or in plain medium, for 1 hr at 33° C. on a rocker platform. Each virus-immunoadhesin or virus-medium mixture are then diluted serially in ten-fold dilutions, and the titer determined on HeLa cells in 96-well plates.

The effect of the immunoadhesin on HRV attachment to host cells is tested by inoculating HeLa cells with HRV-39 at a MOI of 0.3 in the presence or absence of the immunoadhesin. Absorbance proceeds for one hour at 4° C., the cells are washed, and media is replaced plus or minus the immunoadhesin. Cells are incubated for ten hours at 33° C. (to allow one round of replication), and virus are harvested by freeze/thawing the cells. The virus is titered on HeLa cells.

ICAM-IGA (IC1-5D/IgA) is more efficient than Sicam-1 at inducing conformational changes in HRV, leading to the formation of empty, non-infectious viral particles (Martin, et al. J. Virol. 67:3561-8, 1993). To examine the ability of the immunoadhesin produced according to Examples 1, 2 and 3 to induce conformational changes in HRV, causing release of viral RNA, purified immunoadhesin is incubated with [³H]leucine-labeled HRV-39 for 30 min and then the virus is overlayed onto a 5 to 30% sucrose gradient. Following centrifugation for 90 min at 40,000 rpm, fractions are collected, [³H] measured, and fractions assessed for infectivity. (Intact HRV sediments at 149S on a sucrose gradient while empty capsids lacking RNA sediments at 75S (Martin, et al. J. Virol. 67:3561-8, 1993)). Due to its increased valence, we expect the ICAM/SIgA immunoadhesin is more efficient at inducing empty non-infectious particles than ICAM-IgA.

The inhibitory effect of purified immunoadhesin on a panel of both major and minor (that do not use ICAM-1 as a receptor) HRV serotypes will be examined using the CPE assay. The ability of ICAM-1 to inhibit HRV infection varies among viral isolates. It has been shown (Crump, et al., Antimicrob. Agents Chemother. 38:1425-7, 1994) that the EC₅₀ for sICAM-1 varies from 0.6 μg/ml to >32 μg/ml when tested on a panel of HRV major receptor serotypes assay using HeLa cells. Our panel includes nine major serotypes (HRV-3, -13, -14, -16, -23, -39, -68, -73, and -80) and the minor receptor serotype HRV-1A.

8. Clinical Studies Demonstrating the Ability of the ICAM-1 Immunoadhesin to Reduce Infectivity in Humans: Dose Escalation Tolerance Study

The immunoadhesin of the present invention is tested in two randomized controlled trials to determine the effect of intranasal administration of the immunoadhesin on infection, IL-8 response, and illness in experimental rhinovirus colds. These two studies evaluate the immunoadhesin taken by subjects before or after rhinovirus inoculation. The clinical protocols used here are based on protocols previously used by in evaluation of a recombinant soluble ICAM-1 molecule for efficacy against rhinovirus infection (Turner, et al., JAMA 281:1797-804, 1999).

A. Subjects

Subjects are recruited from university communities at the University of Virginia, Charlottesville. Subjects are required to be in good health, non-smokers, and between the ages of 18 and 60 years. Subjects are excluded if they have a history of allergic disease or nonallergic rhinitis, abnormal nasal anatomy or mucosa, or a respiratory tract infection in the previous 2 weeks. Pregnant or lactating women or women not taking medically approved birth control are also excluded. In the experimental virus challenge study (Phase I/II, see below), subjects are required to be susceptible to the study virus as evidenced by a serum neutralizing antibody titer of 1:4 or less to the virus, determined within 90 days of the start of the trial.

B. Study Medication

The immunoadhesin of the present invention is formulated as a phosphate-buffered saline (PBS) spray solution containing 2.6 mg/ml. The placebo consists of PBS and is identical in appearance to the active preparation. The solutions are administered using a medication bottle equipped with a metered nasal spray pump. The pump delivers 70 μl of solution containing 183 μg of the immunoadhesin with each spray. The medication is administered as two sprays per nostril, six times daily (at 3-hour intervals) for a total daily dose of 4.4 mg. This is the same dose, in mg protein/day, as was used for soluble ICAM-1 in the tremacamra study infection (Turner, et al., JAMA 281:1797-804, 1999). A mole of the immunoadhesin has about twice the mass as a mole of sICAM-1. However, given the differences in in vitro activity between sICAM-1 and ICAM/IgA fusions, the immunoadhesin is many fold more effective on a molar basis than sICAM-1. Thus, this amount is a conservative calculation of what is necessary. This amount is used, except in the event that the dose escalation study reveals problems at this dose.

C. Study Design

Single ascending dose and multiple ascending dose studies are used to evaluate the safety of the immunoadhesin. In each case, three subjects are evaluated at each dosage level, two receiving the immunoadhesin and one receiving placebo. In the single ascending dose study, four dosage levels are evaluated. The lowest individual dose is half the anticipated dose to be used in the challenge study, and the highest individual dose is twice the anticipated challenge study dose. The dosage levels are as follows: one spray in each nostril (366 μg total), two sprays in each nostril (732 μg total), three sprays in each nostril (1098 μg total), four sprays in each nostril (1464 μg total).

The same dosage levels are used in the multiple ascending dose study. Subjects receive doses every three hours (six times per day) for five days. In both studies subjects are evaluated at each dosage level, staggering the start of each subsequent level until it is clear that there is no acute toxicity at the previous level. All subjects return for a single dose 21 days after the first dose, and then for a follow-up at six weeks (for determination of serum antibody against the immunoadhesin).

A separate group of twelve subjects is given one dose of two sprays in each nostril (732 μg total), and nasal lavage is done at 1, 2, 4, 8 and 16 hours (two subjects at each time point). Washings are assayed at Panorama Research by ELISA for the immunoadhesin in order to calculate its in vivo half-life. The total amount of the immunoadhesin to be used in the dose escalation and half-life determination studies (on a total of 28 subjects) will be approximately 270 mg.

D. Safety Evaluations

In addition to routine adverse event recording, the safety of the immunoadhesin is assessed in three ways. First, prior to the first dose and after the last dose the investigators perform a visual examination of the nasal mucosa, in particular looking for signs of irritation or inflammation. Any visible changes are noted. Second, standard blood safety evaluations are done on samples collected prior to treatment and after the last dose on study days 1, 4, and 8 (and 21 in the multiple ascending dose study). Third, serum samples are saved, frozen, and used to determine if the immunoadhesin is able to pass through the nasal mucosa into the blood. This is accomplished in two ways. First, the presence the immunoadhesin in serum samples is measured by ELISA. In this assay, anti-human IgA antibodies adsorbed to microtiter plates capture any the immunoadhesin in the serum, which are detected by an anti-ICAM antibody. The sensitivity of the assay is determined using normal human serum samples spiked with known concentrations of the immunoadhesin. Alternatively, anti-ICAM antibodies can be adsorbed to plates to capture the immunoadhesin in the serum, that would be detected by anti-IgA. Second, the presence of an immune response to the immunoadhesin is assayed with an ELISA method that uses the immunoadhesin adsorbed to microtiter plates. Any anti-immunoadhesin antibodies in the serum bind, and are detected with anti-human IgG or anti-human IgM. Pre-treatment and post-treatment serum samples are compared, and any change in titer is considered evidence of uptake of the immunoadhesin. If there is any positive evidence of anti-immunoadhesin antibodies, additional assays will be done to distinguish between anti-ICAM-1 and anti-IgA activity.

Patients are screened for the development of an allergic reaction to the immunoadhesin. (In previous studies, there were no episodes of adverse reactions with soluble ICAM applied topically in the nose or plantibodies applied topically in the oral cavity.) Individuals exhibiting symptoms of nasal allergy are tested for anti-immunoadhesin-specific IgE antibodies in nasal lavage fluids using a sensitive two-step ELISA (R & D Systems).

E. Statistical Analysis.

The sample size for these studies is based on previous studies using the rhinovirus challenge model. The sample size planned for the protection studies should be adequate to detect a reduction in the incidence of clinical colds from 75% in the placebo groups to 25% in the active treatment groups at 1-sided levels of α=0.05 and 1-β=0.80. In addition, the sample size should be adequate to detect a change in the total symptom score of 5 units assuming an SD of 5.8 units.

9. Clinical Studies Demonstrating the Ability of the Immunoadhesin to Reduce Infectivity in Humans: Challenge Studies

Challenge studies are used to demonstrate that treatment with the immunoadhesin of the present invention protect against clinical colds or reduce cold symptoms after viral challenge.

A. Challenge Virus.

The challenge virus used for this study is rhinovirus 39 (HRV-39). Rhinovirus type 39 is a major group of rhinovirus that requires ICAM-1 for attachment to cells. The challenge virus pool is safety-tested according to consensus guidelines (Gwaltney, et al., Prog. Med. Virol. 39:256-263, 1992). All subjects are inoculated with approximately 200 median tissue culture infective dose (TCID₅₀). The virus are administered as drops in two inocula of 250 μl per nostril given approximately 15 minutes apart while the subjects are supine.

TABLE 4

| Day | | | | | | | | |
|---|---|---|---|---|---|---|---|---|
| 0 | 1 | 2 | 3 | 4 | 5 | 6 | 7-14 | 21 |

Pre-inoculation study timetable

| | | | | | | | | | |
|---|---|---|---|---|---|---|---|---|---|
| Medications | | 6 doses | 6 doses | 6 doses | 6 doses | 6 doses | | | |
| Inoculation | | hour 4 | | | | | | | |
| Symptom scores | | m/e | m/e | m/e | m/e | m/e | m/e | e | |
| Nasal lavage | | m | m | m | m | m | m | | |
| Serum sample | X | | | | | | | | X |

Post-inoculation study timetable

| | | | | | | | | | |
|---|---|---|---|---|---|---|---|---|---|
| Medications | | 6 doses | 6 doses | 6 doses | 6 doses | 6 doses | | | |
| Inoculation | hour 0 | | | | | | | | |
| Symptom scores | | m/e | m/e | m/e | m/e | m/e | m/e | e | |
| Nasal lavage | | m | m | m | m | m | m | | |
| Serum sample | X | | | | | | | | X |

Note
In both studies on days 1-5, doses are given hours 0, 3, 6, 9, 12, and 15
m = morning
e = evening

B. Study Design

Two randomized rhinovirus challenge studies are performed (see Table 4). The same formulation of the immunoadhesin of the present invention is evaluated in pre-inoculation and post-inoculation studies. In both studies, medication is administered as six doses each day for five days. Subjects are randomly assigned to receive either the immunoadhesin or matching placebo at the time of enrollment into each study. The study is blinded and all clinical trial personnel, subjects, and employees of Panorama Research remain blinded until all data are collected.

In the pre-inoculation study, medications are started four hours (two doses) prior to viral challenge. The virus challenge is administered one hour after the second dose of the immunoadhesin (or placebo) and the four remaining doses of study medication for the first day are given as scheduled. In this study eighteen subjects receive the active treatment and eighteen subjects receive placebo.

In the post-inoculation study, medications begin 24 hours after virus challenge. This timepoint was chosen because it has been used in other studies of protection from virus challenge, and because cold symptoms are clearly present (Harris & Gwaltney, Clin. Infect. Dis. 23:1287-90, 1996). Virus challenge in this study is administered in the morning of study day 0 approximately 24 hours prior to the first dose of study medication on the morning of study day 1. In this study, 36 subjects receive the active treatment and 18 subjects receive placebo.

Subjects are isolated in individual hotel rooms from study day 0 (the day of virus challenge) to study day 6. On each of these days a symptom score and a nasal lavage for virus isolation are done in the morning prior to the first dose of medication and a second symptom score is done each evening. On study day 6, subjects are released from isolation but continue to record symptom scores each evening through day 14. The subjects return to the study site on study day 21, when a final serum sample for detection of anti-immunoadhesin antibodies will be collected. The total amount of immunoadhesin to be used in the two virus challenge studies (on a total of 54 subjects) is approximately 1200 mg.

C. Viral Isolation

Virus shedding is detected by virus isolation in cell culture. Nasal wash specimens are collected by instillation of 5 ml of 0.9% saline into each nostril. This wash is then expelled into a plastic cup and kept chilled for one to two hours until it is processed for viral cultures. Immunoadhesin is removed from the specimens by treatment with anti-ICAM-1 antibody adsorbed to an agarose support (Affi-Gel 10, Bio-Rad Laboratories, Hercules, Calif.). A portion of each processed specimen is stored at −80° C., and another portion is inoculated into two tubes of HeLa-1 cells, a HeLa cell line enriched for the production of ICAM-1 Arruda, et al., J. Clin. Microb. 34:1277-1279, 1996). Rhinovirus are identified by the development of typical cytopathic effect. Subjects with a positive viral culture on any of the postchallenge study days are considered infected. Viral titers in the specimens stored at −80° C. are determined by culturing serial ten-fold dilutions in microtiter plates of HeLa-1 cells.

Antibody to the challenge virus are detected by serum neutralizing titers done using standard methods Gwaltney, et al, Diagnostic Procedures for Viral Rickettsial and Chlamydial Infections, p. 579-614, American Public Health Association). Serum specimens for antibody testing are collected during screening, immediately prior to virus challenge (acute), and again 21 days later (convalescent). Subjects with at least a four-fold rise in antibody titer to the challenge virus when the convalescent serum sample is compared with the acute serum sample are considered infected.

D. Evaluation of Illness Severity

Illness severity is assessed as previously described (Turner, et al., JAMA 281:1797-804, 1999). Symptom scores are recorded prior to virus challenge (baseline) and twice each day at approximately twelve-hour intervals for the next 6 days. On study days 7 through 14 each subject records his/her symptom score once per day in the evening. At each evaluation, subjects are asked to judge the maximum severity of the following eight symptoms in the interval since the last symptom evaluation: sneezing, rhinorrhea, nasal obstruction, sore throat, cough, headache, malaise, and chilliness. Each symptom is assigned a severity score of 0 to 3 corresponding to a report of symptom severity of absent, mild, moderate, or severe. If symptoms are present at baseline, the baseline symptom score will be subtracted from the reported symptom score. The higher of the two daily evaluations are taken as the daily symptom score for each symptom. The daily symptom scores for the eight individual symptoms are summed to yield the total daily symptom score. The total daily symptom scores for the first 5 days after virus challenge (study days 1-5) are summed and on the evening of study day 5, all subjects are asked, “Do you feel you have had a cold?” Subjects who had a total symptom score of at least 6 and either at least three days of rhinorrhea or the subjective impression that they had a cold are defined as having a clinical cold.

The weight of expelled nasal secretions is determined on days 1-7 by providing all subjects with packets of preweighed nasal tissues. After the tissues are used they are stored in an airtight plastic bag. Each morning the used tissues, together with any unused tissues from the original packet, are collected and weighed.

E. IL-8 Assay.

Recent studies have suggested that the host inflammatory response, particularly interleukin 8 (IL-8), may play a role in the pathogenesis of common cold symptoms due to rhinovirus infection. Concentrations of IL-8 in nasal lavage are determined with a commercially available ELISA (R&D Systems, Minneapolis, Minn.) as previously described (Turner, et al., JAMA 281:1797-804, 1999).

F. Safety Evaluations

The same evaluations are, done in the challenge study as in the dose escalation study described in Example 8.

G. Statistical Analysis

Statistical analysis is performed similarly as to that described for the dose escalation study described in Example 8.

The foregoing examples and discussion, while predominantly addressed to ICAM-1 immunoadhesins, can be readily adapted by one of skill to achieve and implement the use of other types of immunoadhesins active against other types or subtypes of virus and bacterial pathogens. The following examples illustrate anti-bacterial immunoadhesin embodiments making use of the anthrax toxin receptor (ATR) as receptor protein.

10. Construction of ATR Immunoadhesin Expression Cassettes

A cassette encoding a portion of the extracellular domains of human anthrax toxin receptor (ATR) is prepared by PCR cloning. Specifically, a fragment of 523 bp, encoding amino acids 44-216 (the so-called von Willebrand factor type A domain) is amplified from plasmid ATR (Bradley et al., 2001), or from plasmid TEM8 (St Croix et al., 2000) using the following oligonucleotide primers:

| | |
|---|---|
| 5′- [symbol] AAATCA | (SEQ ID NO: 91) |
| GG-3′ | |
| 5′-GAGCTCAAAATTGAGTGGATGATGCCT | (SEQ ID NO: 92) |
| TGCAGAG -3′ | |

The second primer (SEQ ID NO: 92) is designed to introduce a Sac I site at the 3′ end of the coding region of the ATR extracellular domain (solid underline). PCR is performed with Pfu polymerase (Stratagene) to reduce accumulation of errors. A second fragment of 124 bp, which includes a 5′ untranslated region and a plant signal peptide, is amplified from plasmid 8ATG-TOPO#4 (which is a PCR clone of a plant-optimized 5′ untranslated region and signal peptide in the Invitrogen cloning vector pCR4-TOPO), using the following oligonucleotide primers:

| | |
|---|---|
| 5′-GGTACCACTTCTCTCAATCCAACTTTC-3′ | (SEQ ID NO: 93) |
| 5′- [symbol] AGCCAA | (SEQ ID NO: 94) |
| ACTAGTAGAGGTGAACAAAAGC-3′ | |

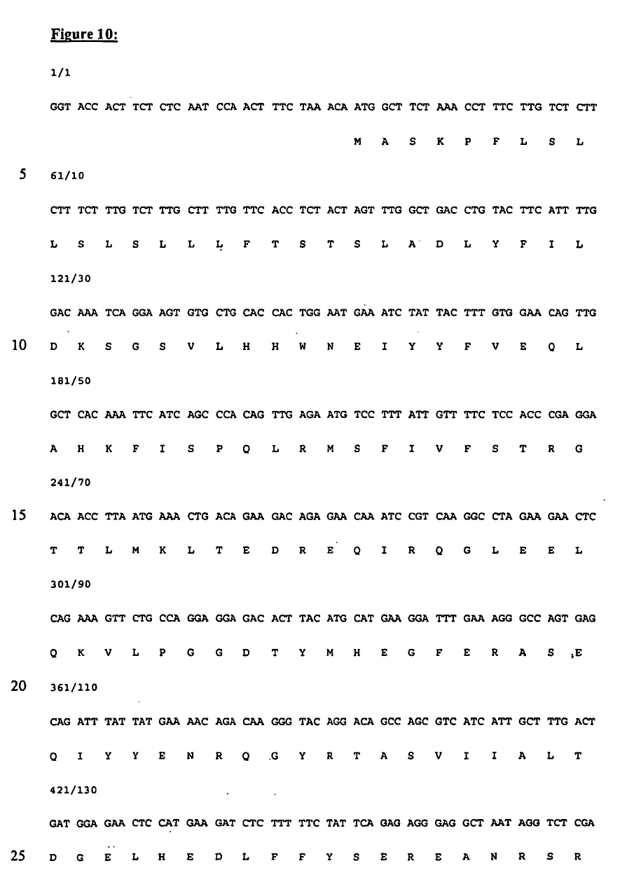

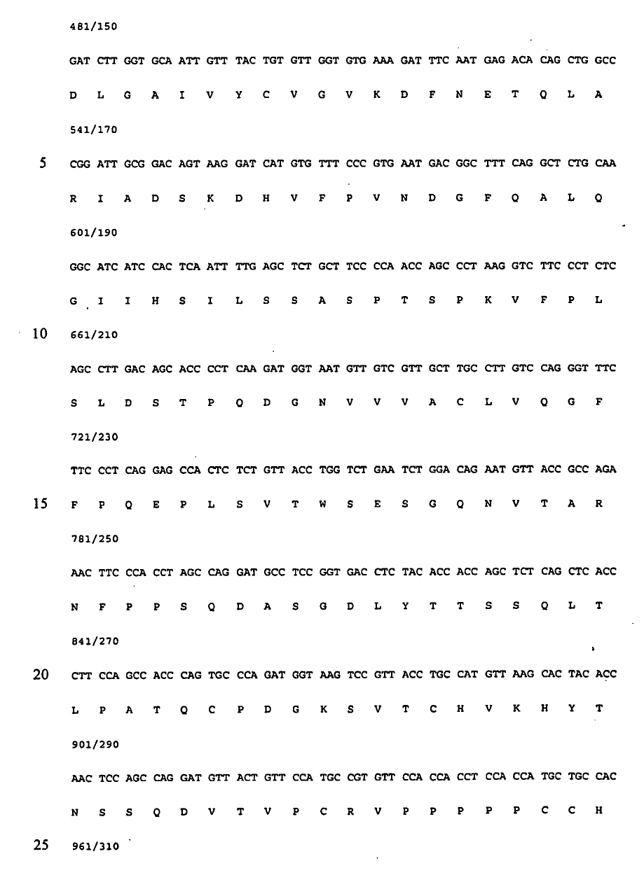

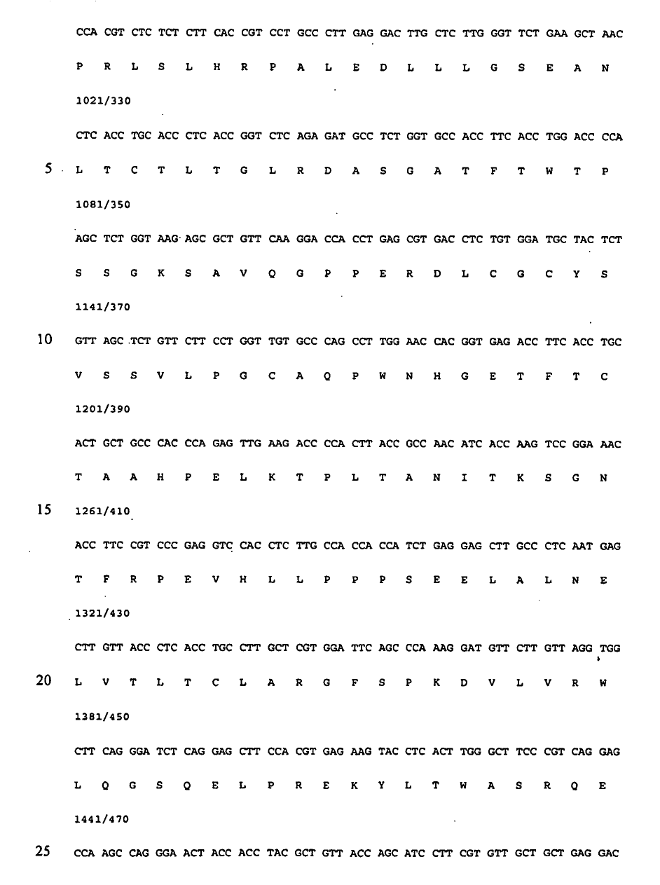

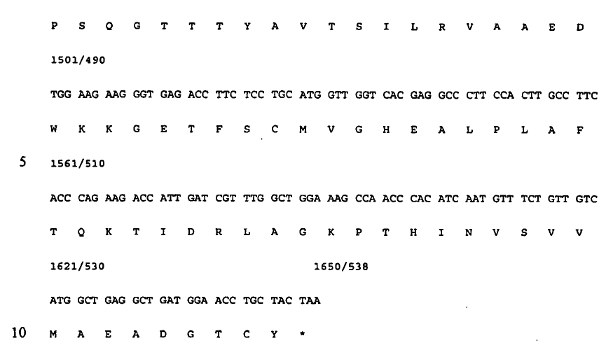

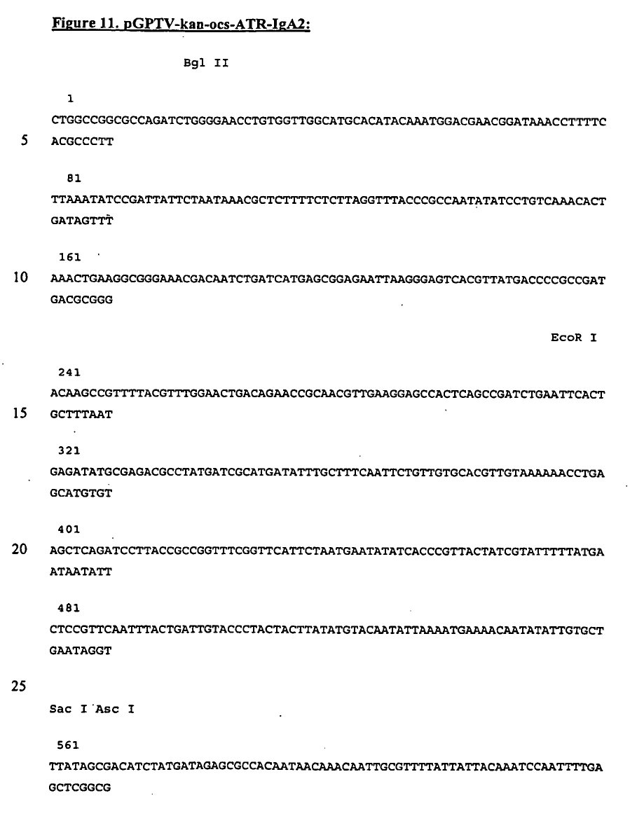

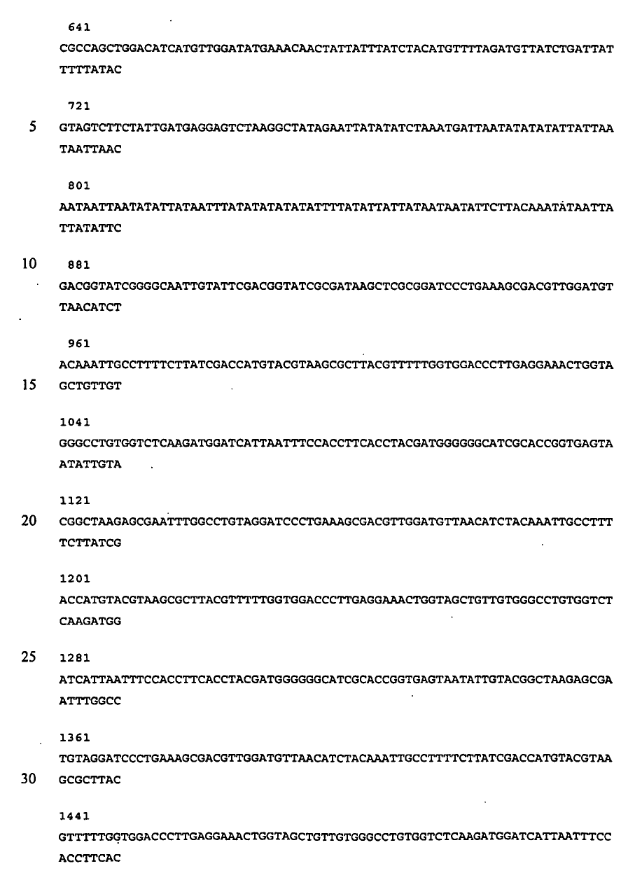

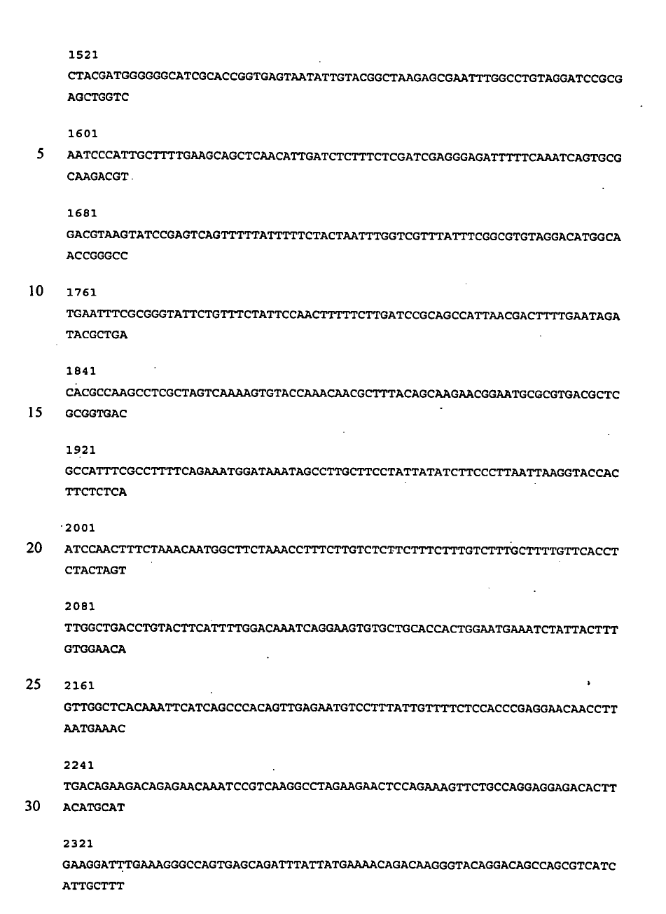

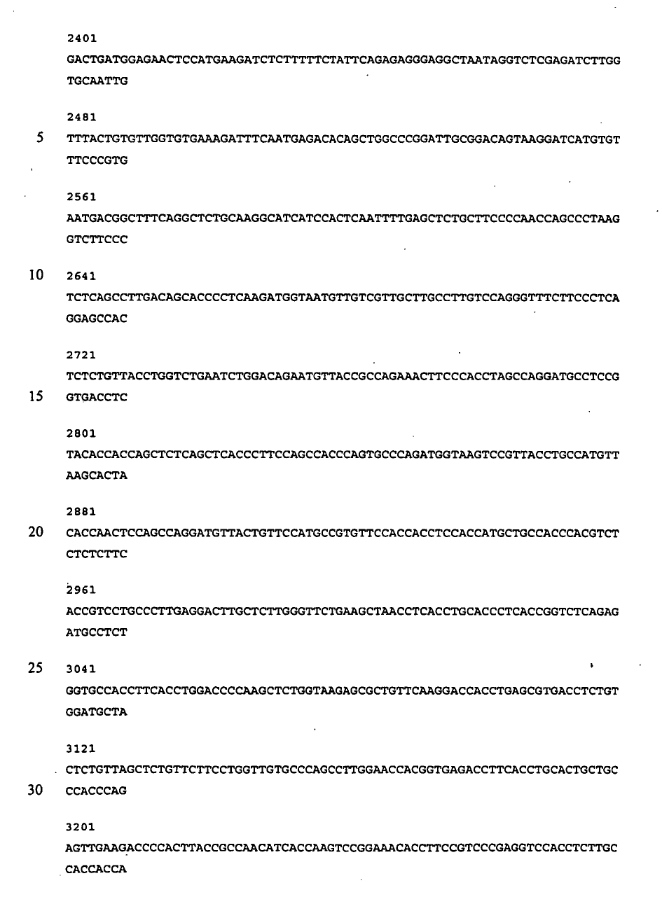

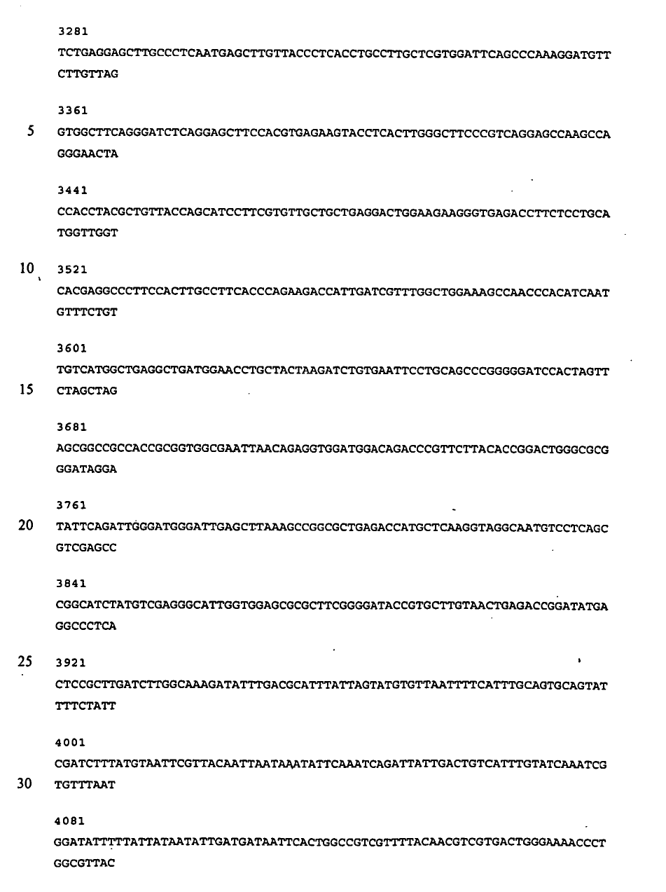

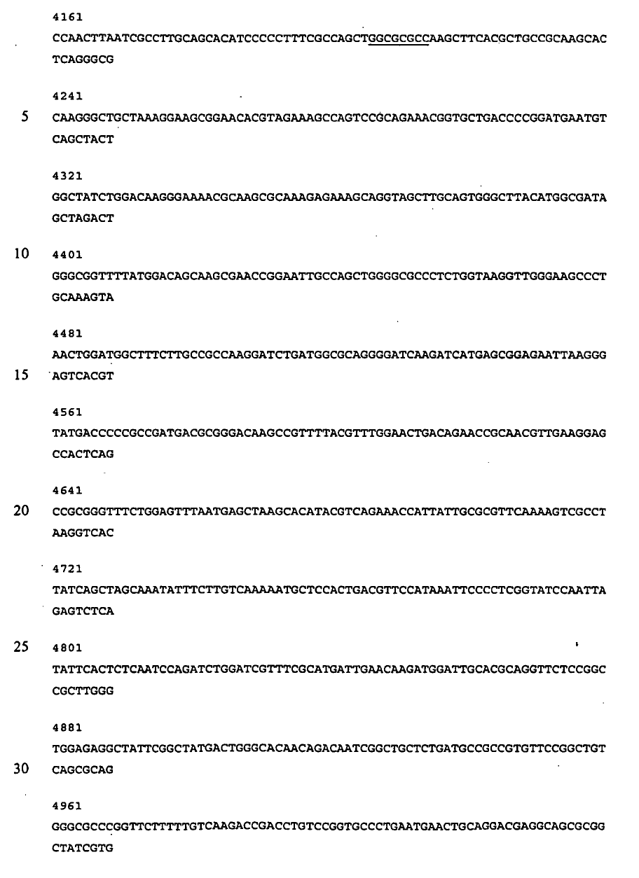

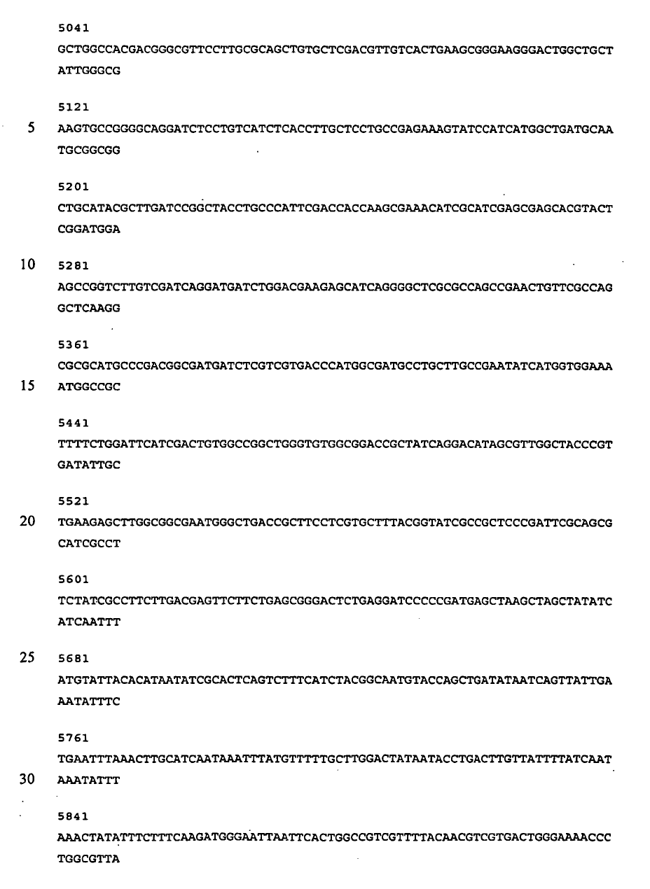

The first primer (SEQ ID NO: 93) is designed to introduce a Kpn I site at the 5′ end of the PCR fragment (solid underline). The two PCR fragments have 20 nt of complementary sequence (dotted underlines). The two PCR fragments are mixed together, and a fragment of 626 bp is amplified using SEQ ID NO: 93 and SEQ ID NO: 92. The resulting PCR fragment is cloned into the vector PCRScript (Stratagene), and sequenced before cloning between Kpn I and Sac I sites in the vector pMSP-coICAM, resulting in plasmid pMSP-ATR-IgA2. This results in a genetic fusion of the extracellular domain of ATR and the constant region of human IgA2. This human IgA2 constant region has been synthesized to use codons optimal for expression in tobacco cells. The full nucleotide and amino acid sequence of the ATR-IgA2 fusion (the immunoadhesin) is shown in FIG. 10. In the resulting construct, expression of the chimeric ATR-IgA2 molecule is under the control of the constitutive promoter “superMAS” (Ni et al., 1995) and the ags 3′ terminator region.

The entire expression cassette (promoter+ATR-IgA2+terminator) is removed from pMSP-ATR-IgA2 with the restriction enzyme Asc I, and cloned into the binary Agrobacterium Ti plasmid vector pGPTV-kan-ocs, resulting in plasmid pGPTV-kan-ocs-ATR-IgA2. The vector pGPTV-kan-ocs is derived from pGPTV-kan (Becker et al., 1992), which was modified in the following manner. The sequence between the Eco RI and Hind III sites of pGPTV-kan, including the entire uid A gene, was removed and replaced with the ocs 3′ terminator region (MacDonald et al., 1991) oriented toward the npt II gene, plus the restriction sites for Asc I and Sac I. The purpose of this terminator adjacent to the right border of the T-DNA is to eliminate transcriptional interference with the transgene due to transcription originating in the plant DNA outside of the right border (Ingelbrecht et al., 1991).

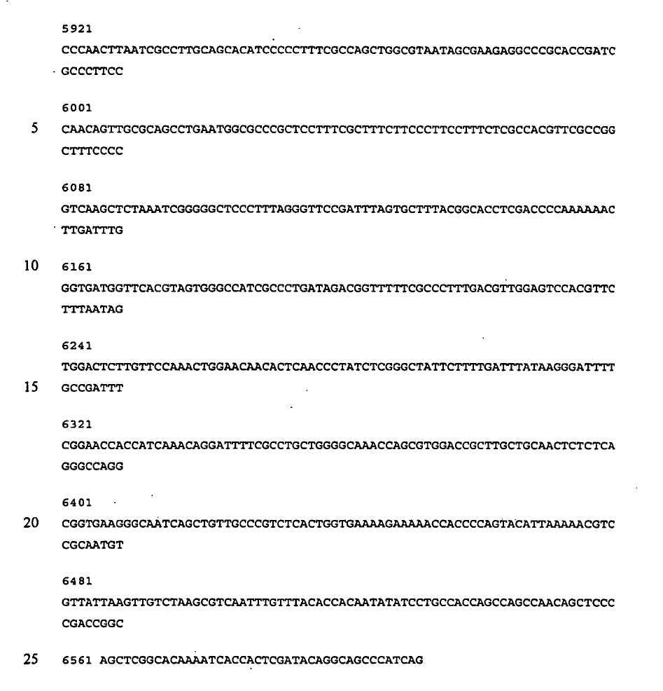

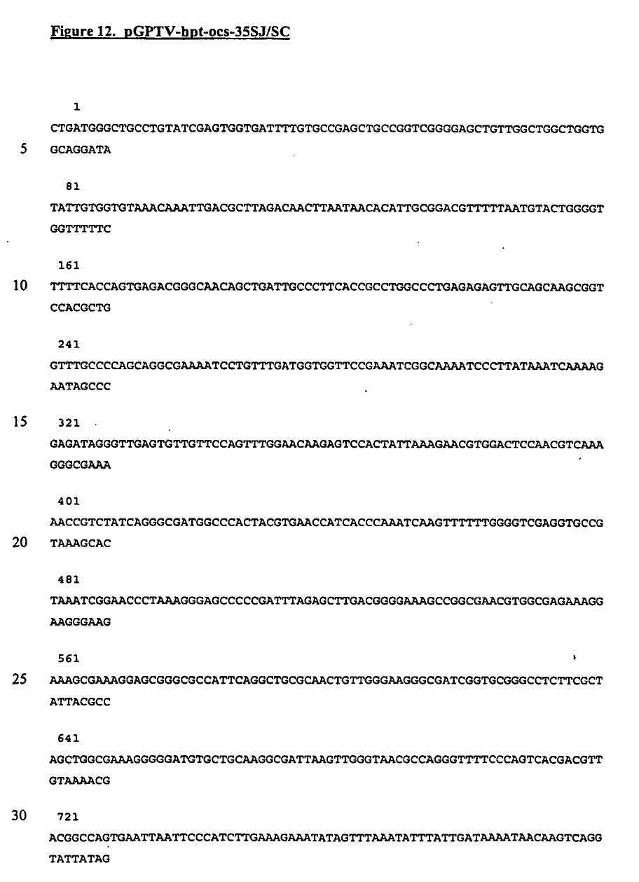

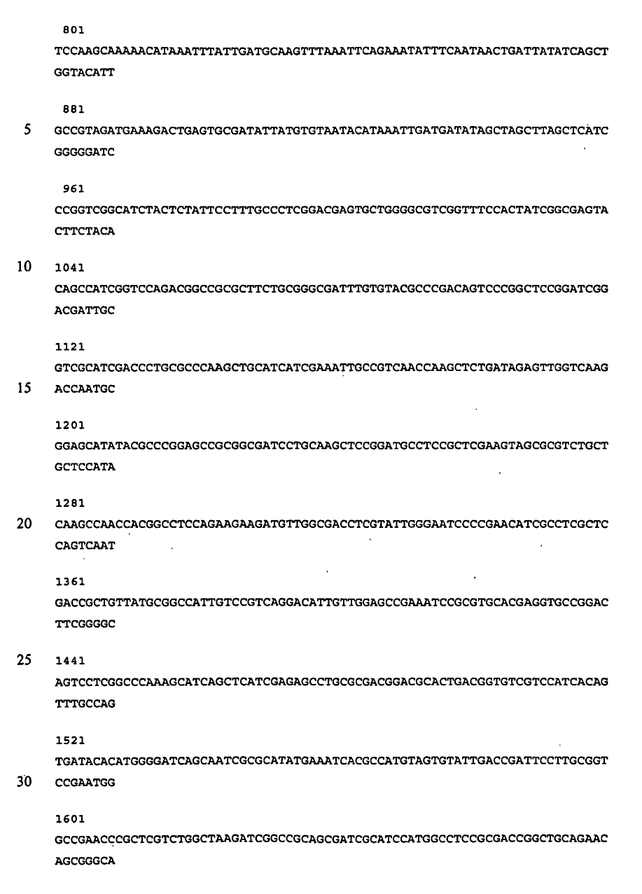

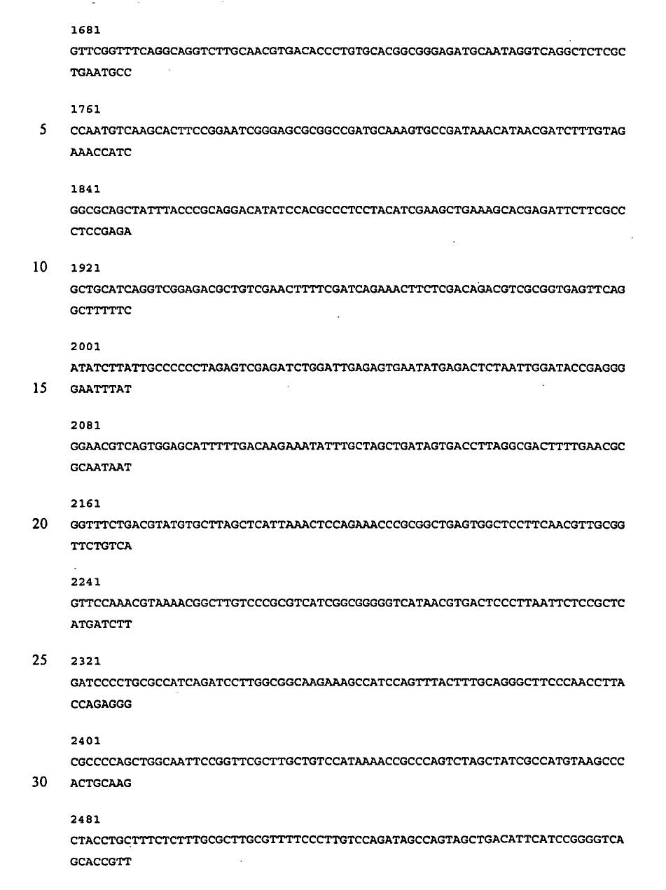

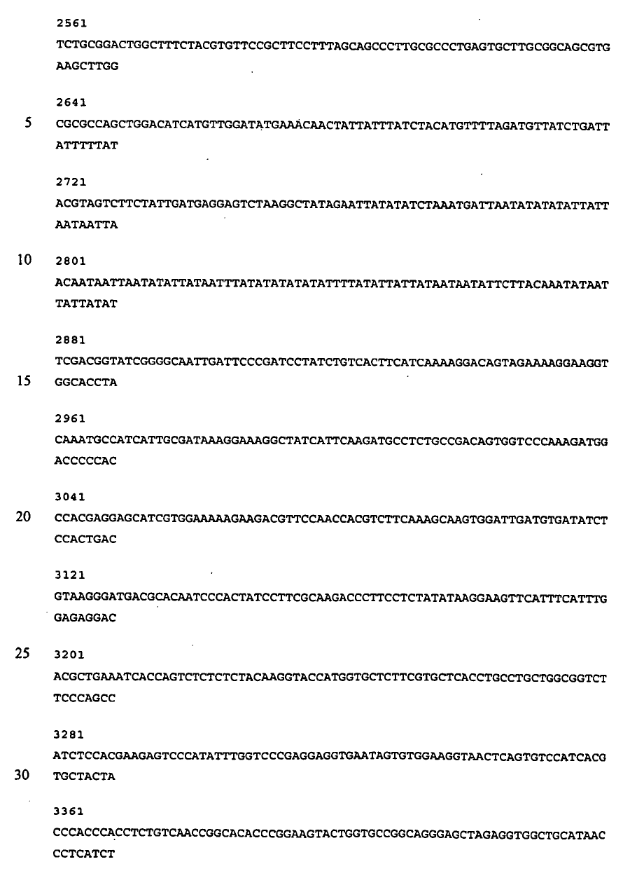

Sequence between the T-DNA borders of the plasmid pGPTV-kan-ocs-ATR-IgA2 is shown in FIG. 11. Sequence outside the left and right borders are as described (Becker et al., 1992). Nucleotides 18-187 represent the right T-DNA border. Nucleotides 311-630 represent the ocs 3′ terminator region. Nucleotides 927-1976 represent the superMAS promoter. Nucleotides 1990-2017 represent a 5′ untranslated region from the Nicotiana sylvestris psaDb gene (Yamamoto et al., 1995). The context around the initiation ATG (nucleotides 2012-2026) was designed to match that found in highly expressed plant genes (Sawant et al., 1999). Nucleotides 2018-2086 comprise a sequence encoding a modified version of the signal peptide of Vicia faba legumin (Baumlein et al., 1986). Nucleotides 2087-2605 comprise a sequence encoding the von Willebrand factor type A domain of ATR (Bradley et al., 2001). Nucleotides 2606-3631 comprise a sequence encoding the human IgA2m(2) constant region (Chintalacharuvu et al., 1994). Nucleotides 3794-4108 derive from the agropine synthase (ags) terminator. Nucleotides 4530-4800 represent the NOS promoter (Depicker et al., 1982). Nucleotides 4835-5626 encode the npt II gene (conferring resistance to kanamycin). Nucleotides 5648-5870 are the polyadenylation signal from A. tumefactions gene 7 (Gielen et al., 1984). Nucleotides 6454-6602 represent the left T-DNA border.

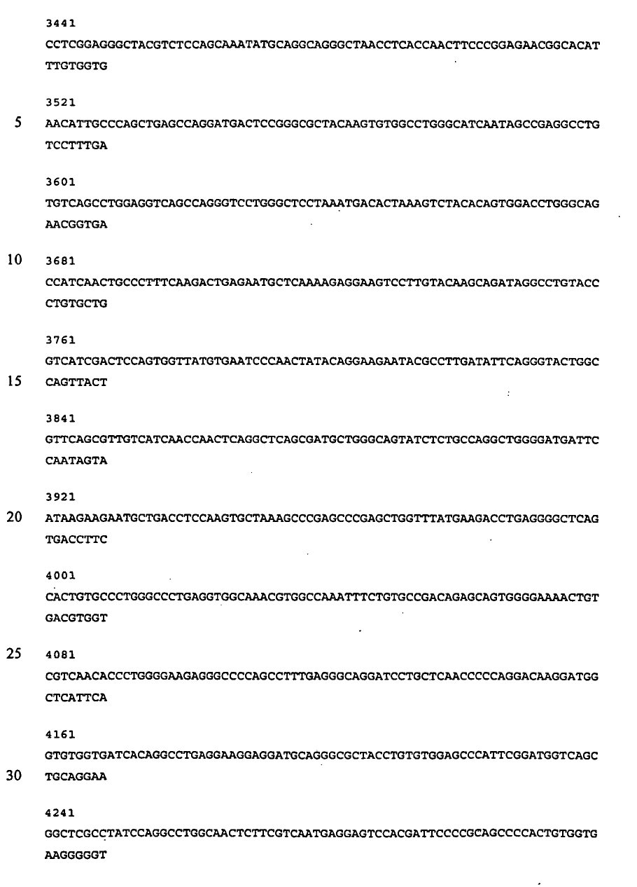

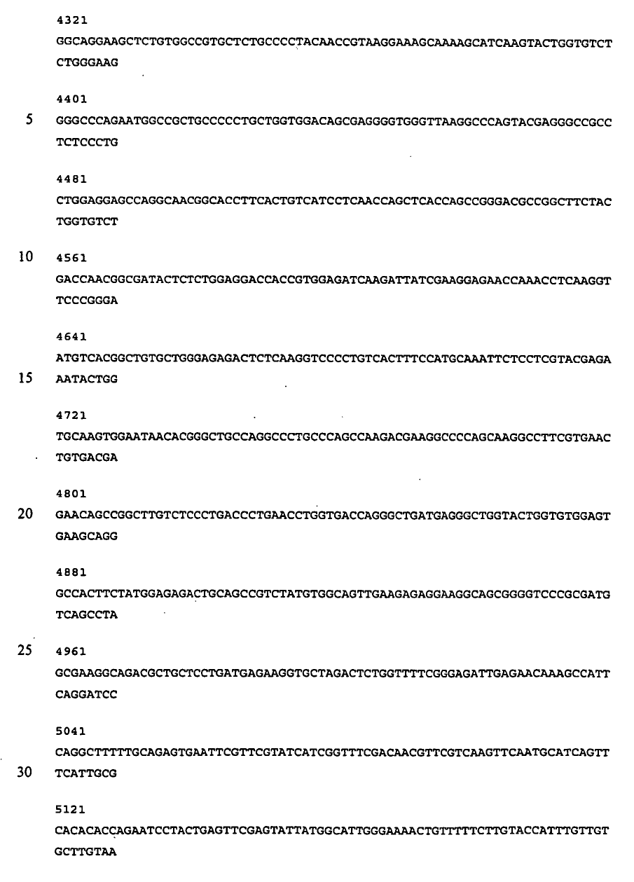

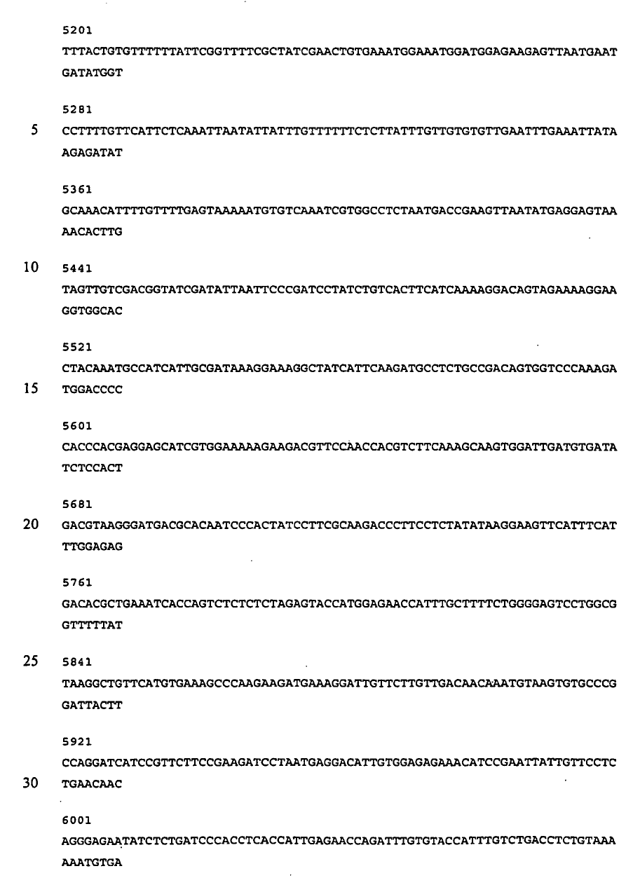

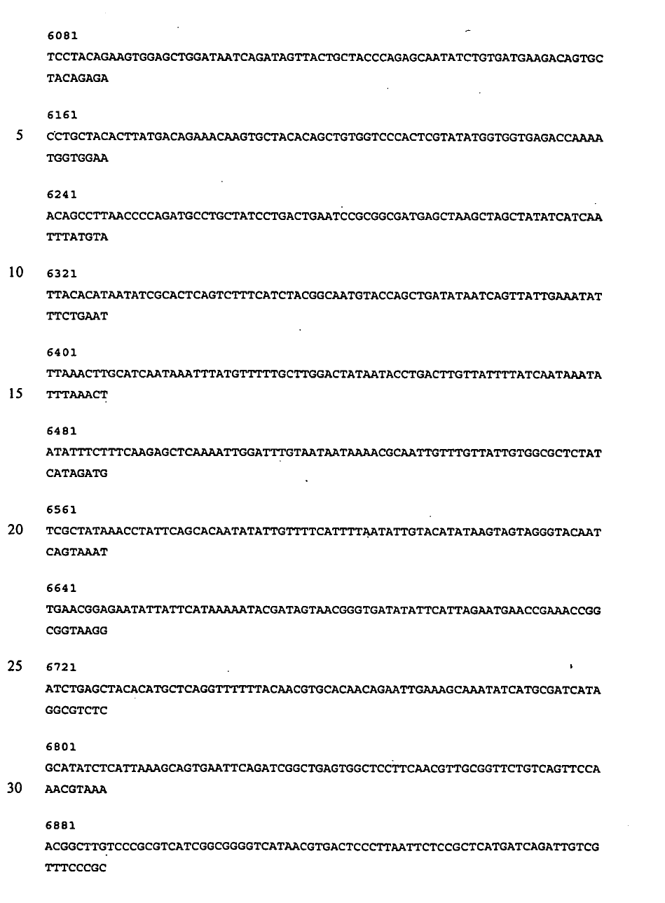

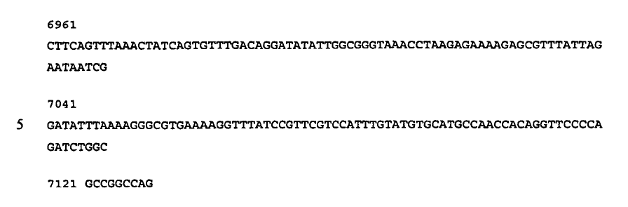

A construct for the expression in plants of human J chain and secretory component has also been developed. This construct, pGPTV-hpt-ocs-35SJ/SC, is based on the vector pGPTV-hpt-ocs, derived from pGPTV-hpt in the same manner as described for pGPTV-kan-ocs above. Sequence between the T-DNA borders of the plasmid pGPTV-hpt-ocs-35SJ/SC is shown in FIG. 12. Sequence outside the left and right borders are as described (Becker et al., 1992). Nucleotides 1-149 represent the left T-DNA border. Nucleotides 733-955 (complement) represent the polyadenylation signal from A. tumefactions gene 7 (Gielen et al., 1984). Nucleotides 980-2002 (complement) represent the hpt gene (conferring resistance to hygromycin). Nucleotides 2049-2318 (complement) represent the NOS promoter (Depicker et al., 1982). Nucleotides 2898-3230 represent the cauliflower mosaic virus (CaMV) 35S promoter driving expression of the human secretory component gene including it's native signal peptide (nucleotides 3236-5056), and nucleotides 5060-5445 represent the polyadenylation signal from the pea rbcS-E9 gene (Mogen et al., 1992). Nucleotides 5457-5788 represent a second copy of the CaMV 35S promoter driving expression of the human Joining (J) chain gene including it's native signal peptide (nucleotides 5797-6273), and nucleotides 6281-6494 represent the gene 7 terminator. Nucleotides 6501-6819 (complement) represent the ocs 3′ terminator region. Nucleotides 6944-7113 represent the right T-DNA border.

11. Plant Transformation and ATR Immunoadhesin Expression in Plants

The expression cassettes described above are used to produce the assembled immunoadhesin in plants, via Agrobacterium-mediated transformation. Plasmids pGPTV-hpt-ocs-35SJ/SC and pGPTV-kan-ocs-ATR-IgA2 are introduced separately into A tumefaciens strain LBA4404. Overnight cultures of both strains are used for simultaneous “co-cultivation” with leaf pieces of tobacco, according to a standard protocol (Horsch et al., 1985). Transformed plant tissue is selected on regeneration medium containing both kanamycin (100 μg/mL) and hygromycin (25 μg/mL).

Plantlets that regenerate in the presence of antibiotic are screened for transgene expression. This is accomplished by preparing extracts of leaf tissue in phosphate buffered saline (PBS) and spotting clarified extracts on nitrocellulose paper. These “dot” blots are probed with alkaline-phosphatase-conjugated antisera specific for human IgA, J chain or secretory component. Plants that test positive on this first screen are subjected for further screens involving western blotting and PCR. The ATR-IgA2 immunoadhesin is expected to have a subunit MW of 59 kDa. Due to natural dimerization of the heavy chain constant region, dimers of ˜118 kDa are also expected to form. These dimers further dimerize within the plant cell in the presence of J chain, forming a molecule of ˜252 kDa. With the addition of secretory component, a molecular species of ˜320 kDa is observed.

The presence of a signal peptide in the chimeric heavy chain, J chain and secretory component constructs is important for assembly into a multimeric immunoadhesin. Upon translation of the mRNAs, signal peptide cleavage is predicted to deposit the each protein into the plant cell's endoplasmic reticulum (ER). Assembly into a multimeric immunoadhesin is expected to take place in the ER and golgi bodies, and the assembled molecule is then secreted from the cell.

12. Purification of Assembled ATR Immunoadhesin

Purification of Assembled ATR Immunoadhesin can be accomplished essentially as described for the ICAM-1 mmunoadhesin of Example 3, supra.

13. The ATR Immunoadhesin Inhibits Toxin Action on Mammalian Cells

The expression cassettes described above are used to produce the assembled immunoadhesin, which is purified from plant extracts. The purified immunoadhesin is used to protect CHO-K1 cells from being killed in a simple bioassay. CHO-K1 cells have the receptor to which PA binds on their cell surfaces, but they are not sensitive to the toxin. They are killed when challenged with PA and LFN-DTA, a fusion protein composed of the N-terminal 255 amino acids of LF linked to the catalytic A chain of diptheria toxin. This recombinant toxin exploits the same LF-PA-receptor interactions that are required for the binding and entry of the native LF and OF proteins. To test the protective effect of the immunoadhesin, CHO-K1 cells are mixed with an increasing amount of ATR-IgA2 in the presence of a constant (toxic) amount of PA and LF_{N}-DTA, and the subsequent effect on protein synthesis is measured. ATR-IgA2 is an effective inhibitor of toxin action, inhibiting toxin action at a lower molar concentration than soluble ATR.

14. The ATR Immunoadhesin Inhibits Toxin Action in Human Subjects

The purified immunoadhesin is prepared in a pharmaceutically acceptable buffer and is administered to human subjects infected with Anthrax. The route of administration may be either as an inhaled aerosol or as an injection. Subjects in late stages of infection who would normally die are protected from toxin action by the immunoadhesin.

15. Construction of an Alternative ATR Immunoadhesin Expression Cassette

A cassette encoding the entire extracellular portion of human ATR (amino acids 24-320) is prepared by PCR cloning. Specifically, a fragment of 878 bp is amplified from plasmid ATR (Bradley et al., 2001), or from plasmid TEM8 (St Croix et al., 2000) using the following oligonucleotide primers:

| | |
|---|---|
| 5′- [symbol] GGGGGTC | (SEQ ID NO: 95) |
| CAG -3′ | |
| 5′-GAGCTCCCGTCAGAACAGTGTGTGG | (SEQ ID NO: 96) |
| TGGTG -3′ | |

The second primer (SEQ ID NO: 96) is designed to introduce a Sac I site at the 3′ end of the coding region of the ATR extracellular domain (solid underline). PCR is performed with Pfu polymerase (Stratagene) to reduce accumulation of errors. A second fragment of 121 bp, which includes a 5′ untranslated region and a plant signal peptide, is amplified from plasmid δ ATG-TOPO#4, using the following oligonucleotide primers:

| | |
|---|---|
| 5′-GGTACCACTTCTCTCAATCCAACTTTC-3′ | (SEQ ID NO: 93) |
| 5′- [symbol] AGCCAAACTAGTA | (SEQ ID NO: 97) |
| GAGGTGAACAAAAGC -3′ | |

The first primer (SEQ ID NO: 93) is designed to introduce a Kpn I site at the 5′ end of the PCR fragment (solid underline). The two PCR fragments have 20 nt of complementary sequence (dotted underlines). The two PCR fragments are mixed together, and a fragment of 981 bp is amplified using SEQ ID NO: 93 and SEQ ID NO: 96. The resulting PCR fragment is cloned into a plant expression cassette to form a genetic fusion with human IgA2 in the same manner as the partial ATR extracellular domain (Example 1).

An alternate construction using this same method would amplify amino acids 41-227.

The foregoing examples are not limiting and merely representative of various aspects and embodiments of the present invention. All documents cited are indicative of the levels of skill in the art to which the invention pertains. The disclosure of each document is incorporated by reference herein to the same extent as if each had been incorporated by reference in its entirety individually, although none of the documents is admitted to be prior art.

One skilled in the art will readily appreciate that the present invention is well adapted to carry out the objects and obtain the ends and advantages mentioned, as well as those inherent therein. The methods and compositions described illustrate preferred embodiments, are exemplary, and are not intended as limitations on the scope of the invention. Certain modifications and other uses will occur to those skilled in the art, and are encompassed within the spirit of the invention, as defined by the scope of the claims.

It will be readily apparent to one skilled in the art that varying substitutions and modifications may be made to the invention without departing from the scope and spirit of the invention. Thus, such additional embodiments are within the scope of the invention and the following claims.

The invention illustratively described herein suitably may be practiced in the absence of any element or elements, limitation or limitations which is not specifically disclosed herein. The terms and expressions which have been employed are used as terms of description and not of limitation, and there is no intention in the use of such terms and expressions of excluding any equivalents of the features shown and described, or portions thereof. It is recognized that various modifications are possible within the scope of the invention claimed. Thus, it should be understood that although the present invention has been specifically disclosed by preferred embodiments, optional features, modifications and variations of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group, and exclusions of individual members as appropriate.

Other embodiments are within the following claims.

We claim:

1. An immunoadhesin comprising a chimeric toxin receptor protein, said toxin receptor comprising:
a toxin receptor protein linked to at least a portion of an immunoglobulin heavy chain; and
J chain and secretory component associated with said chimeric toxin receptor protein.

2. The immunoadhesin of claim 1 wherein said toxin receptor protein is an Anthrax toxin receptor protein comprised of
the extracellular domain of Anthrax toxin receptor or any portion thereof.

3. The immunoadhesin of claim 1 wherein said immunoglobulin heavy chain is selected from the group consisting of
IgA, IgA1, IgA2, IgM, and chimeric immunoglobulin heavy chains.

4. The immunoadhesin of claim 2 comprising at least one additional chimeric Anthrax toxin receptor protein.

5. The immunoadhesin of claim 2 wherein said Anthrax toxin receptor protein is comprised of any portion of the extracellular domain of Anthrax toxin receptor protein; and said immunoglobulin heavy chain comprises at least a portion of an IgA2 heavy chain.

6. The immunoadhesin of claim 1 expressed in transgenic plants.

7. The immunoadhesin of claim 1 expressed in monocotyledonous plants.

8. The immunoadhesin of claim 1 expressed in dicotyledonous plants.

9. The immunoadhesin of claim 1 wherein all proteins are human.

10. The immunoadhesin of claim 1 expressed in heterologous cells derived from plants vertebrates, or invertebrates.

11. The immunoadhesin of claim 1 expressed in mammalian cells.

12. The immunoadhesin of claim 1 expressed in hairy root cultures.

13. The immunoadhesin of claim 1 expressed in plant cells in tissue culture.

14. An immunoadhesin comprising a chimeric bacterial or viral toxin receptor protein, said toxin receptor protein comprising: a toxin receptor protein linked to at least a portion of an immunoglobulin heavy chain, wherein said immunoadhesin has plant-specific glycosylation.

15. The immunoadhesin of claim 14 wherein said toxin receptor protein is an Anthrax toxin receptor protein.

16. The immunoadhesin of claim 15 wherein said immunoadhesin further comprises a J chain and secretory component associated with said chimeric Anthrax toxin receptor protein.

17. The immunoadhesin of claim 15 wherein said Anthrax toxin receptor protein is comprised of the extracellular domain of Anthrax toxin receptor or any portion thereof.

18. The immunoadhesin of claim 14 wherein said immunoglobulin heavy chain is selected from the group of IgA, IgA₁, IgA₂, IgG₁, IgG₂, IgG₃, IgG₄, IgD, IgE, IgM, and a chimeric immunoglobulin heavy chain.

19. The immunoadhesin of claim 14 comprising at least one additional chimeric toxin receptor protein.

20. The immunoadhesin of claim 14 wherein said toxin receptor protein is comprised of any portion of the extracellular domain of said toxin receptor protein; and said immunoglobulin heavy chain comprises at least a portion of an IgA₂ heavy chain.

21. The immunoadhesin of claim 14 wherein all proteins are human or associated with a human host during infection and/or pathagenesis.

22. The immunoadhesin of claim 14 expressed in heterologous cells derived from plants vertebrates, or invertebrates.

23. The immunoadhesin of claim 14 expressed in hairy root cultures.

24. The immunoadhesin of claim 14 expressed in plant cells in tissue culture.

25. The immunoadhesin of claim 14 expressed in transgenic plants.

26. The immunoadhesin of claim 14 expressed in monocotyledonous plants.

27. The immunoadhesin of claim 14 expressed in dicotyledonous plants.

28. A composition comprising an immunoadhesin and plant material, wherein said immunoadhesin comprises a chimeric toxin receptor protein, said chimeric toxin receptor protein linked to at least a portion of an immunoglobulin heavy chain.

29. The composition of claim 28 further comprising a J chain and secretory component with said chimeric toxin receptor protein.

30. The composition of claim 28 wherein said chimeric toxin receptor protein is comprised of any portion of the extracellular domain of said toxin receptor protein; and said immunoadhesin has plant-specific glycosylation.

31. The composition of claim 28 wherein said immunoglobulin heavy chain is selected from the group consisting of IgA, IgA₁, IgA₂, IgG₁, IgG₂, IgG₃, IgG₄, IgD, IgE, IgM, and a chimeric immunoglobulin heavy chain.

32. The composition of claim 28 comprising at least one additional chimeric toxin receptor protein.

33. The composition of claim 28 wherein said toxin receptor protein is comprised of any portion of the extracellular domain of said toxin receptor protein; and said immunoglobulin heavy chain comprises at least a portion of an IgA₂ heavy chain.

34. The composition of claim 28 wherein said toxin receptor protein is an Anthrax toxin receptor protein.

35. A method for reducing the binding of a viral or bacterial antigen to a host cell, said method comprising: contacting said antigen with an the immunoadhesin of claim 1, and wherein said immunoadhesin binds to said antigen and reduces the toxic activity thereof.

36. A method for reducing mortality and morbidity of a viral or bacterial pathogen, said method comprising: contacting an antigen of said viral or bacterial pathogen with an the immunoadhesin of claim 1, and wherein said immunoadhesin binds to said antigen and reduces toxic activity thereof.

37. A method for reducing mortality and morbidity due to a bacterial or viral toxin in a human subject, said method comprising: administering to said subject an effective amount of an the immunoadhesin of claim 1, and wherein said immunoadhesin binds to said toxin and reduces the toxic activity thereof.

38. The method claim 35 wherein said, toxin is an anthrax PA toxin.

39. A pharmaceutical composition comprising the immunoadhesin of claim 1, in a pharmaceutically acceptable buffer.

40. An expression vector comprising a gene encoding a chimeric toxin receptor protein operatively linked to a plant promoter, said chimeric toxin receptor protein linked to at least a portion of an immunoglobulin heavy chain.

41. The expression vector claim 40 wherein said toxin receptor protein is an anthrax toxin receptor protein.

42. The immunoadhesin of claim 1 wherein said chimeric receptor protein comprises ICAM-1.
